(19) United States
(12) Patent Application Publication
(10) Pub. No.: US 2021/0404961 A1
(43) Pub. Date: Dec. 30, 2021

(54) METHODS AND USES FOR DETERMINING OSTEOGENIC POTENTIAL OF IN VITRO DIFFERENTIATED CELLS

(71) Applicant: BONE THERAPEUTICS S.A., Gosselies (BE)

(72) Inventors: Sandra Pietri, Moha (BE); Delphine De Troy, Schaerbeek (BE); Caroline Trus, Cheratte (BE); Sylvain Normand, Bruxelles (BE); Laure Hertzog, Werentzhouse (FR)

(21) Appl. No.: 17/279,294

(22) PCT Filed: Sep. 25, 2019
(86) PCT No.: PCT/EP2019/075794

§ 371 Date: Mar. 24, 2021

(30) Foreign Application Priority Data
Sep. 25, 2018 (EP) 18196721.7

(51) Int. Cl. G01N 21/64; C12N 5/077

(52) U.S. Cl. CPC G01N 21/6428; G01N 2021/6439; C12N 5/0655; C12N 5/0654

(57) ABSTRACT

The application provides the use of CD73, CD105, CD44 and/or CD10 for determining osteogenic potential of in vitro differentiated cells. The application further provides a method for determining osteogenic potential of in vitro differentiated cells comprising measuring the quantity of the in vitro differentiated cells expressing CD73, CD105, CD10 and/or CD44, and/or measuring the quantity of CD73, CD105 and/or CD44 expressed by the in vitro differentiated cells. The invention also provides a method for selecting a subject for preparing in vitro differentiated cells of chondro-osteoblastic lineage comprising recovering MSC from a biological sample of a subject; obtaining in vitro differentiated cells from the MSC; determining the osteogenic potential of the in vitro differentiated cells by a method as disclosed herein; and selecting the subject for preparing in vitro differentiated cells of chondro-osteoblastic lineage if the in vitro differentiated cells have clinically useful osteogenic potential.

### CROSS REFERENCE TO RELATED APPLICATIONS

This application is a national phase entry under 35 U.S.C. § 371 of International Patent Application PCT/EP2019/075794, filed Sep. 25, 2019, designating the United States of America and published in English as International Patent Publication WO 2020/064793 on Apr. 2, 2020, which claims the benefit under Article 8 of the Patent Cooperation Treaty to European Patent Application Serial No. 18196721.7, filed Sep. 25, 2018, the entireties of which are hereby incorporated by reference.

### FIELD OF THE INVENTION

The invention relates to methods and uses for determining osteogenic potential of in vitro differentiated cells. More particularly, the invention relates to methods and uses for determining osteogenic potential of in vitro differentiated cells including measuring one or more cellular markers.

### BACKGROUND OF THE INVENTION

Transplantation of stem cells capable of undergoing osteogenic differentiation, of cells that are committed towards osteogenic differentiation or of cells with bone-forming ability is a promising avenue for the treatment of bone-related diseases, in particular when the treatment requires production of new bone tissues.

Mesenchymal stem cells (MSC) have been used previously to treat bone disorders (Gangji et al., 2005 Expert Opin Biol Ther 5: 437-42). However, although such relatively undifferentiated stem cells can be transplanted, they are not committed to an osteoblastic lineage and contribution to the formation of bone tissue may be primarily mediated by paracrine effects. Moreover, the quantity of MSC obtainable from subjects for the therapeutic use is frequently unsatisfactory.

Several methods for in vitro expanding MSCs and obtaining osteoprogenitors, osteoblasts or osteoblast phenotype cells from MSCs have been developed. Cells obtained by such methods may have varying degrees of osteogenic potential in vitro and in vivo. As a result, the amount of new bone tissue produced in vivo upon transplantation of such cells may not always be predictable and in some cases not optimal for clinical purposes.

There exists a need to determine prior to transplantation of in vitro cultured cells, such as in vitro differentiated MSC-derived cells, whether the cells have an osteogenic potential which is clinically useful.

### SUMMARY OF THE INVENTION

As corroborated by the experimental section, which illustrates certain representative embodiments of the invention, the inventors realized that the osteogenic potential of in vitro differentiated cells, such as mesenchymal stem cells (MSC)-derived cells, can be evaluated by determining a specific cell surface marker expression profile of said cells. More particularly, the inventors found that by measuring the quantity of the in vitro differentiated cells expressing any one or more of CD73, CD105, CD10 or CD44, preferably all of CD73, CD105, CD10 and CD44, and measuring the quantity of any one or more of CD73, CD105 or CD44, preferably all of CD73, CD105 and CD44, expressed by the cells, one can determine whether the cells have osteogenic potential, more particularly osteogenic potential that renders the cells useful in clinical settings. Furthermore, the present inventors also found that using the method for determining the osteogenic potential of MSC-derived cells as disclosed herein a subject could be selected which is particularly suitable as a donor of MSCs for preparing MSC-derived cells of chondro-osteoblastic lineage.

Hence, in an aspect, the invention provides the use of any one or more of CD73, CD105 or CD44 for determining osteogenic potential of in vitro differentiated cells.

Preferably, the invention provides the use of CD73, CD105, CD44 and CD10 for determining osteogenic potential of in vitro differentiated cells.

In a further aspect the invention provides a method for determining osteogenic potential of in vitro differentiated cells comprising measuring the quantity of the in vitro differentiated cells expressing any one or more of CD73, CD105, CD10 or CD44, and measuring the quantity of any one or more of CD73, CD105 or CD44 expressed by the in vitro differentiated cells.

Preferably, the invention provides a method for determining osteogenic potential of in vitro differentiated cells comprising measuring the quantity of the in vitro differentiated cells expressing CD73, CD105, CD10 and CD44, and/or measuring the quantity of any one or more of CD73, CD105 or CD44 expressed by the in vitro differentiated cells.

In a further aspect the invention provides a method for selecting a subject for preparing in vitro differentiated cells of chrondro-osteoblastic lineage, the method comprising:
recovering MSC from a biological sample of a subject;
obtaining in vitro differentiated cells from the MSC;
determining the osteogenic potential of the in vitro differentiated cells by a method as taught herein; and
selecting the subject for preparing in vitro differentiated cells of chondro-osteoblastic lineage if the in vitro differentiated cells have clinically useful osteogenic potential.

These and further aspects and preferred embodiments of the invention are described in the following sections and in the appended claims. The subject-matter of the appended claims is hereby specifically incorporated in this specification.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the bone neo-formation on a murine bone calvaria coronal section evidenced by murine and human calcium-binding fluorochromes, 2 weeks after administration of excipient alone (control condition), MSC-derived bone-forming cells A (generated with FGF-2 and TGFβ1) or MSC-derived bone-forming cells B (generated with FGF-2, TGFβ1 and heparin).

FIG. 2 illustrates the quantification of bone formation (%) performed on murine calvaria coronal sections, 2 weeks after administration of excipient alone (negative control), MSC-derived bone-forming cells A (generated with FGF-2 and TGFβ1) or MSC-derived bone-forming cells B (generated with FGF-2, TGFβ1 and heparin).

FIG. 3 illustrates anti-murine and anti-human type I collagen double immunostaining (immunofluorescence) performed on murine bone calvaria coronal sections 2 weeks after administration of MSC-derived bone-forming cells B (generated with FGF-2, TGFβ1 and heparin). FIG. 3A illustrates anti-human and anti-murine type I collagen double immunostaining (merge) while FIG. 3B and FIG. 3C show anti-human and anti-murine type I collagen immunostaining respectively.

FIG. 4 illustrates histology staining of murine bone calvaria coronal sections, 2 weeks after administration of excipient alone, MSC, MSC-derived bone-forming cells A generated with FGF-2 and TGFβ1 (b-f cells A), or MSC-derived bone-forming cells B generated with FGF-2, TGFβ1 and heparin (b-f cells B). FIG. 4A calcium-binding fluorochromes were sequentially injected intraperitoneally (alizarin-red→calcein green→calcein blue→tetracycline) to evidence the bone neo-formation (arrows) and evaluate the dynamic of the bone-formation; FIG. 4B immunofluorescence (IF) human+murine type I collagen; FIG. 4C IF murine type I collagen; FIG. 4D IF human type I collagen. Anti-human and anti-murine type I collagen double immunofluorescence was performed to allow the detection of human and murin type I collagen secreted by the bone matrix; FIG. 4E ALP+Goldner staining: ALP: detection of the osteoblast activity in black (full lines and areas), Masson's trichrome Goldner: detection of the osteoid (unmineralized bone tissue) in black dotted lines, mineralized bone in dark grey lines; FIG. 4F tartrate-resistant acid phosphatase (TRAP): detection of the osteoclast activity in dark grey/black.

FIG. 5 represents photographs illustrating the bone neo-formation on murine bone calvaria coronal sections 2 weeks after administration of excipient alone; MSC; MSC-derived bone-forming cells A generated with FGF-2, TGFβ1 (b-f cells A); or MSC-derived bone-forming cells B generated with FGF-2, TGFβ1 and heparin (b-f cells B). The bone neo-formation is evidenced by fluorescence (labeled by the sequential integration of different fluorochromes: alizarin-red→calcein green→calcein blue→tetracycline yellow). Red, green and blue staining appears in light grey and bone neo-formation thickness is indicated with double arrows. Yellow staining has been surrounded by dotted lines.

FIG. 6 represents a graph illustrating the total surface area of neo-formed bone (means±SEM, *p<0.05) measured on murine calvaria sections 2 weeks after administration of MSC (dark grey) or bone-forming cells B (light grey).

FIG. 7 illustrates safranin-orange staining of cartilaginous matrix (surrounded by dashes lines) of mineralized nodules performed on murine bone calvaria sagittal sections one day after the administration of bone-forming cells B (D1) and over time (D7, D14, D21) up to 28 days (D28) after administration.

FIG. 8 illustrates the effect of MSC-derived cells in a segmental femoral sub-critical size defect model. FIG. 8A represents a graph illustrating the measurement of the defect size on X-ray images on the day of the surgical procedure/item administration (D0) and over time (1, 2, 3, 4, 5 weeks) up to 6 weeks (6 W) after administration of the excipient alone, bone-forming cells A (b-f cells A) or bone-forming cells B (b-f cells B); means±SEM, **p<0.01, ***p<0.001; FIG. 8B represents representative X-ray images of segmental femoral defects at D0 and 6 W after administration of the excipient alone or bone-forming cells B (b-f cells B); FIG. 8C represents a graph illustrating the volume measurement of bone repair by micro-computed tomography (micro-CT) analyses at 6 W after administration of the excipient alone (n=7) and bone-forming cells B (n=8); mean±SEM, *p<0.05.

FIG. 9 illustrates the flow cytometry gating strategy used in Example 5.

FIG. 10 illustrates CD73 (upper panel) and CD44 (lower panel) expression levels analysed by flow cytometry in MSC, bone-forming cells A, B and C. (N=12, 6, 22, 15 (CD73) and N=22, 8, 22, 18 (CD44) for MSC, bone-forming cells A, B and C respectively, wherein N represents the number of individual experiments).

FIG. 11 illustrates the osteoinduction and osteogeny assessed by X-ray analysis. FIG. 11A: Osteoinduction (A, left panel) is assessed by measuring the grey intensity value which is directly correlated to the bone opacity and therefore to the bone thickness. Osteogeny (A, right panel) is assessed by measuring the surface of the nodule that appears more refringent by X-ray imaging. FIG. 11B: The bone opacity is significantly higher for bone-forming cells C cryopreserved (“B-F cells C”) compared to excipient (n=20 (excipient) and n=34 (B-F cells C from 5 different batches). FIG. 11C: The surface of osteogeny is significantly higher compared to excipient in which no mineralized nodules were observed (n=20 (excipient) and n=34 (B-F cells C from 5 different batches). FIG. 11D-FIG. 11E: Osteoinduction with (FIG. 8D) or without (FIG. 8E) osteogeny (represented by the absolute bone formation) is significantly higher for bone-forming cells C cryopreserved (“B-F cells C”) compared to excipient. Mann Whitney U-test: ***p≤0.001. FIG. 11F: in addition to osteoinduction activities, bone-forming cells C cryopreserved (“B-F cells C”) promote a high osteogenic activity indicated by the presence of at least one mineralized nodule in 4/5 bone marrow donors (or batch production) and 65% of mice (n=20 (excipient) and n=34 (B-F cells C from 5 different batches).

FIG. 12 illustrates coronal histological section 4 weeks after a single administration of bone-forming cells C cryopreserved or excipient. Bone-forming cells C cryopreserved display activity through two mechanisms: (i) “osteoinduction”: stimulation of host bone formation through paracrine secretion leading to intramembranous ossification and (ii) “osteogeny”: promotion of “direct” bone formation (from donor/human origin) by endochondral ossification.

FIG. 13 illustrates histology analysis of mice calvaria 4 weeks after having received a single injection of bone-forming cell C cryopreserved. Bone-forming cells C cryopreserved displayed osteoinduction and osteogenic properties (“fluo”). Human bone formation (“human type I collagen”) was highlighted in mineralized nodules (osteogeny). Osteoblast (“ALP” indicated by black arrows in the 3^{th} panel) and osteoclast (“TRAP”, indicated by black arrows in the 4^{th} panel) activities were mostly detected in mineralized nodules showing that the bone remodeling process in the nodules was still ongoing 4 weeks post-administration. No osteoid (“Goldner's Masson trichrome staining”) was highlighted indicating that the bone formation process is completed.

FIG. 14 illustrates the effect of bone-forming cells C cryopreserved (“B-F cells C”) in a segmental femoral sub-critical size defect model. X-ray images represent segmental femoral defects at Day 0 until Week 10 after administration of the excipient alone or bone-forming cells C cryopreserved.

FIG. 15 illustrates the effect of bone-forming cells C cryopreserved in a segmental femoral sub-critical size defect model (sub-CSD model). The graph represents the percentage of bone repair on X-ray images on the day of the surgical procedure/item administration (“W0”) and over time up to 10 weeks (“W10”) after administration of the excipient alone, or bone-forming cells C cryopreserved (“B-F cells C”); means±SEM, ***p<0.001 (two-way repeated measures ANOVA).

FIG. 16 illustrates the effect of bone-forming cells C cryopreserved in a segmental femoral sub-critical size defect model (sub-CSD model). The graph represents the RUS score determined from X-ray images on the day of the surgical procedure/item administration (“W0”) and over time up to 10 weeks (“W10”) after administration of the excipient alone, or bone-forming cells C cryopreserved (B-F cells C); means±SEM, **p<0.01, ***p≤0.001 (two-way repeated measures ANOVA).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations off 10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any 3 or more, 4 or more, 5 or more, 6 or more, or 7 or more etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The present inventors realised certain cell surface markers useful for determining osteogenic potential of in vitro differentiated cells. More particularly, the inventors found that the quantity of in vitro differentiated cells expressing any one or more of CD73, CD105, CD10 or CD44, preferably all of CD73, CD105, CD10 and CD44, and the quantity of any one or more of CD73, CD105 or CD44, preferably all of CD73, CD105 and CD44, expressed by the in vitro differentiated cells, can be used as markers for determining the osteogenic potential of the in vitro differentiated cells.

As used throughout the specification, references to ‘CD73’, ‘CD105’, ‘CD44’ or ‘CD10’ denote the respective peptides, polypeptides, proteins or nucleic acids, as apparent from the context, as commonly known under said designations in the art. The terms encompass such peptides, polypeptides, proteins or nucleic acids, of any organism where found, and particularly of animals, preferably warm-blooded animals, more preferably vertebrates, yet more preferably mammals, including humans and non-human mammals, still more preferably of humans.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “nucleic acid” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Exemplary modified nucleobases include without limitation 5-substituted pyrimidines, 6-azapyrimidines and N-2, N-6 and 0-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages. Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By means of additional guidance, CD73 is also known in the art as ecto-5′-nucleotidase, NT, eN, NT5, NTE, eNT, E5NT and CALJA. CD73 is a glycosyl-phosphatidylinositol (GPI)-linked cell surface enzyme. By means of an example, the human CD73 gene is annotated under NCBI Genbank (http://www.ncbi.nlm.nih.gov/) Gene ID 4907 and the human CD73 protein is annotated under Uniprot (www.uniprot.org) under accession number P21589.1. mRNA and protein sequences of human CD73 are annotated under the following NCBI Genbank accession numbers: CD73 mRNA (NM_002526.3; NM_001204813.1), CD73 protein (NP_002517.1; NP_001191742.1).

By means of additional guidance, CD105 is also know in the art as endoglin (ENG), END, FLJ41744, HHT1, ORW and ORW1. CD105 is a type I membrane glycoprotein located at the cell surface. By means of an example, human CD105 gene is annotated under NCBI Genbank Gene ID 2022 and human CD105 protein is annotated under Uniprot under accession number P17813.2. mRNA and protein sequences of human CD105 are annotated under the following NCBI Genbank accession numbers: CD105 (NM_001114753.2; NM_000118.3; NM_001278138.1), CD105 protein (NP_001108225.1; NP_000109.1; NP_001265067.1). By means of additional guidance, CD44 is also known in the art as homing cell adhesion molecule (HCAM), Pgp-1 (phagocytic glycoprotein-1), Hermes antigen, lymphocyte homing receptor (LHR), ECM-III, HUTCH-1, IN, MC56, MDU2, MDU3, MIC4, Pgp1, CDW44, CSPG8, HCELL, HUTCH-I and ECMR-III. CD44 is a cell-surface glycoprotein involved in cell-cell interactions, cell adhesion and migration. By means of an example, human CD44 gene is annotated under NCBI Genbank Gene ID 960 and human CD44 protein is annotated under Uniprot under accession number P16070.3. mRNA and protein sequences of human CD44 are annotated under the following NCBI Genbank accession numbers: CD44 mRNA (NM_000610.3, NM_001001389.1, NM_001001390.1, NM_001001391.1, NM_001001392.1, NM_001202555.1, NM_001202556.1, NM_001202557.1), CD44 protein (NP_000601.3, NP_001001389.1, NP_001001390.1, NP_001001391.1, NP_001189484.1, NP_001189484.1, NP_001189485.1, NP_001189486.1).

By means of additional guidance, CD10 is also known in the art as membrane metalloendopeptidase (MME), NEP, SFE, CALLA, CMT2T and SCA43. CD10 is a type II transmembrane glycoprotein. By means of an example, human CD10 gene is annotated under NCBI Genbank (http://www.ncbi.nlm.nih.gov/) Gene ID 4311 and human CD10 protein is annotated under Uniprot under accession number P08473.2. mRNA and protein sequences of human CD10 are annotated under the following NCBI Genbank accession numbers: CD10 mRNA (NM_000902.3, NM_007287.2, NM_007288.3, NM_007289.3, NM_001354642.1, NM_001354643.1, NM_001354644.1), CD10 protein (NP_000893.2, NP_009218.2, NP_009219.2, NP_009220.2, NP_001341571.1, NP_001341572.1, NP_001341573.1). The reader is reminded that where Genbank or Uniprot entries provide the sequence of precursor polypeptides or proteins, the corresponding mature forms (such as, for example, lacking signal peptides) would be expected to be present on the cell surface of cells.

The terms ‘CD73’, ‘CD105’, ‘CD44’ and ‘CD10’ particularly encompass such peptides, polypeptides, proteins, or nucleic acids, with a native sequence, i.e., ones of which the primary sequence is the same as that of the peptides, polypeptides, proteins, or nucleic acids found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of peptides, polypeptides, proteins, or nucleic acids are intended herein. Accordingly, all sequences of peptides, polypeptides, proteins, or nucleic acids found in or derived from nature are considered “native”. The terms encompass the peptides, polypeptides, proteins, or nucleic acids when forming a part of a living cell.

A first aspect provides the use of any one or more (e.g., one, two or all three) of CD73, CD105 or CD44 for determining osteogenic potential of in vitro differentiated cells. Hence, also provided is the use of one of CD73, CD105 or CD44 for determining osteogenic potential of in vitro differentiated cells; the use of two of CD73, CD105 or CD44 for determining osteogenic potential of in vitro differentiated cells; and the use of all three of CD73, CD105 and CD44 for determining osteogenic potential of in vitro differentiated cells.

Certain embodiments provide the use of any one or more (e.g., one, two, three or all four) of CD73, CD105, CD44 or CD10 for determining osteogenic potential of in vitro differentiated cells.

Certain embodiments provide the use of CD44 for determining osteogenic potential of in vitro differentiated cells. Certain embodiments provide the use of CD44 and any one or both of CD73 and CD105 for determining osteogenic potential of in vitro differentiated cells. Certain embodiments provide the use of CD44 and any one or more (e.g., one, two or all three) of CD73, CD105 or CD10 for determining osteogenic potential of in vitro differentiated cells.

Certain embodiments provide the use of CD10 for determining osteogenic potential of in vitro differentiated cells. Certain embodiments provide the use of CD10 and any one or more (e.g., one, two or all three) of CD73, CD105 or CD44 for determining osteogenic potential of in vitro differentiated cells.

The term “osteogenic potential” as used herein refers to the ability of cells to (trans)differentiate into bone-matrix-secreting cells or to the ability of cells to secrete bone matrix (i.e. without the need of a (trans)differentiation step), in vivo, and optionally in vitro. The term encompasses the ability of cells to form bone tissue by intramembranous ossification or endochondral ossification. The ability of the cells to form bone tissue by intramembranous ossification typically represents the ability of the cells to form bone tissue without the need of a calcified cartilage matrix as a template. The ability of the cells to form bone tissue by endochondral ossification typically represents the ability of the cells to form bone tissue by first forming a calcified cartilage matrix and subsequently using said calcified cartilage matrix as a template for bone tissue formation. The term does not encompass the osteoinductive potential of cells, which represents the ability of cells to attract other bone-matrix-secreting cells and/or to induce the (trans)differentiation of other cells into bone-matrix-secreting cells. The skilled person will understand that whereas the present aim is to determine the osteogenic potential of the in vitro differentiated cells, the cells may but need not, in addition to the osteogenic potential, also have osteoinductive potential.

In particular embodiments, the osteogenic potential is the potential of cells to form bone matrix by endochondral ossification.

The term “endochondral ossification” as used throughout the application refers to a process of bone tissue formation wherein first chondrocytes form a cartilage extracellular matrix, which is subsequently used by osteoblasts as a template for depositing bone matrix. During endochondral ossification, some of the chondrocytes can transdifferentiate into osteoblasts.

In preferred embodiments, all of CD73, CD105 and CD44 are used for determining osteogenic potential of in vitro differentiated cells.

In particular embodiments, CD10 is used in addition to any one or more of CD73, CD105 and CD44, preferably in addition to all of CD73, CD105 and CD44, for determining osteogenic potential of in vitro differentiated cells. Hence, also is provided the use of CD73, CD105, CD44 and CD10 for determining osteogenic potential of in vitro differentiated cells.

A further aspect provides a method for determining osteogenic potential of in vitro differentiated cells comprising (a1) measuring the quantity of the in vitro differentiated cells expressing any one or more (e.g., one, two, three or all four) of CD73, CD105, CD10 or CD44, and/or (a2) measuring the quantity of any one or more (e.g., one, two or all three) of CD73, CD105 or CD44 expressed by the in vitro differentiated cells. Hence, also provided herein is a method for determining osteogenic potential of in vitro differentiated cells comprising (a1) measuring the quantity of the in vitro differentiated cells expressing one of CD73, CD105, CD10 or CD44, preferably measuring the quantity of the in vitro differentiated cells expressing two of CD73, CD105, CD10 or CD44, more preferably measuring the quantity of the in vitro differentiated cells expressing three of CD73, CD105, CD10 or CD44, most preferably measuring the quantity of the in vitro differentiated cells expressing all four of CD73, CD105, CD10 and CD44; and/or (a2) measuring the quantity of one of CD73, CD105 or CD44 expressed by the in vitro differentiated cells, preferably measuring the quantity of two of CD73, CD105 or CD44 expressed by the in vitro differentiated cells, most preferably measuring the quantity of all three of CD73, CD105 or CD44 expressed by the in vitro differentiated cells. Hence, also provided herein is a method for determining osteogenic potential of in vitro differentiated cells comprising (a1) measuring the quantity of the in vitro differentiated cells expressing all four of CD73, CD105, CD10 and CD44, and/or (a2) measuring the quantity of all three of CD73, CD105 or CD44 expressed by the in vitro differentiated cells. In particular embodiments, the method as taught herein does not involve measuring other markers in addition to CD73, CD105, CD10 and CD44.

In certain embodiments, the methods as taught herein comprise measuring the quantity of the in vitro differentiated cells expressing any one or more (e.g., one, two, three or all four) of CD73, CD105, CD10 or CD44.

In certain embodiments, the methods as taught herein comprise measuring the quantity of the in vitro differentiated cells expressing CD44. In certain embodiments, the methods as taught herein comprise measuring the quantity of the in vitro differentiated cells expressing CD44 and any one or both of CD73 and CD105. In certain embodiments, the methods as taught herein comprise measuring the quantity of the in vitro differentiated cells expressing CD44 and any one or more (e.g., one, two or all three) of CD73, CD105 or CD10.

In certain embodiments, the methods as taught herein comprise measuring the quantity of the in vitro differentiated cells expressing CD10. In certain embodiments, the methods as taught herein comprise measuring the quantity of the in vitro differentiated cells expressing CD10 and any one or more (e.g., one, two or all three) of CD73, CD105 or CD44.

In certain embodiments, the methods as taught herein comprise measuring the quantity of any one or more (e.g., one, two or all three) of CD73, CD105 or CD44 expressed by the in vitro differentiated cells. In certain embodiments, the methods as taught herein comprise measuring the quantity of CD73, CD105 and CD44 expressed by the in vitro differentiated cells.

In certain embodiments, the methods as taught herein comprise measuring the quantity of CD73 expressed by the in vitro differentiated cells. In certain embodiments, the methods as taught herein comprise measuring the quantity of CD73 and any one or both of CD105 and CD44 expressed by the in vitro differentiated cells. In certain embodiments, the methods as taught herein comprise measuring the quantity of CD73 and any one or more (e.g., one, two or all three) of CD105, CD44 or CD10 expressed by the in vitro differentiated cells.

In certain embodiments, the methods as taught herein comprise measuring the quantity of CD105 expressed by the in vitro differentiated cells. In certain embodiments, the methods as taught herein comprise measuring the quantity of CD105 and any one or both of CD73 and CD44 expressed by the in vitro differentiated cells. In certain embodiments, the methods as taught herein comprise measuring the quantity of CD105 and any one or more (e.g., one, two or all three) of CD73, CD44 or CD10 expressed by the in vitro differentiated cells.

In certain embodiments, the methods as taught herein comprise measuring the quantity of CD44 expressed by the in vitro differentiated cells. In certain embodiments, the methods as taught herein comprise measuring the quantity of CD44 and any one or both of CD73 and CD105 expressed by the in vitro differentiated cells. In certain embodiments, the methods as taught herein comprise measuring the quantity of CD44 and any one or more (e.g., one, two or all three) of CD73, CD105 or CD10 expressed by the in vitro differentiated cells.

In certain embodiments, the methods as taught herein comprise measuring the quantity of any one or more (e.g., one, two, three or all four) of CD73, CD105, CD44 or CD10 on the cell surface of or expressed by the in vitro differentiated cells. In certain embodiments, the methods as taught herein comprise measuring the quantity of CD73, CD105, CD44 and CD10 on the cell surface of or expressed by the in vitro differentiated cells.

In certain embodiments, the methods as taught herein comprise measuring the quantity of CD10 expressed by the in vitro differentiated cells. In certain embodiments, the methods as taught herein comprise measuring the quantity of CD10 and any one or more (e.g., one, two or all three) of CD73, CD105 or CD44 expressed by the in vitro differentiated cells.

In certain embodiments, the methods as taught herein comprise (a1) measuring the quantity of the in vitro differentiated cells expressing CD73, CD105, CD10 and CD44, and/or (a2) measuring the quantity of any one or more (e.g., one, two or all three) of CD73, CD105 or CD44 expressed by the in vitro differentiated cells. In certain embodiments, the methods as taught herein comprise (a1) measuring the quantity of the in vitro differentiated cells expressing CD73, CD105, CD10 and CD44, and/or (a2) measuring the quantity of any one or more (e.g., one, two, three or all four) of CD73, CD105, CD44 or CD10 expressed by the in vitro differentiated cells.

The term “expressing” or “expression” as used throughout this specification generally encompasses the generation of any transcription or translation product, such as RNA, peptides, polypeptides and proteins, by a cell, as well the presentation of peptides, polypeptides or proteins on the cell surface.

By means of additional guidance, when a cell is said to be positive for or to express or comprise expression of a given gene, peptide, polypeptide or protein, such as CD73, CD105, CD10 or CD44, a skilled person would conclude the presence or evidence of a distinct signal for the gene, peptide, polypeptide or protein when carrying out a measurement capable of detecting or quantifying the gene, peptide, polypeptide or protein in or on the cell. Suitably, the presence or evidence of the distinct signal for the gene, peptide, polypeptide or protein would be concluded based on a comparison of the measurement result obtained for the cell to a result of the same measurement carried out for a negative control (for example, a cell known to not express the marker) and/or a positive control (for example, a cell known to express the marker).

A molecule or analyte such as a peptide, polypeptide, protein, or nucleic acid, or a group of two or more molecules or analytes such as two or more peptides, polypeptides, proteins, or nucleic acids, is “measured” in a sample when the presence or absence and/or quantity of said molecule or analyte or of said group of molecules or analytes is detected or determined in the sample, preferably substantially to the exclusion of other molecules and analytes. The terms “quantity”, “amount” and “level” are synonymous and generally well-understood in the art. The terms as used herein may particularly refer to an absolute quantification of a number of cells, a peptide, polypeptide, protein, or nucleic acid in a sample, or to a relative quantification of a number of cells, a peptide, polypeptide, protein, or nucleic acid in a sample, i.e., relative to another value such as relative to a reference value as taught herein. The quantity of a peptide, polypeptide or protein may also be represented by the activity of a peptide, polypeptide or protein. Activity of peptide, polypeptide or protein in a sample may also be expressed in absolute terms, e.g., in enzymatic units per volume, or relative terms.

An absolute quantity of a peptide, polypeptide, protein, or nucleic acid in a sample may be advantageously expressed as weight or as molar amount, or more commonly as a concentration, e.g., weight per volume or mol per volume.

A relative quantity of a peptide, polypeptide, protein, or nucleic acid in a sample may be advantageously expressed as an increase or decrease or as a fold-increase or fold-decrease relative to said another value, such as relative to a reference value as described elsewhere herein. Performing a relative comparison between first and second parameters (e.g., first and second quantities) may but need not require determining first the absolute values of said first and second parameters. For example, a measurement method may produce quantifiable readouts (such as, e.g., signal intensities) for said first and second parameters, wherein said readouts are a function of the value of said parameters, and wherein said readouts may be directly compared to produce a relative value for the first parameter vs. the second parameter, without the actual need to first convert the readouts to absolute values of the respective parameters.

A relative quantity of cells may be expressed as a percentage (fraction) of cells of the total number of cells analysed, more particularly of the total number of cells of which the expression of any one or more of CD73, CD105, CD10 or CD44 is determined. Accordingly, measuring the quantity of the in vitro differentiated cells expressing any one or more (e.g., one, two, three or all four) of CD73, CD105, CD10 or CD44 as taught herein may typically involve (i) determining the expression (i.e. presence) of CD73, CD105, CD10 and/or CD44 by in vitro differentiated cells, (ii) counting the number of in vitro differentiated cells determined to express CD73, CD105, CD10 and/or CD44 in step (i); and (iii) calculating the fraction of the in vitro differentiated cells determined to express CD73, CD105, CD10 and/or CD44 in step (i) relative to the total number of in vitro differentiated cells examined in step (i). The quantity of the in vitro differentiated cells expressing any one or more of CD73, CD105, CD10 or CD44 may be measured by any means known in the art. For example, by flow cytometry.

Accordingly, in particular embodiments, the quantity of the in vitro differentiated cells expressing any one or more (e.g., one, two, three or all four) of CD73, CD105, CD10 or CD44 is the fraction of the in vitro differentiated cells determined to express any one or more of CD73, CD105, CD10 or CD44 relative to all analysed in vitro differentiated cells.

Determining the presence and/or measuring the quantity of any one or more of CD73, CD105, CD10 or CD44 by in vitro differentiated cells may be performed by any existing, available or conventional detection and/or quantification methods used to measure the presence or absence (e.g., readout being present vs. absent; or detectable amount vs. undetectable amount) and/or quantity (e.g., readout being an absolute or relative quantity) of a peptide, polypeptide, protein, or nucleic acid in or on a cell or cell population. For example, such methods may include biochemical assay methods, immunoassay methods, mass spectrometry analysis methods, or chromatography methods, or combinations thereof.

In particular embodiments, measuring the presence and/or quantity of any one or more of CD73, CD105, CD10 or CD44 comprises measuring CD73, CD105, CD10 or CD44 peptides, polypeptides or proteins or CD73, CD105, CD10 or CD44 mRNA or both.

In preferred embodiments, measuring the presence and/or quantity of any one or more of CD73, CD105, CD10 or CD44 comprises measuring CD73, CD105, CD10 or CD44 peptides, polypeptides or proteins.

As each of the CD73, CD105, CD10 and CD44 peptides, polypeptides or proteins are typically expressed on the surface of cell, the skilled person will understand that if reference is made to any one or more of CD73, CD105, CD10 or CD44 on the cell surface, the peptide, polypeptide or protein is form is intended or that is reference is made to any one or more CD73, CD105, CD10 or CD44 peptides, polypeptides or proteins, any one or more of CD73, CD105, CD10 or CD44 on the cell surface is intended.

In particular embodiments, measuring the presence and/or the quantity of any one or more of CD73, CD105, CD10 or CD44 comprises measuring CD73, CD105, CD10 or CD44 peptides, polypeptides or proteins on the cell surface.

In more particular embodiments, the presence and/or quantity of any one or more of CD73, CD105, CD10 or CD44 peptides, polypeptides or proteins in a non-denaturated form on the cell surface of live cells are measured.

In more particular embodiments, the measuring of the presence and/or quantity of any one or more of CD73, CD105, CD10 or CD44 peptides, polypeptides or proteins comprises using a technique which employs one or more agents capable of specifically binding to CD73, CD105, CD10 or CD44, respectively, preferably wherein the one or more agents are, each independently, one or more antibodies, antibody fragments, antibody-like protein scaffolds, or aptamers.

Such methods may include affinity-based assay methods, wherein the ability of an assay to detect and/or quantify a peptide, polypeptide, protein, or nucleic acid is conferred by specific binding between a detectable and/or quantifiable binding agent and i) the peptide, polypeptide, protein, or nucleic acid. The binding agent may be an immunological binding agent (antibody) or a non-immunological binding agent. Examples of antibodies capable of binding to human CD73 include without limitation those available from the following vendors (“#” stands for catalogue number): BD Biosciences (allophycocyanin (APC)-conjugated mouse monoclonal antibody, #560847; Fluorescein isothiocyanate (FITC)-conjugated mouse monoclonal antibody, #561254; R-phycoerythrin (PE)-conjugated mouse monoclonal antibody #55027), Abcam (mouse monoclonal antibody, #ab54217; rabbit monoclonal, #ab79423; mouse monoclonal #ab34199), R&D systems (biotinylated polyclonal goat antibody, #BAF1182; monoclonal mouse, #MAB1182; PE-conjugated polyclonal goat antibody, #FAB8160P). Examples of antibodies capable of binding to human CD105 include without limitation those available from the following vendors (“#” stands for catalogue number): BD Biosciences (APC-conjugated mouse monoclonal antibody, #562408; PE-labelled mouse monoclonal antibody, #560839), Abcam (mouse monoclonal, #ab156756; mouse monoclonal, #ab2529; rabbit monoclonal, #ab221675), R&D systems (Alexa Fluor® 488-conjugated monoclonal mouse, #FAB10971G; Alexa Fluor® 647-conjugated monoclonal mouse, #FAB10971R; goat polyclonal, #AF1097). Examples of antibodies capable of binding to human CD44 include without limitation those available from the following vendors (“#” stands for catalogue number): BD Biosciences (PE-conjugated mouse monoclonal antibody, #550989; FITC-conjugated mouse monoclonal antibody, #555478), Abcam (rabbit polyclonal, #ab157107, PE-conjugated mouse monoclonal, #ab46793; PE-conjugated mouse monoclonal, #ab58754), R&D systems (Alexa Fluor®-conjugated rat monoclonal, #FAB6127 S; PE-conjugated mouse monoclonal, #FAB3660P). Examples of antibodies capable of binding to human CD10 include without limitation those available from the following vendors (“#” stands for catalogue number):BD Biosciences (PE-conjugated mouse monoclonal antibody, #555375), Abcam (rabbit monoclonal, #ab79423; rabbit polyclonal, #ab82073; PE-conjugated mouse monoclonal, #ab210380), R&D systems (Alexa Fluor®-conjugated mouse monoclonal, #FAB1182N; biotinylated goat polyclonal, #BAF1182). Affinity-based assay methods, such immunological assay methods, include without limitation immunohistochemistry, immunocytochemistry, flow cytometry, mass cytometry, fluorescence activated cell sorting (FACS), fluorescence microscopy, fluorescence based cell sorting using microfluidic systems, (immuno)affinity adsorption based techniques such as affinity chromatography, magnetic activated cell sorting or bead based cell sorting using microfluidic systems, immunoprecipitation, enzyme-linked immunosorbent assay (ELISA) and ELISPOT based techniques, radioimmunoassay (RIA), Western blot, etc.

Further techniques for detecting and/or quantifying peptides, polypeptides, or proteins, may be used, optionally in conjunction with any of the above described analysis methods. Such methods include, without limitation, mass spectrometric (MS) techniques, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The presence or absence and/or quantity of a nucleic acid, such as at the RNA level (e.g., at the level of hnRNA, pre-mRNA, mRNA, or cDNA), may be detected using standard quantitative RNA or cDNA measurement tools known in the art. Non-limiting examples include hybridisation-based analysis, microarray expression analysis, digital gene expression (DGE), RNA-in-situ hybridisation (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, or the like.

In further examples, any combinations of methods such as discussed herein may be employed.

In particular embodiments, CD73, CD105, CD10 or CD44 denote peptides, polypeptides, or proteins and the expression of any one or more of CD73, CD105, CD10 or CD44 denotes the cell-surface expression of any one or more of CD73, CD105, CD10 or CD44, respectively. The cell-surface expression of a peptide, polypeptide, or protein is preferably determined by flow cytometry.

In particular embodiments, the method as taught herein comprises (a1) measuring the fraction of the in vitro differentiated cells expressing any one or more of CD73, CD105, CD10 or CD44 on the cell surface of the in vitro differentiated cells; and/or (a2) measuring the quantity of any one or more of CD73, CD105 or CD44 on the cell surface of the in vitro differentiated cells. Hence, also provided herein is the method as taught herein comprising (a1) measuring the fraction of the in vitro differentiated cells expressing one of CD73, CD105, CD10 or CD44 on the cell surface of the in vitro differentiated cells, preferably measuring the fraction of the in vitro differentiated cells expressing two of CD73, CD105, CD10 or CD44 on the cell surface of the in vitro differentiated cells, more preferably measuring the fraction of the in vitro differentiated cells expressing three of CD73, CD105, CD10 or CD44 on the cell surface of the in vitro differentiated cells, most preferably measuring the fraction of the in vitro differentiated cells expressing all of CD73, CD105, CD10 and CD44 on the cell surface of the in vitro differentiated cells; and/or (a2) measuring the quantity of one of CD73, CD105 or CD44 on the cell surface of the in vitro differentiated cells, preferably measuring the quantity of two of CD73, CD105 or CD44 on the cell surface of the in vitro differentiated cells, more preferably measuring the quantity of all of CD73, CD105 and CD44 on the cell surface of the in vitro differentiated cells.

In certain embodiments, the methods as taught herein may comprise:
(a1) measuring the fraction of the in vitro differentiated cells expressing CD73, CD105, CD10 and CD44 on the cell surface of the in vitro differentiated cells;
(a2) measuring the quantity of any one or more (e.g., one, two or all three) of CD73, CD105 or CD44 on the cell surface of the in vitro differentiated cells;
(b1) comparing the fraction of the in vitro differentiated cells expressing CD73, CD105, CD10 and CD44 as measured in (a1) with a cut-off value representing cells having a known osteogenic potential;
(b2) comparing the quantity of any one or more (e.g., one, two or all three) of CD73, CD105 or CD44 as measured in (a2) with one or more respective cut-off values representing cells having a known osteogenic potential;
(c1) finding a deviation or no deviation of the fraction of the in vitro differentiated cells expressing CD73, CD105, CD10 and CD44 as measured in (a1) from said cut-off value;
(c2) finding a deviation or no deviation of the quantity of any one (e.g., one, two or all three) of CD73, CD105 or CD44 as measured in (a2) from said cut-off value; and
(d) attributing said deviation or no deviation to a particular determination of osteogenic potential of the in vitro differentiated cells.

In certain embodiments, the methods as taught herein may comprise:
(a1) measuring the fraction of the in vitro differentiated cells expressing CD73, CD105, CD10 and CD44 on the cell surface of the in vitro differentiated cells;
(a2) measuring the quantity of any one or more (e.g., one, two, three or all four) of CD73, CD105, CD44 or CD10 on the cell surface of the in vitro differentiated cells;
(b1) comparing the fraction of the in vitro differentiated cells expressing CD73, CD105, CD10 and CD44 as measured in (a1) with a cut-off value representing cells having a known osteogenic potential;
(b2) comparing the quantity of any one or more (e.g., one, two, three or all four) of CD73, CD105, CD44 or CD10 as measured in (a2) with one or more respective cut-off values representing cells having a known osteogenic potential;
(c1) finding a deviation or no deviation of the fraction of the in vitro differentiated cells expressing CD73, CD105, CD10 and CD44 as measured in (a1) from said cut-off value;
(c2) finding a deviation or no deviation of the quantity of any one (e.g., one, two, three or all four) of CD73, CD105, CD44 or CD10 as measured in (a2) from said cut-off value; and
(d) attributing said deviation or no deviation to a particular determination of osteogenic potential of the in vitro differentiated cells.

In particular embodiments, the measuring of the fraction of the in vitro differentiated cells expressing any one or more of CD73, CD105, CD10 or CD44 on the cell surface of the in vitro differentiated cells; and/or the measuring the quantity of any one or more of CD73, CD105 or CD44 on the cell surface of the in vitro differentiated cells is performed using a technique selected from the group consisting of flow cytometry, mass cytometry, fluorescence activated cell sorting, fluorescence microscopy, affinity separation, magnetic cell separation, microfluidic separation, and combinations thereof.

Flow cytometry encompasses methods by which individual cells of a cell population are analyzed by their optical properties (e.g., light absorbance, light scattering and fluorescence properties, etc.) as they pass in a narrow stream in single file through a laser beam. Flow cytometry methods include fluorescence activated cell sorting (FACS) methods by which a population of cells having particular optical properties are separated from other cells.

Elemental mass spectrometry-based flow cytometry, or mass cytometry, offers an approach to analyze cells by replacing fluorochrome-labelled binding reagents with mass tagged binding reagents, i.e., tagged with an element or isotope having a defined mass. In these methods, labeled particles are introduced into a mass cytometer, where they are individually atomized and ionized. The individual particles are then subjected to elemental analysis, which identifies and measures the abundance of the mass tags used. The identities and the amounts of the isotopic elements associated with each particle are then stored and analyzed. Due to the resolution of elemental analysis and the number of elemental isotopes that can be used, it is possible to simultaneously measure up to 100 or more parameters on a single particle.

Fluorescence microscopy broadly encompasses methods by which individual cells of a cell population are microscopically analyzed by their fluorescence properties. Fluorescence microscopy approaches may be manual or preferably semi-automated or automated.

Affinity separation also referred to as affinity chromatography broadly encompasses techniques involving specific interactions of cells present in a mobile phase, such as a suitable liquid phase (e.g., cell population in an aqueous suspension) with, and thereby adsorption of the cells to, a stationary phase, such as a suitable solid phase; followed by separation of the stationary phase from the remainder of the mobile phase; and recovery (e.g., elution) of the adsorbed cells from the stationary phase. Affinity separation may be columnar, or alternatively, may entail batch treatment, wherein the stationary phase is collected/separated from the liquid phases by suitable techniques, such as centrifugation or application of magnetic field (e.g., where the stationary phase comprises magnetic substrate, such as magnetic particles or beads). Accordingly, magnetic cell separation is also envisaged herein.

Microfluidic systems allow for accurate and high throughput cell detection, quantification and/or sorting, exploiting a variety of physical principles. Cell sorting on microchips provides numerous advantages by reducing the size of necessary equipment, eliminating potentially biohazardous aerosols, and simplifying the complex protocols commonly associated with cell sorting. The term “microfluidic system” as used throughout this specification broadly refers to systems having one or more fluid microchannels. Microchannels denote fluid channels having cross-sectional dimensions the largest of which are typically less than 1 mm, preferably less than 500 μm, more preferably less than 400 μm, more preferably less than 300 μm, more preferably less than 200 μm, e.g., 100 μm or smaller. Such microfluidic systems can be used for manipulating fluid and/or objects such as droplets, bubbles, capsules, particles, cells and the like. Microfluidic systems may allow for example for fluorescent label-based (e.g., employing fluorophore-conjugated binding agent(s), such as fluorophore-conjugated antibody(ies)), bead-based (e.g., bead-conjugated binding agent(s), such as bead-conjugated antibody(ies)), or label-free cell sorting (reviewed in Shields et al., Lab Chip. 2015, vol. 15: 1230-1249). In particular embodiments, the measuring of the fraction of the in vitro differentiated cells expressing any one or more of CD73, CD105, CD10 or CD44 on the cell surface of the in vitro differentiated cells; and the measuring the quantity of any one or more of CD73, CD105 or CD44 on the cell surface of the in vitro differentiated cells is performed using flow cytometry.

The in vitro differentiated cells may be labelled using antibodies conjugated to a fluorochrome (e.g., PE, PE-Cy 7, PE-Cy5, APC, APC-Cy7, Alexa Fluor 647®, Alexa Fluor 700®, FITC, Pacific Blue, Alexa Fluor 488®) for any one or more of CD73, CD105, CD10 or CD44 and subsequently excited by a laser at a certain wavelength (excitation wavelength of the fluorochrome) to emit light at varying wavelengths (emission wavelength of the fluorochrome). For example, in vitro differentiated cells may be labelled using an allophycocyanin (APC)-conjugated antibody against CD105 (BD Biosciences®, Cat No: 562408), an APC-conjugated antibody against CD73 (BD Biosciences®, Cat No: 560847), Phycoerythrin (PE)-conjugated antibody against CD10 (BD Biosciences®, Cat No: 555375) and/or a PE-conjugated antibody against CD44 (BD Biosciences®, Cat No: 550989). The skilled person will understand that the wavelength of the laser and excitation wavelength of the fluorochrome used to label the antibody has to be compatible. For example, the excitation wavelength for FITC is 488 nm and the emission wavelength is 500-560 nm; the excitation wavelength for PE is 488-561 nm and the emission wavelength is 560-595 nm. The presence or quantity of more than one of CD73, CD105, CD10 or CD44 can be measured simultaneously by using antibodies which are each conjugated to a different fluorochrome that emits a different light at a different wavelength.

Most cells do not naturally emit fluorescent light. Accordingly, if a fluorochrome-conjugated antibody is bound to any one or more of CD73, CD105, CD10 or CD44 on the cell surface of the in vitro differentiated cells, a fluorescent light signal will be picked up when that cell passes through the laser beam of the flow cytometer. For each of the fluorochrome-conjugated antibodies used in the flow cytometry analysis a positivity cut-off can be set. For example, a positivity cut-off may be set at 1% of positivity of the control isotype antibody.

The quantity of any one or more of CD73, CD105, CD10 or CD44 on the cell surface of the in vitro differentiated cells may be represented as the mean or median intensity of the fluorescent signal emitted by the in vitro differentiated cells. The mean or median intensity of the fluorescent signal is determined from the fluorescent signal detected for each cell of the whole analysed cell population (i.e. including the cells that did not emit a fluorescent light signal). More particularly, the quantity of any one or more of CD73, CD105, CD10 or CD44 on the cell surface of the in vitro differentiated cells may be represented by the normalized median fluorescence intensity (nMFI). The normalised nMFI is typically determined by dividing the MFI of the whole analysed cell population labeled with one or more fluorochrome-conjugated antibodies by the MFI of the negative control (e.g., cells labeled with one or more fluorochrome-conjugated isotype control antibodies, such as immunoglobulin G (IgG) control conjugated with FITC, APC and PE).

The “normalized Median of Fluorescence Intensity” or “nMFI” as used herein refers to the ratio of the MFI of the whole analyzed cell population labeled with one or more fluorochrome-conjugated antibodies (MFI_{marker_channel}) to the MFI of the cell population labeled with one or more fluorochrome-conjugated isotype control antibodies (MFI_{isotype_channel}), such as immunoglobulin G (IgG) control conjugated with a fluorochrome such as FITC, APC or PE. nMFI results are proportional to the quantity of markers present on cell surface of a population of interest. The (n)MFI is typically linked to the wavelength at which the emission of the fluorescent signal is measured. For example, excitation wavelengths may be 488 nm for FITC, 488 nm for PE and 633 nm for APC. For example, emission wavelengths may be 530 nm for FITC, 580 nm for PE and 660 nm for APC.

The flow cytometer may count the total number of labelled in vitro differentiated cells that pass through the laser as well as the number of labelled in vitro differentiated cells which emit light at a certain wavelength. This information can be used for determining the quantity, preferably the fraction, of in vitro differentiated cells expressing any one or more of CD73, CD105, CD10 or CD44, also known as the percentage of positive fluorescent cells (PPFC).

The skilled person will understand that before measuring the presence or quantity of any one or more of CD73, CD105, CD10 or CD44 on the cell surface of the in vitro differentiated cells the population of cells of interest may be distinguished from other cells or debris based on their forward and side scatter properties, for example by gating strategies.

Flow cytometry data analysis may be performed on a fixed number of detected events (e.g., a number of cells passing through the laser of the flow cytometer). For example, flow cytometry data analysis may be performed on 10 000 events of the gated cell population.

Flow cytometry may be performed using any cytometer known in the art. For example, using FACS CantoII (BD Biosciences®).

Flow cytometry data analysis may be performed using any flow cytometry software known in the art. For example, FACS Diva® 8.0 software (BD Biosciences®). In particular embodiments, the method as taught herein comprises (b1) comparing the quantity of the in vitro differentiated cells expressing any one or more (e.g., one, two, three or all four) of CD73, CD105, CD10 or CD44 as measured as described elsewhere herein with a reference value or cut-off value representing cells having a known osteogenic potential and (b2) comparing the quantity of any one or more (e.g., one, two or all three) of CD73, CD105 or CD44 expressed by the in vitro differentiated cells as measured as described elsewhere herein with a reference value or cut-off value representing cells having a known osteogenic potential. Hence, also provided herein is the method as taught herein comprising (b1) comparing the quantity of the in vitro differentiated cells expressing all of CD73, CD105, CD10 and CD44 as measured as described elsewhere herein with a reference value or cut-off value representing cells having a known osteogenic potential and (b2) comparing the quantity of all of CD73, CD105 and CD44 expressed by the in vitro differentiated cells as measured as described elsewhere herein with a reference value or cut-off value representing cells having a known osteogenic potential.

In more particular embodiments, the method as taught herein comprises (b1) comparing the fraction of the in vitro differentiated cells expressing any one or more (e.g., one, two, three or all four) of CD73, CD105, CD10 or CD44 as measured as described elsewhere herein with a cut-off value representing cells having a known osteogenic potential and/or (b2) comparing the quantity of any one or more (e.g., one, two or all three) of CD73, CD105 or CD44 expressed by the in vitro differentiated cells as measured as described elsewhere herein with a cut-off value representing cells having a known osteogenic potential. Hence, also provided herein is the method as taught herein comprising (b1) comparing the fraction of the in vitro differentiated cells expressing all of CD73, CD105, CD10 and CD44 as measured as described elsewhere herein with a cut-off value representing cells having a known osteogenic potential; and/or (b2) comparing the quantity of all of CD73, CD105 and CD44 expressed by the in vitro differentiated cells as measured as described elsewhere herein with a cut-off value representing cells having a known osteogenic potential.

In more particular embodiments, the method as taught herein comprises (b1) comparing the fraction of the in vitro differentiated cells expressing CD73, CD105, CD10 and CD44 as measured as described elsewhere herein with a cut-off value representing cells having a known osteogenic potential; and/or (b2) comparing the quantity of any one or more (e.g., one, two or all three) of CD73, CD105 or CD44 expressed by the in vitro differentiated cells as measured as described elsewhere herein with a cut-off value representing cells having a known osteogenic potential. In certain embodiments, the method as taught herein comprises (b1) comparing the fraction of the in vitro differentiated cells expressing CD73, CD105, CD10 and CD44 as measured as described elsewhere herein with a cut-off value representing cells having a known osteogenic potential; and/or (b2) comparing the quantity of any one or more (e.g., one, two, three or all four) of CD73, CD105, CD44 or CD10 expressed by the in vitro differentiated cells as measured as described elsewhere herein with a cut-off value representing cells having a known osteogenic potential.

The cells having a known osteogenic potential may be cells known to have a certain degree of osteogenic potential (e.g., no osteogenic potential, a low osteogenic potential, a high osteogenic potential or a desired degree of osteogenic potential).

The degree of osteogenic potential may be expressed as the amount of bone matrix formed and/or by the rate of bone matrix formation in vitro or in vivo. The amount of bone matrix and/or the rate of bone matrix formation may be determined by any methods known in the art, such as bone histomorphometry, histology (e.g., Collagen I, Masson's trichrome Goldner) and immunofluorescence (e.g., Collagen I, tetracycline bone labelling). For example, the thickness of newly mineralized bone, the surface area of the neo-formed bone, or the presence of at least one mineralized nodule may be evaluated in vivo after administration of the cells to mice by subcutaneous injection.

For instance, the degree of osteogenic potential of cells can be determined by measuring osteogenic activity of such cells. The osteogenic activity of human in vitro differentiated cells can be measured in vivo for example by determining the presence of at least one mineralized nodule (e.g., of human or mixed human-murine origin) after administration of the cells to mice by subcutaneous injection over the calvaria. The osteogenic activity of human in vitro differentiated cells can be measured in vivo for example by evaluating the thickness of newly mineralized nodules (e.g., of human or mixed human-murine origin) after administration of the cells to mice by subcutaneous injection over the calvaria, or by evaluating the degree of bone repair in a mouse model of femoral segmental sub-critical size defect (sub-CSD).

For example, a quantity of human cells, such as of 2.5×10⁶ cells formulated in 100 μl excipient, can be administered to nude mice by a single subcutaneous administration over the calvaria bone. To label bone neo-formation over time, calcium-binding fluorochromes such as alizarin red (red), calcein (green), calcein (blue) and tetracycline (yellow) can be sequentially administered to mice intraperitoneal injection 3 days before and 4, 8, and 12 days after cell administration of the cells, respectively. Mice can be euthanized 2 weeks after cell administration and the calvaria of each mouse can be harvested to assess bone formation properties by histomorphometry (e.g., quantification of bone formation). The initial and final thicknesses of the calvaria can be used to calculate the percentage of neo-bone formation following administration of the cells. Furthermore, bone formation properties can also be assessed by immunofluorescence (e.g., murine or human origin of the bone formation). Osteoblastic activity can be assessed on calvaria sections using ALP enzymatic activity detection method. Osteoclastic activity can be assessed on calvaria sections using TRAP enzymatic activity detection methods. The status of mineralization of the neo-formed bone can be assessed using Masson Trichrome Goldner staining on the calvaria sections stained with ALP for instance using commercially available kits (e.g., Bio-Optica®). Cartilage formation can be assessed using safranin-orange staining on calvaria sagittal paraffin sections.

In a further example, human in vitro differentiated cells, such as of 1.25×10⁶ cells formulated in 50 μl excipient, can be administered to mice locally at the site of the bone defect by percutaneous injection one day after they were subjected to the femoral segmental sub-critical size defect. Bone repair can be quantified by X-ray imaging. The bone defect size can be quantified by measuring the distance between the two edges of the bone defect.

In particular embodiments, the cells having a known osteogenic potential may be cells known to form bone matrix without the need of a calcified cartilage matrix as template (e.g., cells known to form bone by intramembranous ossification) or cells known to form bone matrix by first forming a calcified cartilage matrix and subsequently using said calcified cartilage matrix as a template for bone tissue formation (e.g., cells known to form bone by endochondral ossification). The type of bone formation (e.g., endochondral ossification or intramembranous ossification) may be determined by any methods known in the art, such as bone histomorphometry, histology (e.g., Collagen I, Masson's trichrome Goldner, safranin-orange, SOX9, Collagen type II) and immunofluorescence (e.g., Collagen I, tetracycline bone labelling, Collagen type II). For example, the presence of at least one mineralized nodule after administration of the cells to mice by subcutaneous injection as described elsewhere herein may indicate that bone matrix is formed by endochondral ossification.

In particular embodiments, the cells having a known osteogenic potential are cells known to form bone by endochondral ossification. For instance, the cells having a known osteogenic potential may be human cells that form at least one mineralized nodule of human origin in vivo after administration of the cells to mice by subcutaneous injection, preferably wherein the subcutaneous injection is performed over the calvaria bone.

In particular embodiments, the cells having a known osteogenic potential are cells (e.g., human cells) that show an increase in in vivo bone formation (e.g., of human origin) by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more) after administration of the cells, e.g., to mice by subcutaneous injection, relative to the bone formation observed when control cells (e.g., cells with no osteogenic potential or cells with a low osteogenic potential) or vehicle are administered, e.g., to mice by subcutaneous injection, preferably wherein the subcutaneous injection is performed over the calvaria bone. For example, undifferentiated MSCs obtained from the same donor as the cells having a known osteogenic potential may be used as control cells.

Non-limiting examples of cells known to have a low osteogenic potential include in vitro differentiated MSC-derived cells (referred to herein as “Cell product A” or “bone-forming cells A”) obtained as follows: bone marrow white blood cells are seeded at a density of 50,000 cells/cm² in the culture medium, and incubated at 37° C. in a humidified incubator containing 5% CO₂. 4 days after cell seeding, non-adherent cells are removed and the medium is renewed with conventional culture medium supplemented with 5% Octaserum (50:50 autologous serum and OctaPlasLG® (Octapharma)), FGF-b (CellGenix), TGFβ-1 (Humanzyme). 7 days and 11 days after seeding, half of the culture medium is removed and replaced with fresh one. Cells are cultured during primary culture for 14 days. At day 14, cells are harvested by detachment, e.g., with Trypzean (Lonza), and by swirling and pipetting up and down (passage 1: P1). The intermediate cells are cryopreserved in freezing medium comprising culture medium, 10% Octaserum (50:50 autologous serum and OctaPlasLG® (Octapharma)), 10% DMSO) and stored in liquid nitrogen. For secondary culture, cells are thawed and re-plated at a density of 1144 cells/cm². Cells are cultured during secondary culture for 14 days. At day 28, cells are harvested by detachment, e.g., with Trypzean (Lonza), and by swirling and pipetting up and down (passage 2: P2). To obtain the final cell product, cells are resuspended, e.g., in OctaPlasLG®, at a final concentration of 25×10⁶ cells/ml. This cell product is referred to herein as “Cell product A” or “bone-forming cells A”.

Non-limiting examples of cells known to have a high osteogenic potential include in vitro differentiated MSC-derived cells (referred to herein as “Cell product B” or “bone-forming cells B”) obtained as follows: bone marrow white blood cells are seeded at a density of 50,000 cells/cm² in a conventional culture medium comprising 5% OctaPlasLG® (Octapharma), 0.1 UI/ml Heparin (LEO Pharma), FGF-b (CellGenix), TGFβ-1 (Humanzyme).), and incubated at 37° C. in a humidified incubator containing 5% CO₂. 4 days after cell seeding, non-adherent cells are removed and the medium is renewed with culture medium. 7 days and 11 days after seeding, half of the culture medium is removed and replaced with fresh one to renew growth factors. Cells are cultured during primary culture for 14 days. At day 14, cells are harvested by detachment, e.g., with Trypzean (Lonza), and by swirling and pipetting up and down (passage 1: P1). The intermediate cells are cryopreserved (e.g., in CryoStor® CS10 (BioLife Solutions)) and stored in liquid nitrogen. Next, intermediate cells are thawed and re-plated for secondary culture at a density of 286 cells/cm². Cells are cultured during secondary culture for 14 days. At day 28, cells are harvested by detachment, e.g., with Trypzean (Lonza), and by swirling and pipetting up and down (passage 2: P2). To obtain the final cell product, cells are resuspended, e.g., in OctaPlasLG®, at a final concentration of 25×10⁶ cells/ml. This cell product is referred to herein as “Cell product B” or “bone-forming cells B”.

In particular embodiments, the cells having a known osteogenic potential are cells having a clinically useful osteogenic potential.

In certain embodiments, the cut-off value of (b1) and/or said respective cut-off values of (b2) may be cut-off values representing cells having clinically useful osteogenic potential.

The term “clinically useful” when used in relation to osteogenic potential of cells refers to a degree of osteogenic potential of cells allowing the cells to form upon transplantation of the cells in a subject bone matrix in an amount and/or by a mechanism (e.g., endochondral ossification or intramembranous ossification) that is therapeutically meaningful for a subject, such as that provides for a clinically relevant benefit to the subject, such as a subject suffering from a musculoskeletal disease or a bone-related disorder.

In certain embodiments, the in vitro differentiated cells may have clinically useful osteogenic potential if at least 50% of the animals (e.g., mice) form mineralized nodules (e.g., of human or mixed human-murine origin) after administration of the cells to the animals (e.g., mice) by subcutaneous injection over the calvaria. In certain embodiments, the in vitro differentiated cells may have clinically useful osteogenic potential if at least 60%, at least 65% at least 70%, at least 75%, at least 80%, at least 85%, at least 90, or at least 95% of the animals (e.g., mice) form mineralized nodules (e.g., of human or mixed human-murine origin) after administration of the cells to the animals (e.g., mice) by subcutaneous injection over the calvaria.

Non-limiting examples of musculoskeletal diseases may include local or systemic disorders, such as, any type of osteoporosis or osteopenia, e.g., primary, postmenopausal, senile, corticoid-induced, bisphosphonates-induced, and radiotherapy-induced; any secondary, mono- or multisite osteonecrosis; any type of fracture, e.g., non-union, mal-union, delayed union fractures or compression, maxillo-facial fractures; conditions requiring bone fusion (e.g., spinal fusions and rebuilding); congenital bone defect; bone reconstruction, e.g., after traumatic injury or cancer surgery, and cranio-facial bone reconstruction; traumatic arthritis, focal cartilage and/or joint defect, focal degenerative arthritis; osteoarthritis, degenerative arthritis, gonarthrosis, and coxarthrosis; osteogenesis imperfecta; osteolytic bone cancer; Paget's Disease; endocrinological disorders; hypophosphatemia; hypocalcemia; renal osteodystrophy; osteomalacia; adynamic bone disease, hyperparathyroidism, primary hyperparathyroidism, secondary hyperparathyroidism; periodontal disease; Gorham-Stout disease and McCune-Albright syndrome; rheumatoid arthritis; spondyloarthropathies, including ankylosing spondylitis, psoriatic arthritis, enteropathic arthropathy, and undifferentiated spondyloarthritis and reactive arthritis; systemic lupus erythematosus and related syndromes; scleroderma and related disorders; Sjogren's Syndrome; systemic vasculitis, including Giant cell arteritis (Horton's disease), Takayasu's arteritis, polymyalgia rheumatica, ANCA-associated vasculitis (such as Wegener's granulomatosis, microscopic polyangiitis, and Churg-Strauss Syndrome), Behcet's Syndrome, and other polyarteritis and related disorders (such as polyarteritis nodosa, Cogan's Syndrome, and Buerger's disease); arthritis accompanying other systemic inflammatory diseases, including amyloidosis and sarcoidosis; crystal arthropathies, including gout, calcium pyrophosphate dihydrate disease, disorders or syndromes associated with articular deposition of calcium phosphate or calcium oxalate crystals; chondrocalcinosis and neuropathic arthropathy; Felty's Syndrome and Reiter's Syndrome; Lyme disease and rheumatic fever.

By way of example, but not limitation, bone-related disorders which can benefit from transplantation of cells having a clinically useful osteogenic potential may include local or systemic disorders, such as, any type of osteoporosis or osteopenia, e.g., primary, postmenopausal, senile, corticoid-induced, any secondary, mono- or multisite osteonecrosis, any type of fracture, e.g., non-union, mal-union, delayed union fractures or compression, conditions requiring bone fusion (e.g., spinal fusions and rebuilding), maxillo-facial fractures, bone reconstruction, e.g., after traumatic injury or cancer surgery, cranio-facial bone reconstruction, osteogenesis imperfecta, osteolytic bone cancer, Paget's Disease, endocrinological disorders, hypophosphatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

Non-limiting examples of cells having a known clinically useful osteogenic potential include “Cell product B” or “bone-forming cells B” obtained as described elsewhere herein. Further non-limiting examples of cells having a known clinically useful osteogenic potential include “Cell product C” or “bone-forming cells C” obtained as described elsewhere herein.

In particular embodiments, the cells having a known clinically useful osteogenic potential are “Cell product B” or “bone-forming cells B” obtained as described elsewhere herein. In particular embodiments, the cells having a known clinically useful osteogenic potential are “Cell product C” or “bone-forming cells C” obtained as described elsewhere herein, including “Cell product C—cryo” or “bone-forming cells C cryo(preserved)”.

In particular embodiments, the reference value for the quantity of in vitro differentiated cells expressing any one or more of CD73, CD105, CD10 or CD44 may be determined by determining the quantity of in vitro differentiated cells expressing any one or more of CD73, CD105, CD10 or CD44, respectively, in reference cells (e.g., cells known to have a clinically useful osteogenic potential), thereby producing a reference value. Similarly, the reference value for the quantity of any one or more of CD73, CD105 or CD44 expressed by the in vitro differentiated cells may be determined by determining the quantity of any one or more of CD73, CD105 or CD44, respectively, in reference cells (e.g., cells known to have a clinically useful osteogenic potential), thereby producing a reference value.

One or more reference values obtained from one or more reference cell types may be used to determine a threshold or cut-off value as generally known in the art to provide for a certain degree of osteogenic potential of cells, preferably for a clinically useful osteogenic potential.

In particular embodiments, the reference values or cut-off values in (b1) comparing the quantity (or fraction) of the in vitro differentiated cells expressing any one or more of CD73, CD105, CD10 or CD44, preferably all of CD73, CD105, CD10 and CD44, as measured as described elsewhere herein with a reference value or cut-off value representing cells having a known osteogenic potential; and/or (b2) comparing the quantity of any one or more of CD73, CD105 or CD44, preferably all of CD73, CD105 and CD44, expressed by the in vitro differentiated cells as measured as described elsewhere herein with a reference value or cut-off value representing cells having a known osteogenic potential are reference values or cut-off values representing cells having clinically useful osteogenic potential.

By extensive research, the present inventors have found that at least 90% of a cell population of in vitro differentiated cells with osteogenic potential, and especially of a cell population of in vitro differentiated cells that have a high osteogenic potential and form in vivo bone matrix by endochondral ossification, express any one or more of CD73, CD105, CD10 or CD44, preferably all of CD73, CD105, CD10 and CD44. Furthermore, these in vitro differentiated cells also express an increased quantity of CD73 and/or CD44 and a decreased quantity of CD105 compared to the quantities of CD73, CD44 and CD105, respectively, in MSCs.

Accordingly, in particular embodiments, the method as taught herein comprises (c1) finding a deviation or no deviation of the quantity (or fraction) of the in vitro differentiated cells expressing any one or more of CD73, CD105, CD10 or CD44, preferably all of CD73, CD105, CD10 and CD44, as measured as described elsewhere herein from the reference value or cut-off value representing cells having a known osteogenic potential, and/or (c2) finding a deviation or no deviation of the quantity of any one of CD73, CD105 or CD44, preferably all of CD73, CD105 and CD44, expressed by the in vitro differentiated cells as measured as described elsewhere herein from the reference value or the cut-off value representing cells having a known osteogenic potential, and (d) attributing the deviation or no deviation found in (c1) and/or (c2) to a particular determination of osteogenic potential of the in vitro differentiated cells. Hence, also provided herein is the method as taught herein comprising (c1) finding a deviation or no deviation of the quantity (or fraction) of the in vitro differentiated cells expressing all of CD73, CD105, CD10 and CD44 as measured as described elsewhere herein from the reference value or cut-off value representing cells having a known osteogenic potential, and/or (c2) finding a deviation or no deviation of the quantity of all of CD73, CD105 and CD44 expressed by the in vitro differentiated cells as measured as described elsewhere herein from the reference value or the cut-off value representing cells having a known osteogenic potential, and (d) attributing the deviation or no deviation found in (c1) and/or (c2) to a particular determination of osteogenic potential of the in vitro differentiated cells.

In certain embodiments, the methods as taught herein may comprise (c1) finding a deviation or no deviation of the quantity (or fraction) of the in vitro differentiated cells expressing CD73, CD105, CD10 and CD44, as measured as described elsewhere herein from the reference value or cut-off value representing cells having a known osteogenic potential, and/or (c2) finding a deviation or no deviation of the quantity of any one (e.g., one, two or all three) of CD73, CD105 or CD44; preferably all of CD73, CD105 and CD44; expressed by the in vitro differentiated cells as measured as described elsewhere herein from the reference value or the cut-off value representing cells having a known osteogenic potential, and (d) attributing the deviation or no deviation found in (c1) and/or (c2) to a particular determination of osteogenic potential of the in vitro differentiated cells.

In certain embodiments, the methods as taught herein may comprise (c1) finding a deviation or no deviation of the quantity (or fraction) of the in vitro differentiated cells expressing CD73, CD105, CD10 and CD44, as measured as described elsewhere herein from the reference value or cut-off value representing cells having a known osteogenic potential, and/or (c2) finding a deviation or no deviation of the quantity of any one (e.g., one, two, three or all four) of CD73, CD105, CD44 or CD10; preferably all of CD73, CD105, CD44 and CD10; expressed by the in vitro differentiated cells as measured as described elsewhere herein from the reference value or the cut-off value representing cells having a known osteogenic potential, and (d) attributing the deviation or no deviation found in (c1) and/or (c2) to a particular determination of osteogenic potential of the in vitro differentiated cells.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration, which falls outside of error margins of reference values in given reference cells (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in given reference cells (for example, outside of a range which comprises ≥40%, ≥50%, ≥60%, ≥70%, ≥75% or ≥80% or ≥85% or ≥90% or ≥95% or even ≥100% of values in given reference cells).

In a further embodiment, a deviation may be concluded if an observed alteration is beyond a given threshold or cut-off. For example, a deviation may be concluded if an observed alteration is below, the same or above a given threshold or cut-off.

In particular embodiments, the method as taught herein comprises, consists essentially of, or consists of:
(a1) measuring the fraction of the in vitro differentiated cells expressing any one or more of CD73, CD105, CD10 or CD44 on the cell surface of the in vitro differentiated cells;
(a2) measuring the quantity of any one or more of CD73, CD105 or CD44 on the cell surface of the in vitro differentiated cells;
(b1) comparing the fraction of the in vitro differentiated cells expressing any one or more of CD73, CD105, CD10 or CD44 as measured in (a1) with a cut-off value representing cells having a known osteogenic potential;
(b2) comparing the quantity of any one or more of CD73, CD105 or CD44 as measured in (a2) with one or more respective cut-off values representing cells having a known osteogenic potential;
(c1) finding a deviation or no deviation of the fraction of the in vitro differentiated cells expressing any one or more of CD73, CD105, CD10 or CD44 as measured in (a1) from said cut-off value;
(c2) finding a deviation or no deviation of the quantity of any one of CD73, CD105 or CD44 as measured in (a2) from said cut-off value; and
(d) attributing said deviation or no deviation to a particular determination of osteogenic potential of the in vitro differentiated cells.

Hence, also provided herein is the method as taught herein comprising, consisting essentially of, or consisting of:
(a1) measuring the fraction of the in vitro differentiated cells expressing CD73, CD105, CD10 and CD44 on the cell surface of the in vitro differentiated cells;
(a2) measuring the quantity of CD73, CD105 and CD44 on the cell surface of the in vitro differentiated cells;
(b1) comparing the fraction of the in vitro differentiated cells expressing CD73, CD105, CD10 and CD44 as measured in (a1) with a cut-off value representing cells having a known osteogenic potential;
(b2) comparing the quantity of CD73, CD105 and CD44 as measured in (a2) with one or more respective cut-off values representing cells having a known osteogenic potential;
(c1) finding a deviation or no deviation of the fraction of the in vitro differentiated cells expressing CD73, CD105, CD10 and CD44 as measured in (a1) from said cut-off value;
(c2) finding a deviation or no deviation of the quantity of CD73, CD105 and CD44 as measured in (a2) from said cut-off value; and
(d) attributing said deviation or no deviation to a particular determination of osteogenic potential of the in vitro differentiated cells.

In particular embodiments, wherein the reference cells are cells known to not have a clinically useful osteogenic potential,
the same or decreased quantity or fraction of the in vitro differentiated cells expressing any one or more of CD73, CD105, CD10 or CD44 as measured as described elsewhere herein compared with the cut-off value cells known to have no clinically useful osteogenic potential as described elsewhere herein indicates that the in vitro differentiated cells have no clinically useful osteogenic potential; or
an increased quantity or fraction of the in vitro differentiated cells expressing any one or more of CD73, CD105, CD10 or CD44 as measured as described elsewhere herein compared with the cut-off value representing cells known to have no clinically useful osteogenic potential as described elsewhere herein indicates that the in vitro differentiated cells have clinically useful osteogenic potential, and/or
the same or a decreased quantity of CD73, CD44 and/or CD10 expressed by the in vitro differentiated cells as measured as described elsewhere herein compared with the respective cut-off values of representing cells known to have no clinically useful osteogenic potential as described elsewhere herein, and the same or an increased quantity of CD105 expressed by the in vitro differentiated cells as measured as described elsewhere herein compared with the respective cut-off value representing known to have no clinically useful osteogenic potential as described elsewhere herein indicates that the in vitro differentiated cells have no clinically useful osteogenic potential; or
an increased quantity of any one of CD73, CD44 and/or CD10 expressed by the in vitro differentiated cells as measured as described elsewhere herein compared with the respective cut-off values representing cells known to have no clinically useful osteogenic potential as described elsewhere herein, and/or a decreased quantity of CD105 expressed by the in vitro differentiated cells as measured as described elsewhere herein compared with the respective cut-off value of representing cells known to have no clinically useful osteogenic potential as described elsewhere herein indicates that the in vitro differentiated cells have clinically useful osteogenic potential,
   preferably wherein the expression of any one or more of CD73, CD105, CD10 or CD44 represents the expression of CD73, CD105, CD10 or CD44, respectively, on the cell surface.

In particular embodiments, wherein the reference cells are cells known to have a desired osteogenic potential, preferably a clinically useful osteogenic potential,
a decreased quantity or fraction of the in vitro differentiated cells expressing any one or more of CD73, CD105, CD10 or CD44 as measured as described elsewhere herein compared with the cut-off value representing cells known to have a desired osteogenic potential as described elsewhere herein indicates that the in vitro differentiated cells do not have the desired osteogenic potential, or
the same or an increased quantity or fraction of the in vitro differentiated cells expressing any one or more of CD73, CD105, CD10 or CD44 as measured as described elsewhere herein compared with the cut-off value cells known to have a desired osteogenic potential as described elsewhere herein indicates that the in vitro differentiated cells have the desired osteogenic potential, preferably a clinically useful osteogenic potential; and/or
a decreased quantity of any one of CD73, CD44 and/or CD10 expressed by the in vitro differentiated cells as measured as described elsewhere herein compared with the respective cut-off values representing cells known to have a desired osteogenic potential as described elsewhere herein, and/or an increased quantity of CD105 expressed by the in vitro differentiated cells as measured as described elsewhere herein compared with the respective cut-off value of representing cells known to have a desired osteogenic potential as described elsewhere herein indicates that the in vitro differentiated cells do not have the desired osteogenic potential, or
the same or an increased quantity of CD73, CD44 and/or CD10 expressed by the in vitro differentiated cells as measured as described elsewhere herein compared with the respective cut-off values of representing cells known to have a desired osteogenic potential as described elsewhere herein, and the same or a decreased quantity of CD105 expressed by the in vitro differentiated cells as measured as described elsewhere herein compared with the respective cut-off value representing cells known to have a desired osteogenic potential as described elsewhere herein indicates that the in vitro differentiated cells have the desired osteogenic potential, preferably a clinically useful osteogenic potential,
   preferably wherein the expression of any one or more of CD73, CD105, CD10 or CD44 represents the expression of CD73, CD105, CD10 or CD44, respectively, on the cell surface.

In particular embodiments, wherein the reference cells are cells known to not have a clinically useful osteogenic potential,
the same or a decreased quantity of CD10 expressed by the in vitro differentiated cells as measured as described elsewhere herein compared with the respective cut-off values of representing cells known to have no clinically useful osteogenic potential as described elsewhere herein indicates that the in vitro differentiated cells have no clinically useful osteogenic potential; or
an increased quantity of CD10 expressed by the in vitro differentiated cells as measured as described elsewhere herein compared with the respective cut-off values representing cells known to have no clinically useful osteogenic potential as described elsewhere herein indicates that the in vitro differentiated cells have clinically useful osteogenic potential,
   preferably wherein the expression of CD10 represents the expression of CD10 on the cell surface.

In particular embodiments, wherein the reference cells are cells known to have a desired osteogenic potential, preferably a clinically useful osteogenic potential,
a decreased quantity of CD10 expressed by the in vitro differentiated cells as measured as described elsewhere herein compared with the respective cut-off values representing cells known to have a desired osteogenic potential as described elsewhere herein indicates that the in vitro differentiated cells do not have the desired osteogenic potential, or
the same or an increased quantity of CD10 expressed by the in vitro differentiated cells as measured as described elsewhere herein compared with the respective cut-off values of representing cells known to have a desired osteogenic potential as described elsewhere herein indicates that the in vitro differentiated cells have the desired osteogenic potential, preferably a clinically useful osteogenic potential,
   preferably wherein the expression of CD10 represents the expression of CD10 on the cell surface.

Certain embodiments provide the methods as taught herein, wherein:
the same or an increased fraction of the in vitro differentiated cells as measured in (a1) compared with the cut-off value of (b1) indicates that the in vitro differentiated cells have clinically useful osteogenic potential; and
the same or an increased quantity of CD73, CD44 and/or CD10 as measured in (a2) compared with the respective cut-off values of (b2), and the same or a decreased quantity of CD105 as measured in (a2) compared with the respective cut-off value of (b2) indicates that the in vitro differentiated cells have clinically useful osteogenic potential.

In certain embodiments, said cut-off value of (b1) is 90% of in vitro differentiated cells expressing CD73, CD105, CD10 and CD44 on the cell surface; and wherein said cut-off value of (b2) is a normalized Median of Fluorescence Intensity (nMFI) for CD73 of 500, a nMFI for CD44 of 100, a nMFI for CD105 of 150 and/or a nMFI for CD10 of 40. In certain embodiments, said cut-off value of (b1) is 90% of in vitro differentiated cells expressing CD73, CD105, CD10 and CD44 on the cell surface; and wherein said cut-off value of (b2) is a nMFI for CD73 of 500, a nMFI for CD44 of 100, a nMFI for CD105 of 150 and/or a nMFI for CD10 of 50.

In certain embodiments, said cut-off value of (b1) is 90% of in vitro differentiated cells expressing CD73, CD105, CD10 and CD44 on the cell surface; and wherein said cut-off value of (b2) is a normalized nMFI for CD73 of 500, a nMFI for CD44 of 150, a nMFI for CD105 of 150 and/or a nMFI for CD10 of 40. In certain embodiments, said cut-off value of (b1) is 90% of in vitro differentiated cells expressing CD73, CD105, CD10 and CD44 on the cell surface; and wherein said cut-off value of (b2) is a normalized nMFI for CD73 of 500, a nMFI for CD44 of 150, a nMFI for CD105 of 150 and/or a nMFI for CD10 of 50.

In certain embodiments, the quantity of CD73, CD105 and CD44 expressed by the in vitro differentiated cells is measured. In certain embodiments, said cut-off value of (b2) is a nMFI for CD73 of 500, a nMFI for CD44 of 100 and a nMFI for CD105 of 150.

In certain embodiments, the quantity of CD73, CD105, CD44 and CD10 expressed by the in vitro differentiated cells is measured. In certain embodiments, said cut-off value of (b2) is a nMFI for CD73 of 500, a nMFI for CD44 of 100, a nMFI for CD105 of 150, and a nMFI for CD10 of 40. In certain embodiments, said cut-off value of (b2) is a nMFI for CD73 of 500, a nMFI for CD44 of 150, a nMFI for CD105 of 150, and a nMFI for CD10 of 40. In certain embodiments, said cut-off value of (b2) is a nMFI for CD73 of 500, a nMFI for CD44 of 100, a nMFI for CD105 of 150, and a nMFI for CD10 of 40. In certain embodiments, said cut-off value of (b2) is a nMFI for CD73 of 500, a nMFI for CD44 of 150, a nMFI for CD105 of 150, and a nMFI for CD10 of 50.

In particular embodiments, the cut-off value of (b1) comparing the fraction of the in vitro differentiated cells expressing any one or more of CD73, CD105, CD10 or CD44 with a cut-off value representing cells having a known osteogenic potential, preferably a known clinical useful osteogenic potential, is 90%, 91%, 92%, 93%, 94%, 95%; 96%; 97%, 98% or 99%, preferably 90%, of in vitro differentiated cells expressing any one or more of CD73, CD105, CD10 or CD44, preferably wherein the expression of any one or more of CD73, CD105, CD10 or CD44 represents the expression of CD73, CD105, CD10 or CD44, respectively, on the cell surface. Hence, provided herein is the cut-off value of (b1) comparing the fraction of the in vitro differentiated cells expressing all of CD73, CD105, CD10 and CD44 with a cut-off value representing cells having a known osteogenic potential, preferably a known clinical useful osteogenic potential, is 90%, 91%, 92%, 93%, 94%, 95%; 96%; 97%, 98% or 99%, preferably 90%, of in vitro differentiated cells expressing all of CD73, CD105, CD10 and CD44, preferably wherein the expression of all of CD73, CD105, CD10 and CD44 represents the expression of CD73, CD105, CD10 and CD44, respectively, on the cell surface. In a particular embodiment, if about 90%, 91%, 92%, 93%, 94%, 95%; 96%; 97%, 98% or 99%, preferably 90%, or more of the in vitro differentiated cells express any one or more of CD73, CD105, CD10 or CD44, preferably all of CD73, CD105, CD10 and CD44, the in vitro differentiated cells have the desired osteogenic potential, preferably a clinically useful osteogenic potential. Hence, also provided herein is that if about 90% or more of the in vitro differentiated cells express all of CD73, CD105, CD10 and CD44, the in vitro differentiated cells have the desired osteogenic potential, preferably a clinically useful osteogenic potential.

In particular embodiments, the cut-off value of (b2) comparing the quantity of any one or more of CD73, CD105, CD44 and/or CD10 expressed by the in vitro differentiated cells on the cell surface with a cut-off value representing cells having a known osteogenic potential, preferably a known clinical useful osteogenic potential, is a nMFI_{CD73} of 500, 550, 600, 650, 700, 750, 800, 850 or 900, preferably a nMFI_{CD73} of 500, a nMFI_{CD44} of 100, 110, 120, 130, 140, 150, 200, 250, 300 or 350, preferably a nMFI_{CD44} of 100, a nMFI_{CD105} of 180, 170, 160, 150, 140, 130, 120, 110 or 100, preferably a nMFI_{CD105} of 150 and/or a nMFI_{CD10} of 10, 15, 20, 25, 30, 35, 40, 45, 50, 55 or 60, preferably a nMFI_{CD10} of 50; preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC, and/or the nMFI_{CD10} is measured with an excitation wavelength of 488 nm and an emission wavelength of 580 nm for PE.

Hence, also provided herein is the cut-off value of (b2) comparing the quantity of CD73, CD105 and CD44 expressed by the in vitro differentiated cells on the cell surface with a cut-off value representing cells having a known osteogenic potential, preferably a known clinical useful osteogenic potential, is a nMFI_{CD73} of 500, 550, 600, 650, 700, 750, 800, 850 or 900, preferably a nMFI_{CD73} of 500, a nMFI_{CD44} of 100, 110, 120, 130, 140, 150, 200, 250, 300 or 350, preferably a nMFI_{CD44} of 100 and a nMFI_{CD105} of 180, 170, 160, 150, 140, 130, 120, 110 or 100, preferably a nMFI_{CD105} of 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and the nMFI₁₀₅ is measured with an excitation wavelength of 633 nm and an emission wavelength of 660 nm for APC.

In certain embodiments, the cut-off value of (b2) comparing the quantity of any one or more of CD73, CD105, CD44 and CD10 expressed by the in vitro differentiated cells on the cell surface with a cut-off value representing cells having a known osteogenic potential, preferably a known clinical useful osteogenic potential, is a nMFI_{CD73} of 500, 550, 600, 650, 700, 750, 800, 850 or 900, preferably a nMFI_{CD73} of 500, a nMFI_{CD44} of 100, 110, 120, 130, 140, 150, 200, 250, 300 or 350, preferably a nMFI_{CD44} of 100, a nMFI_{CD105} of 180, 170, 160, 150, 140, 130, 120, 110 or 100, preferably a nMFI_{CD105} of 150 and a nMFI_{CD10} of 10, 15, 20, 25, 30, 35, 40, 45, 50, 55 or 60, preferably a nMFI_{CD10} of 50; preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC, and the nMFI_{CD10} is measured with an excitation wavelength of 488 nm and an emission wavelength of 580 nm for PE.

In particular embodiments, the cut-off value of (b2) comparing the quantity of CD10 expressed by the in vitro differentiated cells on the cell surface with a cut-off value representing cells having a known osteogenic potential is a nMFI_{CD10} of 10, 15, 20, 25, or 30, preferably a nMFI_{CD10} of 20. In particular embodiments, the cut-off value of (b2) comparing the quantity of CD10 expressed by the in vitro differentiated cells on the cell surface with a cut-off value representing cells having a clinically useful osteogenic potential is a nMFI_{CD10} of 40, 45, 50, 55, or 60, preferably a nMFI_{CD10} of 40; more preferably a nMFI_{CD10} of 50; even more preferably a nMFI_{CD10} of 60. Preferably, the nMFI_{CD10} is measured with an excitation wavelength of 488 nm and an emission wavelength of 580 nm for PE.

The recitations “a nMFI for CD73” or “nMFI_{CD73}” as used herein refers to the ratio of the MFI of the whole analyzed cell population labeled with an APC-conjugated antibody against CD73 (e.g., BD Biosciences®, Cat No: 560847) to the MFI of the cell population labeled with IgG control conjugated with APC (e.g., BD Biosciences®, Cat No: 555751). Preferably, the nMFI_{CD73} is measured with an excitation wavelength of 633 nm and an emission wavelength of 660 nm for APC.

The recitations “a nMFI for CD44” or “nMFI_{CD44}” as used herein refers to the ratio of the MFI of the whole analyzed cell population labeled with PE-conjugated antibody against CD44 (e.g., BD Biosciences®, Cat No: 550989) to the MFI of the cell population labeled with IgG control conjugated with PE (e.g., BD Biosciences®, Cat No: 556650). Preferably, the nMFI_{CD44} is measured with an excitation wavelength of 488 nm and an emission wavelength of 580 nm for PE.

The recitation “a nMFI for CD105” or “nMFI_{CD105}” as used herein refers to the ratio of the MFI of the whole analyzed cell population labeled with APC-conjugated antibodies against CD105 (e.g., BD Biosciences®, Cat No: 562408) to the MFI of the cell population labeled with IgG control conjugated with APC (e.g., BD Biosciences®, Cat No: 555751). Preferably, the nMFI₁₀₅ is measured with an excitation wavelength of 633 nm and an emission wavelength of 660 nm for APC.

The recitations “a nMFI for CD10” or “nMFI_{CD10}” as used herein refers to the ratio of the MFI of the whole analyzed cell population labeled with PE-conjugated antibody against CD10 (e.g., BD Biosciences®, Cat No: 555375) to the MFI of the cell population labeled with IgG control conjugated with PE (e.g., BD Biosciences®, Cat No: 556650). Preferably, the nMFI_{CD10} is measured with an excitation wavelength of 488 nm and an emission wavelength of 580 nm for PE.

In particular embodiments, a nMFI_{CD73} of 500, 550, 600, 650, 700, 750, 800, 850 or 900, preferably 500, or more expressed by the in vitro differentiated cells as measured as described elsewhere herein indicates that the in vitro differentiated cells have the desired osteogenic potential, preferably a clinically useful osteogenic potential.

In particular embodiments, a nMFI_{CD44} of 100, 110, 120, 130, 140, 150, 200, 250, 300 or 350, preferably 100, or more expressed by the in vitro differentiated cells as measured as described elsewhere herein indicates that the in vitro differentiated cells have the desired osteogenic potential, preferably a clinically useful osteogenic potential.

In particular embodiments, a nMFI_{CD105} of 180, 170, 160, 150, 140, 130, 120, 110 or 100 preferably 150, or less expressed by the in vitro differentiated cells as measured as described elsewhere herein indicates that the in vitro differentiated cells have the desired osteogenic potential, preferably a clinically useful osteogenic potential.

In particular embodiments, a nMFI_{CD10} of at least 10, at least 15, at least 20, at least 25, at least 30, at least 40, at least 45, at least 50, at least 55, or at least 60, preferably at least 50, expressed by the in vitro differentiated cells as measured as described elsewhere herein indicates that the in vitro differentiated cells have the desired osteogenic potential, preferably a clinically useful osteogenic potential.

In preferred embodiments, a nMFI_{CD73} of 500 or more, a nMFI_{CD44} of 100 or more, and a nMFI_{CD105} of 150 or less expressed by the in vitro differentiated cells as measured as described elsewhere herein indicates that the in vitro differentiated cells have the desired osteogenic potential, preferably a clinically useful osteogenic potential, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

In particular embodiments, the cut-off value of (b2) comparing the quantity of any one or more of CD73, CD105, CD44 and/or CD10 expressed by the in vitro differentiated cells on the cell surface with a cut-off value representing cells having a known osteogenic potential, preferably a known clinical useful osteogenic potential, is a nMFI_{CD73} of 500, a nMFI_{CD44} of 100, a nMFI_{CD105} of 150 and/or a nMFI_{CD10} of 40, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC, and/or the nMFI_{CD10} is measured with an excitation wavelength of 488 nm and an emission wavelength of 580 nm for PE.

In particular embodiments, the cut-off value of (b1) comparing the fraction of the in vitro differentiated cells expressing any one or more of CD73, CD105, CD10 or CD44 on the cell surface with a cut-off value representing cells having a known osteogenic potential, preferably a known clinical useful osteogenic potential, is 90% of in vitro differentiated cells expressing any one or more of CD73, CD105, CD10 or CD44; and/or the cut-off value of (b2) comparing the quantity of CD73, CD105, CD44 and/or CD10 expressed by the in vitro differentiated cells on the cell surface with a cut-off value representing cells having a known osteogenic potential, preferably a known clinical useful osteogenic potential, is a nMFI_{CD73} of 500, a nMFI_{CD44} of 100, a nMFI_{CD105} of 150 and/or a nMFI_{CD10} of 40, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC, and/or the nMFI_{CD10} is measured with an excitation wavelength of 488 nm and an emission wavelength of 580 nm for PE.

In particular embodiments, the in vitro differentiated cells are obtained or derived from pluripotent stem cells (PS), such as mammalian PS and human PS, preferably human PS.

The term “in vitro differentiated cells” as used throughout the specification refers to any cells which were cultured in vitro under appropriate conditions allowing cells to transition from one cell type into another. Differentiation of cells may involve culturing MSC under conditions capable of inducing the differentiation of cells towards the desired cell type, more typically culturing cells in a medium comprising one or more agents (e.g., growth factors) capable of inducing the differentiation of cells towards the desired cell type. The term “stem cell” refers generally to an unspecialized or relatively less specialized and proliferation-competent cell, which is capable of self-renewal, i.e., can proliferate without differentiation, and which or the progeny of which can give rise to at least one relatively more specialized cell type. The term encompasses stem cells capable of substantially unlimited self-renewal, i.e., wherein the progeny of a stem cell or at least part thereof substantially retains the unspecialized or relatively less specialized phenotype, the differentiation potential, and the proliferation capacity of the mother stem cell, as well as stem cells which display limited self-renewal, i.e., wherein the capacity of the progeny or part thereof for further proliferation and/or differentiation is demonstrably reduced compared to the mother cell. By means of example and not limitation, a stem cell may give rise to descendants that can differentiate along one or more lineages to produce increasingly relatively more specialized cells, wherein such descendants and/or increasingly relatively more specialized cells may themselves be stem cells as defined herein, or even to produce terminally differentiated cells, i.e., fully specialized cells, which may be post-mitotic.

Included in the definition of mPS cells are without limitation embryonic stem cells of various types, exemplified without limitation by murine embryonic stem cells, e.g., as described by Evans & Kaufman 1981 (Nature 292: 154-6) and Martin 1981 (PNAS 78: 7634-8); rat pluripotent stem cells, e.g., as described by Iannaccone et al. 1994 (Dev Biol 163: 288-292); hamster embryonic stem cells, e.g., as described by Doetschman et al. 1988 (Dev Biol 127: 224-227); rabbit embryonic stem cells, e.g., as described by Graves et al. 1993 (Mol Reprod Dev 36: 424-433); porcine pluripotent stem cells, e.g., as described by Notarianni et al. 1991 (J Reprod Fertil Suppl 43: 255-60) and Wheeler 1994 (Reprod Fertil Dev 6: 563-8); sheep embryonic stem cells, e.g., as described by Notarianni et al. 1991 (supra); bovine embryonic stem cells, e.g., as described by Roach et al. 2006 (Methods Enzymol 418: 21-37); human embryonic stem (hES) cells, e.g., as described by Thomson et al. 1998 (Science 282: 1145-1147); human embryonic germ (hEG) cells, e.g., as described by Shamblott et al. 1998 (PNAS 95: 13726); embryonic stem cells from other primates such as Rhesus stem cells, e.g., as described by Thomson et al. 1995 (PNAS 92:7844-7848) or marmoset stem cells, e.g., as described by Thomson et al. 1996 (Biol Reprod 55: 254-259).

Other types of mPS cells are also included in the term as are any cells of mammalian origin capable of producing progeny that includes derivatives of all three germ layers, regardless of whether they were derived from embryonic tissue, foetal tissue or other sources. mPS cells are preferably not derived from a malignant source. A cell or cell line is from a “non-malignant source” if it was established from primary tissue that is not cancerous, nor altered with a known oncogene. It may be desirable that the mPS maintain a normal karyotype throughout prolonged culture under appropriate conditions. It may also be desirable, but not always necessary, that the mPS maintain substantially indefinite self-renewal potential under appropriate in vitro conditions.

As used herein, the qualifier “pluripotent” denotes the capacity of a cell to give rise to cell types originating from all three germ layers of an organism, i.e., mesoderm, endoderm, and ectoderm, and potentially capable of giving rise to any and all cell types of an organism, although not able of growing into the whole organism.

In more particular embodiments, the in vitro differentiated cells are obtained or derived from mesenchymal stem cells (MSC), embryonic stem cells (ESC), or induced pluripotent stem cells (iPS). In more particular embodiments of the uses or methods as taught herein, the in vitro differentiated cells are obtained or derived from MSC.

The term “mesenchymal stem cell” or “MSC” as used herein refers to an adult, mesoderm-derived stem cell that is capable of generating cells of mesenchymal lineages, typically of two or more mesenchymal lineages, more typically three or more mesenchymal lineages, e.g., chondro-osteoblastic (bone and cartilage), osteoblastic (bone), chondroblastic (cartilage), myocytic (muscle), tenocytic (tendon), fibroblastic (connective tissue), adipocytic (fat) and stromogenic (marrow stroma) lineage. MSC may be isolated from a biological sample, preferably a biological sample of a human subject, e.g., bone marrow, trabecular bone, blood, umbilical cord, placenta, foetal yolk sac, skin (dermis), specifically foetal and adolescent skin, periosteum, dental pulp, tendon and adipose tissue.

The term “biological sample” or “sample” as used herein refers to a sample obtained from a biological source, e.g., from an organism, such as an animal or human subject, cell culture, tissue sample, etc. A biological sample of an animal or human subject refers to a sample removed from an animal or human subject and comprising cells thereof. The biological sample of an animal or human subject may comprise one or more tissue types and may comprise cells of one or more tissue types. Methods of obtaining biological samples of an animal or human subject are well known in the art, e.g., tissue biopsy or drawing blood. Human MSC, their isolation, in vitro expansion, and differentiation, have been described in, e.g., U.S. Pat. Nos. 5,486,359; 5,811,094; 5,736,396; 5,837,539; or 5,827,740. Any MSC described in the art and isolated by any method described in the art may be suitable in the present method. In particular, MSC may be defined as displaying the capacity for in vitro trilineage mesenchymal differentiation into osteoblasts, adipocytes, and chondroblasts (Dominici et al., 2006, vol. 8, 315).

The term “embryonic stem cell” or “ESC” as used herein refers to pluripotent stem cells that are derived from an embryo, such as from the inner cell mass of the blastocyst, and that are capable under appropriate conditions of producing progeny of different cell types that are derivatives of all three germ layers, i.e., endoderm, mesoderm, and ectoderm, according to a standard art-accepted test, such as inter alia the ability to form a teratoma in SCID mice, or the ability to form identifiable cells of all three germ layers in tissue culture. The scope of the term “hES cells” covers pluripotent stem cells that are derived from a human embryo at the blastocyst stage, or before substantial differentiation of the cells into the three germ layers. ES cells, in particular hES cells, are typically derived from the inner cell mass of blastocysts or from whole blastocysts. Derivation of hES cell lines from the morula stage has been documented and ES cells so obtained can also be used in the invention (Strelchenko et al. 2004. Reproductive BioMedicine Online 9: 623-629). The term “induced pluripotent stem cells” or “iPS cells” as used herein refers to pluripotent stem cells generated from adult cells by reprogramming iPS cells can self-renew and can give rise to cell types originating from all three germ layers of an organism, i.e., mesoderm, endoderm, and ectoderm, and potentially to any and all cell types of an organism, although not able of growing into the whole organism. Examples of iPS cells are those as taught inter alia by Yamanaka et al. 2006 (Cell 126: 663-676) and Yamanaka et al. 2007 (Cell 131: 861-872).

The term “MSC”, “ESC”, or “iPS” also encompasses the progeny of MSC, ESC or iPS, respectively, e.g., progeny obtained by in vitro or ex vivo proliferation (propagation/expansion) of MSC, ESC or iPS, respectively, obtained from a biological sample of an animal or human subject.

The term “adult stem cell” as used herein refers to a stem cell present in or obtained from (such as isolated from) an organism at the foetal stage or preferably after birth (e.g., particularly but without limitation for a human organism, at least one month of age after birth, e.g., at least 2 months, at least 3 months, e.g., at least 4 months, at least 5 months, e.g., at least 6 months of age after birth, such as, for example, 1 year or more, 5 years or more, at least 10 years or more, 15 years or more, 20 years or more, or 25 years or more of age after birth), such as for example after achieving adulthood. By means of example, adult stem cells can be obtained from human subjects which would otherwise be described in the conventional terms “infant”, “child”, “youth”, “adolescent” or “adult”.

Except when noted, “subject”, “donor” or “patient” are used interchangeably and refer to animals, preferably vertebrates, more preferably mammals, and specifically includes human patients and non-human mammals. Preferred patients are human subjects Animal subjects include prenatal forms of animals, such as, e.g., fetuses.

In particular embodiments of the uses or methods as taught herein, the in vitro differentiated cells are human cells.

The present methods and protocols may preferably depart from pluripotent stem cell populations (e.g., mPS or hPS cell populations) which are “undifferentiated”, i.e., wherein a substantial proportion (for example, at least about 60%, preferably at least about 70%, even more preferably at least about 80%, still more preferably at least about 90% and up to 100%) of cells in the stem cell population display characteristics (e.g., morphological features and/or markers) of undifferentiated mPS cells, clearly distinguishing them from cells undergoing differentiation.

Undifferentiated mPS cells are generally easily recognised by those skilled in the art, and may appear in the two dimensions of a microscopic view with high nuclear/cytoplasmic ratios and prominent nucleoli, may grow as compact colonies with sharp borders. It is understood that colonies of undifferentiated cells within the population may often be surrounded by neighbouring cells that are more differentiated. Nevertheless, the undifferentiated colonies persist when the population is cultured or passaged under appropriate conditions known per se, and individual undifferentiated cells constitute a substantial proportion of the cell population. Undifferentiated mPS cells may express the stage-specific embryonic antigens (SSEA) 3 and 4, and markers detectable using antibodies designated Tra-1-60 and Tra-1-81 (Thomson et al. 1998, supra). Undifferentiated mPS cells may also typically express Nanog, Oct-4 and TERT. Undifferentiated mPS cells may also comprise expression of alkaline phosphatase (AP) (e.g., as determined by a suitable AP activity assay).

In particular embodiments, the in vitro differentiated cells are of chondro-osteoblastic lineage (bone and cartilage), osteoblastic lineage (bone), such as, e.g., osteochondroprogenitors and/or osteoprogenitors and/or pre-osteoblasts and/or osteoblasts and/or osteocytes, etc.; chondroblastic (cartilage) lineage, such as, e.g., osteochondroprogenitors and/or chondroprogenitors and/or pre-chondroblasts and/or chondroblasts and/or chondrocytes; adipogenic (fat); myogenic (muscle); tenogenic (tenocytes) lineage; fibroblastic (connective tissue) lineage, such as, e.g., fibroblasts, fibrocytes; or synovial (synovial fluid) lineage.

The term “chondro-osteoblastic lineage” as used herein in reference to the in vitro differentiated cells may refer to cells which have the ability to differentiate into cells of the osteoblastic lineage, such as osteochondroprogenitors, osteoprogenitors and/or pre-osteoblasts and/or osteoblasts and/or osteocytes, etc., or into cells of the chondroblastic lineage, such as osteochondroprogenitors, chondroprogenitors and/or pre-chondroblasts and/or chondroblasts and/or chondrocytes. The skilled person will understand that the cells will either differentiate into cells of the osteoblastic lineage (e.g., pre-osteoblasts or osteoblasts), or into cells of the chondroblastic lineage (e.g., pre-chondroblasts or chondroblasts) depending on conditions they are exposed to, such as physical factors, and/or chemical or biological components, such as growth factors.

In particular embodiments, the in vitro differentiated cells are cells of the osteoblastic lineage (e.g., osteoprogenitors, pre-osteoblasts, osteoblasts, or osteocytes) or chondroblastic lineage (chondroprogenitors and/or pre-chondroblasts and/or chondroblasts and/or chondrocytes).

Differentiation of MSC into cells of the chondro-osteoblast, osteoblastic or chondroblastic lineage may involve culturing MSC under conditions capable of inducing the differentiation of MSC towards cells of the chondro-osteoblast, osteoblastic or chondroblastic lineage, more typically culturing MSC in a medium comprising one or more agents (e.g., growth factors) capable of inducing the differentiation of MSC towards cells of the chondro-osteoblast, osteoblastic or chondroblastic lineage. Protocols for differentiation of MSC into cells of the chondro-osteoblast, osteoblastic or chondroblastic lineage include the process for differentiating MSC into “bone-forming cells B” as described elsewhere herein and the process for differentiating MSC into “cell product C” or “bone-forming cells C” which is as follows: bone marrow white blood cells are seeded at a density of 50,000 cells/cm² in a conventional culture medium comprising 5% OctaPlasLG® (Octapharma), 0.1 UI/ml Heparin (LEO Pharma), FGF-b (CellGenix) and TGFβ-1 (Humanzyme)), and incubated at 37° C. in a humidified incubator containing 5% CO₂. 4 days after cell seeding, non-adherent cells are removed and the medium is renewed with culture medium. 7 days and 11 days after seeding, half of the culture medium is removed and replaced with fresh one to renew growth factors. Cells are cultured during primary culture for 14 days. At day 14, cells are harvested by detachment, e.g., with Trypzean (Lonza), and by swirling and pipetting up and down (passage 1: P1). The intermediate cells are cryopreserved (e.g., in CryoStor® CS 10) and stored in liquid nitrogen. Next, intermediate cells are thawed and re-plated for secondary culture at a density of 572 cells/cm². Cells are cultured during secondary culture for 10 days. At day 24, cells are harvested by detachment, e.g., with Trypzean (Lonza), and by swirling and pipetting up and down (passage 2: P2). The intermediate cells are cryopreserved (e.g., in CryoStor® CS10) and stored in liquid nitrogen. Subsequently, intermediate cells are thawed and re-plated for tertiary culture at a density of 572 cells/cm². Cells are cultured during tertiary culture for 10 day. At day 34, cells are harvested by detachment, e.g., with Trypzean (Lonza), and by swirling and pipetting up and down (passage 3: P3). To obtain the final cell product, cells are resuspended, e.g., in OctaPlasLG®, at a final concentration of 25×10⁶ cells/ml. This cell product will be referred to herein as “Cell product C—fresh”. At the end of the tertiary culture, cells are also cryopreserved for long-time storage. Thereto, the cells are resuspended in cryopreservation medium to reach the desired concentration (25×10⁶ cells/ml). The cell suspension is then transferred into cryotubes which are stored in liquid nitrogen. This cell product will be referred to herein as “Cell product C—cryo”. The cryopreservation medium may be: CryoStor® CS10 (BioLife Solutions), or 50% (v/v) CryoStor® CS10 (BioLife Solutions Inc) and 50% (v/v) human serum albumin (Octapharma), or 95% (v/v) CryoStor® CS10 (BioLife Solutions Inc) and 5% (v/v) human serum albumin (Octapharma), or 80% (v/v) Hypothermosol® (BioLife Solutions Inc) 10% (v/v) DMSO, and 10% (v/v) human serum albumin (Octapharma)

Further protocols for differentiation of MSC into cells of the osteoblastic or chondroblastic lineage include, inter alia, WO 2009/087213; WO 2007/093431; and further REGER, R. L. et al. ‘Differentiation and Characterization of Human MSCs’. In: Mesenchymal Stem Cells: Methods and Protocols (Methods in Molecular Biology), Edited by D. J. Prockop et al Humana Press, 2008, Vol. 449, p. 93-107; VERMURI, M. C. et al. (Eds.). Mesenchymal Stem Cell Assays and Applications (Methods in Molecular Biology). Humana Press, 2011, Vol. 698, especially pages 201 to 352).

The term “growth factor” as used herein refers to a biologically active substance which influences proliferation, growth, differentiation, survival and/or migration of various cell types, and may affect developmental, morphological and functional changes in an organism, either alone or when modulated by other substances. A growth factor may typically act by binding, as a ligand, to a receptor (e.g., surface or intracellular receptor) present in cells responsive to the growth factor. A growth factor herein may be particularly a proteinaceous entity comprising one or more polypeptide chains By means of example and not limitation, the term “growth factor” encompasses the members of the fibroblast growth factor (FGF) family, bone morphogenetic protein (BMP) family, platelet-derived growth factor (PDGF) family, transforming growth factor beta (TGFβ) family, nerve growth factor (NGF) family, epidermal growth factor (EGF) family, insulin-like growth factor (IGF) family, growth differentiation factor (GDF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, and the like. The skilled person will understand that the growth factor or combination of growth factors may be any growth factor or combination of growth factors known of being capable of inducing differentiation of MSC towards a desired cell type. The skilled person will appreciate that in vitro methods for inducing differentiation of MSC towards a desired cell type (e.g., towards cells of osteochondroblastic, osteoblastic, or chondroblastic lineage) may result in a substantially pure (i.e., composed primarily of) cell population of the desired cell type. Without limitation, so-derived cell population may contain at least 90% (by number) of the desired cell type, such as, e.g., ≥91%, ≥92%, ≥93%, ≥94%, ≥95%, ≥96%, ≥97%, ≥98%, ≥99%, or 100% of the desired cell type.

The skilled person will understand that as the method as taught herein concerns the assessment of the osteogenic potential of in vitro differentiated cells, the in vitro differentiated cells as intended herein are typically cultured under conditions capable of inducing the differentiation of pluripotent cells, such as MSC, towards cells of the chondro-osteoblast, osteoblastic or chondroblastic lineage. In line therewith, the in vitro differentiated cells as intended herein are typically not cultured under conditions capable of inducing the differentiation of pluripotent cells, such as MSC, towards cells of the myocytic (muscle), tenocytic (tendon), fibroblastic (connective tissue), adipocytic (fat) or stromogenic (marrow stroma) lineage.

A further aspect provides a method for determining osteogenic potential of in vitro differentiated cells comprising, consisting essentially of or consisting of:
measuring the fraction of the in vitro differentiated cells expressing any one or more of CD73, CD105, CD10 or CD44 on the cell surface of the in vitro differentiated cells;
measuring the quantity of any one or more of CD73, CD105 or CD44 on the cell surface of the in vitro differentiated cells; and
determining that the in vitro differentiated cells have osteogenic potential if at least 90% of the in vitro differentiated cells express any one or more of CD73, CD105, CD10 or CD44, and if the in vitro differentiated cells have any one or more of a nMFI for CD73 of at least 500, a nMFI for CD44 of at least 100 or a nMFI for CD105 of at most 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

Preferably, a further aspect provides a method for determining osteogenic potential of in vitro differentiated cells comprising, consisting essentially of or consisting of:
measuring the fraction of the in vitro differentiated cells expressing CD73, CD105, CD10 and CD44 on the cell surface of the in vitro differentiated cells;
measuring the quantity of any one or more of CD73, CD105 or CD44 on the cell surface of the in vitro differentiated cells; and
determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 100 or a nMFI for CD105 of at most 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE and/or the nMFI₁₀₅ is measured with an excitation wavelength of 633 nm and an emission wavelength of 660 nm for APC.

In certain embodiments, the methods as taught herein may comprise determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 150 or a nMFI for CD105 of at most 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE and/or the nMFI₁₀₅ is measured with an excitation wavelength of 633 nm and an emission wavelength of 660 nm for APC.

In certain embodiments, the methods for determining osteogenic potential of in vitro differentiated cells may comprise, consist essentially of or consist of:
measuring the fraction of the in vitro differentiated cells expressing CD73, CD105, CD10 and CD44 on the cell surface of the in vitro differentiated cells;
measuring the quantity of any one or more of CD73, CD105, CD44 or CD10 on the cell surface of the in vitro differentiated cells; and
determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 100, a nMFI for CD105 of at most 150 or a nMFI for CD10 of at least 40, e.g., at least 50, at least 55 or at least 60, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC and/or the nMFI_{CD10} is measured with an excitation wavelength of 488 nm and an emission wavelength of 580 nm for PE.

In certain embodiments, the methods as taught herein may comprise determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 100, a nMFI for CD105 of at most 150 or a nMFI for CD10 of at least 50, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC and/or the nMFI_{CD10} is measured with an excitation wavelength of 488 nm and an emission wavelength of 580 nm for PE.

In certain embodiments, the methods as taught herein may comprise determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 150, a nMFI for CD105 of at most 150 or a nMFI for CD10 of at least 40, e.g., at least 50, at least 55 or at least 60, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC and/or the nMFI_{CD10} is measured with an excitation wavelength of 488 nm and an emission wavelength of 580 nm for PE.

In certain embodiments, the methods as taught herein may comprise determining that the in vitro differentiated cells have osteogenic potential if at least 90% of the in vitro differentiated cells express CD73, CD105, CD10 and CD44, and if the in vitro differentiated cells have any one or more of a nMFI for CD73 of at least 500, a nMFI for CD44 of at least 150, a nMFI for CD105 of at most 150 or a nMFI for CD10 of at least 50, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, the nMFI₁₀₅ is measured with an excitation wavelength of 633 nm and an emission wavelength of 660 nm for APC and/or the nMFI_{CD10} is measured with an excitation wavelength of 488 nm and an emission wavelength of 580 nm for PE.

In an aspect, the invention provides a method for determining osteogenic potential of in vitro differentiated cells, the method comprising, consisting essentially of or consisting of:
measuring the fraction of the in vitro differentiated cells expressing any one or more of CD73, CD105, CD10 or CD44 on the cell surface of the in vitro differentiated cells;
measuring the quantity of CD10 on the cell surface of the in vitro differentiated cells; and
determining that the in vitro differentiated cells have osteogenic potential if at least 90% of the in vitro differentiated cells express any one or more of CD73, CD105, CD10 or CD44, and if the in vitro differentiated cells have a nMFI for CD10 of at least 40, e.g., at least 50, at least 55 or at least 60, preferably the nMFI_{CD10} is measured with an excitation wavelength of 488 nm and an emission wavelength of 580 nm for PE.

In certain preferred embodiments, the methods as taught herein may comprise determining that the in vitro differentiated cells have osteogenic potential if at least 90% of the in vitro differentiated cells express any one or more of CD73, CD105, CD10 or CD44, and if the in vitro differentiated cells have a nMFI for CD10 of at least 50, preferably the nMFI_{CD10} is measured with an excitation wavelength of 488 nm and an emission wavelength of 580 nm for PE.

In an aspect, the invention provides a method for determining osteogenic potential of in vitro differentiated cells, the method comprising, consisting essentially of or consisting of:
measuring the fraction of the in vitro differentiated cells expressing CD73, CD105, CD10 and CD44 on the cell surface of the in vitro differentiated cells;
measuring the quantity of CD10 on the cell surface of the in vitro differentiated cells; and
determining that the in vitro differentiated cells have osteogenic potential if at least 90% of the in vitro differentiated cells express CD73, CD105, CD10 and CD44, and if the in vitro differentiated cells have a nMFI for CD10 of at least 40, e.g., at least 50, at least 55 or at least 60, preferably the nMFI_{CD10} is measured with an excitation wavelength of 488 nm and an emission wavelength of 580 nm for PE.

In certain preferred embodiments, the methods as taught herein may comprise determining that the in vitro differentiated cells have osteogenic potential if at least 90% of the in vitro differentiated cells express CD73, CD105, CD10 and CD44, and if the in vitro differentiated cells have a nMFI for CD10 of at least 50, preferably the nMFI_{CD10} is measured with an excitation wavelength of 488 nm and an emission wavelength of 580 nm for PE.

In a further aspect, the invention provides a method for determining osteogenic potential of in vitro differentiated cells, the method comprising, consisting essentially of or consisting of:
measuring the quantity of CD10 on the cell surface of the in vitro differentiated cells; and
determining that the in vitro differentiated cells have osteogenic potential if the in vitro differentiated cells have a nMFI for CD10 of at least 40, e.g., at least 50, at least 55 or at least 60, preferably the nMFI_{CD10} is measured with an excitation wavelength of 488 nm and an emission wavelength of 580 nm for PE.

In certain preferred embodiments, the methods as taught herein may comprise determining that the in vitro differentiated cells have osteogenic potential if the in vitro differentiated cells have a nMFI for CD10 of at least 50, preferably the nMFI_{CD10} is measured with an excitation wavelength of 488 nm and an emission wavelength of 580 nm for PE.

Preferably, a further aspect provides a method for determining osteogenic potential of in vitro differentiated cells comprising, consisting essentially of or consisting of:
measuring the fraction of the in vitro differentiated cells expressing CD73, CD105, CD10 and CD44 on the cell surface of the in vitro differentiated cells;
measuring the quantity of CD73, CD105 and CD44 on the cell surface of the in vitro differentiated cells; and
determining that the in vitro differentiated cells have osteogenic potential if at least 90% of the in vitro differentiated cells express CD73, CD105, CD10 and CD44, and if the in vitro differentiated cells have a nMFI for CD73 of at least 500, a nMFI for CD44 of at least 100 and a nMFI for CD105 of at most 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

In certain embodiments, the methods as taught herein may comprise determining that the in vitro differentiated cells have osteogenic potential, in particular clinically useful osteogenic potential, if at least 90%, such as at least 91%, at least 92%, at least 93%, at least 94%, at least 95%, at least 96%, at least 97%, at least 98%, at least 99% or 100%, of the in vitro differentiated cells express any one or more of CD73, CD105, CD10 or CD44, and if the in vitro differentiated cells have:
any one or more of a nMFI for CD73 of at least 500, a nMFI for CD44 of at least 100 or a nMFI for CD105 of at most 150;
a nMFI for CD73 of at least 500, a nMFI for CD44 of at least 100 and a nMFI for CD105 of at most 150;
any one or more of a nMFI_{CD73} of at least 550, at least 600, at least 650, at least 700, at least 750, at least 800, at least 850 or at least 900; a nMFI_{CD44} of at least 110, at least 120, at least 130, at least 140, at least 150, at least 200, at least 250, at least 300 or at least 350; or a nMFI_{CD105} of at most 180, at most 170, at most 160, at most 150, at most 140, at most 130, at most 120, at most 110 or at most 100;
a nMFI_{CD73} of at least 550, at least 600, at least 650, at least 700, at least 750, at least 800, at least 850 or at least 900; a nMFI_{CD44} of at least 110, at least 120, at least 130, at least 140, at least 150, at least 200, at least 250, at least 300 or at least 350; and a nMFI_{CD105} of at most 180, at most 170, at most 160, at most 150, at most 140, at most 130, at most 120, at most 110 or at most 100;
any one or more of a nMFI for CD73 of at least 700, a nMFI for CD44 of at least 200 or a nMFI for CD105 of at most 150; and/or
a nMFI for CD73 of at least 700, a nMFI for CD44 of at least 200 and a nMFI for CD105 of at most 150,
   preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

In certain embodiments, the methods as taught herein may comprise determining that the in vitro differentiated cells have osteogenic potential, in particular clinically useful osteogenic potential, if at least 90%, such as at least 91%, at least 92%, at least 93%, at least 94%, at least 95%, at least 96%, at least 97%, at least 98%, at least 99% or 100%, of the in vitro differentiated cells express CD73, CD105, CD10 and CD44, and if the in vitro differentiated cells have:
any one or more of a nMFI for CD73 of at least 500, a nMFI for CD44 of at least 100 or a nMFI for CD105 of at most 150;
a nMFI for CD73 of at least 500, a nMFI for CD44 of at least 100 and a nMFI for CD105 of at most 150;
any one or more of a nMFI_{CD73} of at least 550, at least 600, at least 650, at least 700, at least 750, at least 800, at least 850 or at least 900; a nMFI_{CD44} of at least 110, at least 120, at least 130, at least 140, at least 150, at least 200, at least 250, at least 300 or at least 350; or a nMFI_{CD105} of at most 180, at most 170, at most 160, at most 150, at most 140, at most 130, at most 120, at most 110 or at most 100;
a nMFI_{CD73} of at least 550, at least 600, at least 650, at least 700, at least 750, at least 800, at least 850 or at least 900; a nMFI_{CD44} of at least 110, at least 120, at least 130, at least 140, at least 150, at least 200, at least 250, at least 300 or at least 350; and a nMFI_{CD105} of at most 180, at most 170, at most 160, at most 150, at most 140, at most 130, at most 120, at most 110 or at most 100;
any one or more of a nMFI for CD73 of at least 700, a nMFI for CD44 of at least 200 or a nMFI for CD105 of at most 150; and/or
a nMFI for CD73 of at least 700, a nMFI for CD44 of at least 200 and a nMFI for CD105 of at most 150,
   preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

Present inventors found that the method for determining the osteogenic potential of the in vitro differentiated cells as taught herein may be used for selecting those MSC-donors which comprise MSCs which can be differentiated in vitro into cells having a clinically useful osteogenic potential.

Accordingly, a further aspect provides a method for selecting a subject for preparing in vitro differentiated cells of chrondro-osteogenic lineage, the method comprising:
recovering MSC from a biological sample of a subject;
obtaining in vitro differentiated cells from the MSC;
determining the osteogenic potential of the in vitro differentiated cells by a method as taught herein; and
selecting the subject for preparing in vitro differentiated cells of chondro-osteoblastic lineage if the in vitro differentiated cells have clinically useful osteogenic potential.

In particular embodiments, the recovering MSC from a biological sample of a subject may be performed as described elsewhere herein. In particular embodiments, obtaining in vitro differentiated cells from the MSC may be performed as described elsewhere herein.

In particular embodiments, the subject is a human subject.

The skilled person will understand that the definitions and particular embodiments as described herein are applicable to all methods and uses as disclosed herein.

The present application also provides aspects and embodiments as set forth in the following Statements:

Statement 1. Use of any one or more of CD73, CD105 or CD44 for determining osteogenic potential of in vitro differentiated cells.

Statement 2. A method for determining osteogenic potential of in vitro differentiated cells comprising measuring the quantity of the in vitro differentiated cells expressing any one or more of CD73, CD105, CD10 or CD44, and measuring the quantity of any one or more of CD73, CD105 or CD44 expressed by the in vitro differentiated cells.

Statement 3. The method according to statement 2, the method comprising:
(a1) measuring the fraction of the in vitro differentiated cells expressing any one or more of CD73, CD105, CD10 or CD44 on the cell surface of the in vitro differentiated cells;
(a2) measuring the quantity of any one or more of CD73, CD105 or CD44 on the cell surface of the in vitro differentiated cells;
(b1) comparing the fraction of the in vitro differentiated cells expressing any one or more of CD73, CD105, CD10 or CD44 as measured in (a1) with a cut-off value representing cells having a known osteogenic potential;
(b2) comparing the quantity of any one or more of CD73, CD105 or CD44 as measured in (a2) with one or more respective cut-off values representing cells having a known osteogenic potential;
(c1) finding a deviation or no deviation of the fraction of the in vitro differentiated cells expressing any one or more of CD73, CD105, CD10 or CD44 as measured in (a1) from said cut-off value;
(c2) finding a deviation or no deviation of the quantity of any one of CD73, CD105 or CD44 as measured in (a2) from said cut-off value; and
(d) attributing said deviation or no deviation to a particular determination of osteogenic potential of the in vitro differentiated cells.

Statement 4. The method according to statement 3, wherein said cut-off value of (b1) and said respective cut-off values of (b2) are cut-off values representing cells having clinically useful osteogenic potential.

Statement 5. The method according to statement 4, wherein:
a decreased fraction of the in vitro differentiated cells as measured in (a1) compared with the cut-off value of (b1) indicates that the in vitro differentiated cells have no clinically useful osteogenic potential, or
the same or an increased fraction of the in vitro differentiated cells as measured in (a1) compared with the cut-off value of (b1) indicates that the in vitro differentiated cells have clinically useful osteogenic potential; and
a decreased quantity of any one of CD73 and CD44 as measured in (a2) compared with the respective cut-off values of (b2), and/or an increased quantity of CD105 as measured in (a2) compared with the respective cut-off value of (b2) indicates that the in vitro differentiated cells have no clinically useful osteogenic potential, or
the same or an increased quantity of CD73 and CD44 as measured in (a2) compared with the respective cut-off values of (b2), and the same or a decreased quantity of CD105 as measured in (a2) compared with the respective cut-off value of (b2) indicates that the in vitro differentiated cells have clinically useful osteogenic potential.

Statement 6. The method according to any one of statements 3 to 5, wherein said cut-off value of (b1) is 90% of in vitro differentiated cells expressing any one or more of CD73, CD105, CD10 or CD44 on the cell surface; and wherein said cut-off value of (b2) is a normalized Median of Fluorescence Intensity (nMFI) for CD73 of 500, a nMFI for CD44 of 100 and/or a nMFI for CD105 of 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and/or the nMFI₁₀₅ is measured with an excitation wavelength of 633 nm and an emission wavelength of 660 nm for APC.

Statement 7. The method according to any one of statements 1 to 6, wherein the quantity of the in vitro differentiated cells expressing CD73, CD105, CD10 and CD44 is measured, and the quantity of CD73, CD105 and CD44 expressed by the in vitro differentiated cells is measured.

Statement 8. The method according to statement 7, wherein said cut-off value of (b1) is 90% of in vitro differentiated cells expressing CD73, CD105, CD10 and CD44 on the cell surface; and wherein said cut-off value of (b2) is a nMFI for CD73 of 500, a nMFI for CD44 of 100 and a nMFI for CD105 of 150, preferably wherein the nMFI_{CD73} is measured with an excitation wavelength of 633 nm and an emission wavelength of 660 nm for APC, the nMFI_{CD44} is measured with an excitation wavelength of 488 nm and an emission wavelength of 580 nm for PE, and the nMFI₁₀₅ is measured with an excitation wavelength of 633 nm and an emission wavelength of 660 nm for APC.

Statement 9. The method according to any one of statements 1 to 8, wherein the in vitro differentiated cells are obtained from mesenchymal stem cells (MSC).

Statement 10. The method according to any one of statements 1 to 9, wherein the in vitro differentiated cells are human cells.

Statement 11. A method for selecting a subject for preparing in vitro differentiated cells of chrondro-osteoblastic lineage, the method comprising:
recovering MSC from a biological sample of a subject;
obtaining in vitro differentiated cells from the MSC;
determining the osteogenic potential of the in vitro differentiated cells by a method as defined in any one of statements 1 to 10; and
selecting the subject for preparing in vitro differentiated cells of chondro-osteoblastic lineage if the in vitro differentiated cells have clinically useful osteogenic potential.

Statement 12. The method according to statement 11, wherein the subject is a human subject.

Statement 13. Use of CD73, CD105, CD44, and CD10 for determining osteogenic potential of in vitro differentiated cells.

Statement 14. A method for determining osteogenic potential of in vitro differentiated cells comprising measuring the quantity of the in vitro differentiated cells expressing CD73, CD105, CD10 and CD44, and measuring the quantity of any one or more of CD73, CD105 or CD44 expressed by the in vitro differentiated cells.

Statement 15. The method according to statement 14, the method comprising:
(a1) measuring the fraction of the in vitro differentiated cells expressing CD73, CD105, CD10 and CD44 on the cell surface of the in vitro differentiated cells;
(a2) measuring the quantity of any one or more of CD73, CD105 or CD44 on the cell surface of the in vitro differentiated cells;
(b1) comparing the fraction of the in vitro differentiated cells expressing CD73, CD105, CD10 and CD44 as measured in (a1) with a cut-off value representing cells having a known osteogenic potential;
(b2) comparing the quantity of any one or more of CD73, CD105 or CD44 as measured in (a2) with one or more respective cut-off values representing cells having a known osteogenic potential;
(c1) finding a deviation or no deviation of the fraction of the in vitro differentiated cells expressing CD73, CD105, CD10 and CD44 as measured in (a1) from said cut-off value;
(c2) finding a deviation or no deviation of the quantity of any one of CD73, CD105 or CD44 as measured in (a2) from said cut-off value; and
(d) attributing said deviation or no deviation to a particular determination of osteogenic potential of the in vitro differentiated cells.

Statement 16. The method according to statement 15, wherein said cut-off value of (b1) and said respective cut-off values of (b2) are cut-off values representing cells having clinically useful osteogenic potential.

Statement 17. The method according to any one of statements 14 to 16, wherein the quantity of any one or more of CD73, CD105, CD44 or CD10 on the cell surface of the in vitro differentiated cells is measured.

Statement 18. The method according to statement 16 or 17, wherein:
the same or an increased fraction of the in vitro differentiated cells as measured in (a1) compared with the cut-off value of (b1) indicates that the in vitro differentiated cells have clinically useful osteogenic potential; and
the same or an increased quantity of CD73, CD44 and/or CD10 as measured in (a2) compared with the respective cut-off values of (b2), and the same or a decreased quantity of CD105 as measured in (a2) compared with the respective cut-off value of (b2) indicates that the in vitro differentiated cells have clinically useful osteogenic potential.

Statement 19. The method according to statements 15 to 18, wherein said cut-off value of (b1) is 90% of in vitro differentiated cells expressing CD73, CD105, CD10 and CD44 on the cell surface; and wherein said cut-off value of (b2) is a normalized Median of Fluorescence Intensity (nMFI) for CD73 of 500, a nMFI for CD44 of 100, a nMFI for CD105 of 150 and/or a nMFI for CD10 of 40.

Statement 20. The method according to statements 15 to 18, wherein said cut-off value of (b1) is 90% of in vitro differentiated cells expressing CD73, CD105, CD10 and CD44 on the cell surface; and wherein said cut-off value of (b2) is a normalized Median of Fluorescence Intensity (nMFI) for CD73 of 500, a nMFI for CD44 of 150, a nMFI for CD105 of 150 and/or a nMFI for CD10 of 40.

Statement 21. The method according to any one of statements 14 to 20, wherein the quantity of CD73, CD105 and CD44 expressed by the in vitro differentiated cells is measured.

Statement 22. The method according to statement 21, wherein said cut-off value of (b2) is a nMFI for CD73 of 500, a nMFI for CD44 of 100 and a nMFI for CD105 of 150.

Statement 23. The use according to statement 13 or the method according to any one of statements 14 to 22, wherein the in vitro differentiated cells are obtained from mesenchymal stem cells (MSC).

Statement 24. The use according to statement 13 or the method according to any one of statements 14 to 23, wherein the in vitro differentiated cells are human cells.

Statement 25. A method for selecting a subject for preparing in vitro differentiated cells of chrondro-osteoblastic lineage, the method comprising:
recovering MSC from a biological sample of a subject;
obtaining in vitro differentiated cells from the MSC;
determining the osteogenic potential of the in vitro differentiated cells by a method as defined in any one of statements 14 to 24; and
selecting the subject for preparing in vitro differentiated cells of chondro-osteoblastic lineage if the in vitro differentiated cells have clinically useful osteogenic potential.

Statement 26. The method according to statement 25, wherein the subject is a human subject.

Statement 27. A method for determining osteogenic potential of in vitro differentiated cells, the method comprising, consisting essentially of or consisting of:
measuring the quantity of CD10 on the cell surface of the in vitro differentiated cells; and
determining that the in vitro differentiated cells have osteogenic potential if the in vitro differentiated cells have a nMFI for CD10 of at least 40, preferably the nMFI_{CD10} is measured with an excitation wavelength of 488 nm and an emission wavelength of 580 nm for PE.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1: Method for Obtaining Mesenchymal Stem Cells (MSC) and MSC-Derived Cells

### Mesenchymal Stem Cells

Undifferentiated MSC were prepared by obtaining human bone marrow (BM) aspirates from the iliac crest of healthy volunteer donors. After harvesting, bone marrow white blood cells were counted, seeded at a density of 50,000 cells/cm² in the culture medium, and incubated at 37° C. in a humidified incubator containing 5% CO₂. After 24 h, the culture medium was removed and cells fresh culture medium was added. The culture medium was replaced every 2-3 days. When over half of the colonies reached a confluence of 80% or when some colonies reached a confluence of 100%, cells were harvested (passage 1: P1). At this first passage, cells were directly cryopreserved in CryoStor® CS10 (BioLife Solutions Inc). To finish the culture process, MSCs were thawed, plated at 572 cells/cm² for second culture, and cultivated. Cells were harvested when over half of the colonies reached a confluence of 80% or when some colonies reached a confluence of 100% to obtain MSCs at passage 2 (P2). This cell product is referred to herein as “MSC”.

### Cell Product A (Also Referred to Herein as Bone Forming Cells A)

Conventional culture medium comprising 5% Octaserum (50:50 autologous serum and OctaPlasLG® (Octapharma)), FGF-b (CellGenix), TGFβ-1 (Humanzyme).

Freezing medium comprising 10% Octaserum (50:50 autologous serum and OctaPlasLG® (Octapharma)), 10% DMSO

The in vitro differentiated MSC-derived cells referred to herein as “Cell product A” were prepared by obtaining human BM aspirates from the iliac crest of healthy volunteer donors. After harvesting, bone marrow white blood cells were counted, seeded at a density of 50,000 cells/cm² in the culture medium, and incubated at 37° C. in a humidified incubator containing 5% CO₂. 4 days after cell seeding, non-adherent cells were removed and the medium was renewed with culture medium. 7 days and 11 days after seeding, half of the culture medium was removed and replaced with fresh one. Cells were cultured during primary culture for 14 days. At day 14, cells were harvested by detachment with Trypzean (Lonza) and by swirling and pipetting up and down (passage 1: P1). The intermediate cells were cryopreserved in CryoStor® CS10 (BioLife Solutions Inc.) or freezing medium and stored in liquid nitrogen.

For secondary culture, cells were thawed and re-plated at a density of 1144 cells/cm². Cells were cultured during secondary culture for 14 days. At day 28, cells were harvested by detachment with Trypzean (Lonza) and by swirling and pipetting up and down (passage 2: P2). To obtain the final cell product, cells were resuspended in OctaPlasLG® at a final concentration of 25×10⁶ cells/ml. This cell product is referred to herein as “Cell product A”.

### Cell Product B (Also Referred to Herein as Bone Forming Cells B)

Conventional culture medium comprising 5% OctaPlasLG® (Octapharma), 0.1 UI/ml heparin (LEO Pharma), FGF-b (CellGenix), TGFβ-1 (Humanzyme).

In vitro differentiated MSC-derived cells were prepared by obtaining human BM aspirates from the iliac crest of healthy volunteer donors. After harvesting, bone marrow white blood cells were counted, seeded at a density of 50,000 cells/cm² in the culture medium, and incubated at 37° C. in a humidified incubator containing 5% CO₂. 4 days after cell seeding, non-adherent cells were removed and the medium was renewed with culture medium. 7 days and 11 days after seeding, half of the culture medium was removed and replaced with fresh one to renew growth factors. Cells were cultured during primary culture for 14 days. At day 14, cells were harvested by detachment with Trypzean (Lonza) and by swirling and pipetting up and down (passage 1: P1). The intermediate cells were cryopreserved in CryoStor® CS10 (BioLife Solutions) and stored in liquid nitrogen.

Next, intermediate cells were thawed and re-plated for secondary culture at a density of 286 cells/cm². Cells were cultured during secondary culture for 14 days. At day 28, cells were harvested by detachment with Trypzean (Lonza) and by swirling and pipetting up and down (passage 2: P2). To obtain the final cell product, cells were resuspended in OctaPlasLG® at a final concentration of 25×10⁶ cells/ml. This cell product is referred to herein as “Cell product B”.

### Cell Product C (i.e., Cell Product C Fresh and Cell Products C Cryo; Also Referred to Herein as Bone-Forming Cells C)

Conventional culture medium comprising 5% OctaPlasLG® (Octapharma), 0.1 UI/ml heparin (LEO Pharma), FGF-b (CellGenix) and TGFβ-1 (Humanzyme).

20 to 60 ml of human bone marrow (BM) aspirates was obtained from the iliac crest of a healthy volunteer donor. After harvesting, bone marrow white blood cells were counted, seeded at a density of 50,000 cells/cm² in the culture medium, and incubated at 37° C. in a humidified incubator containing 5% CO₂. 4 days after cell seeding, non-adherent cells were removed and the medium was renewed with culture medium. 7 days and 11 days after seeding, half of the culture medium was removed and replaced with fresh one to renew growth factors. Cells were cultured during primary culture for 14 days. At day 14, cells were harvested by detachment with Trypzean (Lonza) and by swirling and pipetting up and down (passage 1: P1). The intermediate cells were cryopreserved (in CryoStor® CS10) and stored in liquid nitrogen. Each cell stock was issued from one donor, with no pooling between donors.

Next, intermediate cells were thawed and re-plated for secondary culture at a density of 572 cells/cm². Cells were cultured during secondary culture for 10 days. At day 24, cells were harvested by detachment with Trypzean (Lonza) and by swirling and pipetting up and down (passage 2: P2). The intermediate cells were cryopreserved (in CryoStor® CS10) and stored in liquid nitrogen.

Subsequently, intermediate cells were thawed and re-plated for tertiary culture at a density of 572 cells/cm². Cells were cultured during tertiary culture for 10 day. At day 34, cells were harvested by detachment with Trypzean (Lonza) and by swirling and pipetting up and down (passage 3: P3). To obtain the final cell product, cells were resuspended in OctaPlasLG® at a final concentration of 25×10⁶ cells/ml. This cell product will be referred to herein as “Cell product C—fresh”.

At the end of the tertiary culture, cells were also cryopreserved for long-time storage. Thereto, the cells were resuspended in cryopreservation medium to reach the desired concentration (25×10⁶ cells/ml). The cell suspension was then transferred into cryotubes which were stored in liquid nitrogen. This cell product will be referred to herein as “Cell product C—cryo” or “bone-forming cells C cryo(preserved)” also abbreviated as “B-F cells C”. The cryopreservation medium was:
CryoStor® CS10 (BioLife Solutions), or
50% (v/v) CryoStor® CS10 (BioLife Solutions) and 50% (v/v) human serum albumin (Octapharma), or
95% (v/v) CryoStor® CS10 (BioLife Solutions) and 5% (v/v) human serum albumin (Octapharma), or
80% (v/v) Hypothermosol® (BioLife Solutions), 10% (v/v) DMSO, and 10% (v/v) human serum albumin (Octapharma)

### Example 2: In Vivo Bone Forming Properties of MSC-Derived Cells of Chondro-Osteoblastic Lineage

### Materials and Methods

### Cell Culture

MSC, bone-forming cells A, B and C were prepared as described in Example 1.

### Mice

Female NMRI-Nude (nu/nu) mice of 9-10 weeks were purchased from Janvier S.A.S. (Le Genest-St-Isle, France) and housed in standard conditions with food and water ad libitum. 196 mice were used in total for the present study.

### Calvaria Bone Formation Mouse Model

Twelve-week-old female NMRI-Nude (nu/nu) mice (n=137) were anesthetized with isoflurane (IsoFlo®) and received a single subcutaneous administration of MSC, bone-forming cells A (generated with FGF-2 and TGFβ1), or bone-forming cells B (generated with FGF-2, TGFβ1 and heparin) (2.5×10⁶ cells in 100 μl per mouse) or excipient (100 μl) over the calvaria bone. To label bone neo-formation over time, calcium-binding fluorochromes were sequentially administered to mice. Alizarin red (red), calceins (green and blue) and tetracycline (yellow) (all from Sigma-Aldrich®) were injected intraperitoneally 3 days before and 4, 8, and 12 days after cell administration, respectively. Experimental animals were monitored for body weight, general clinical signs, and clinical signs at site of administration for 2 weeks following the administration. Mice were euthanized 2 weeks after cell administration by cervical dislocation and the calvaria of each mouse was harvested to assess bone formation properties of bone-forming cells by X-ray imaging, histomorphometry (quantification of bone formation) and immunofluorescence.

### Calvaria Bone Formation Mouse Model—Cell Product C Cryo

Twelve-week-old female NMRI-Nude (nu/nu) mice were anesthetized with isoflurane (IsoFlo®) and received a single subcutaneous administration of cell product C cryo (2.5×10⁶ cells in 100 μl per mouse) or excipient (100 μl) over the calvaria bone. To label bone neo-formation over time, calcium-binding fluorochromes were sequentially administered to mice. Alizarin red (red), calceins (green and blue) and tetracycline (yellow) (all from Sigma-Aldrich®) were injected intraperitoneally 2 or 3 days before and 5, 12, and 19 days after cell administration, respectively. Experimental animals were monitored for body weight, general clinical signs, and clinical signs at site of administration for 4 weeks following the administration. Mice were euthanized 4 weeks after cell administration by cervical dislocation and the calvaria of each mouse was harvested to assess bone formation properties of bone-forming cells by X-ray imaging, histomorphometry (quantification of bone formation) and immunofluorescence.

### Sample Embedding and Histological Sectioning

For histomorphometry, ALP, TRAP (tartrate-resistant acid phosphatase), Masson Trichrome Goldner stainings and immunofluorescence, calvarias were fixed and dehydrated with successive incubations in 70%, 80% and 90% ethanol bath, for 12 hours each, at 4° C. with gentle shaking, and embedded in hydroxyethylmethacrylate (HEMA) plastic resin (HistoResin, Leica®). Four μm-thick and 8 μm-thick coronal sections were cut using a microtome (Leica®, RM2255). For safranin-orange staining and immunoperoxidase, calvarias were fixed in 3.7% formaldehyde for 24 hours, decalcified in 10% ethylenediaminetetraacetic acid (EDTA) pH 7.4 for three days and embedded in paraffin. Seven μm-thick coronal and sagittal paraffin sections were cut using a microtome (Leica®, RM2255).

### Immunofluorescence Staining

Assessment of the human and murine collagen I by immunofluorescence was performed on 4 μm-thick coronal plastic histological sections of calvaria. Briefly, after a step of permeabilization using a solution of PBS 1×/Triton 0.3% for 30 min at room temperature (RT), the histological sections were incubated for 1 hour at RT in the blocking solution (i.e., PBS/BSA/horse serum/Triton™) to sature non-specific binding sites. The histological slides were then incubated overnight at 4° C. with mouse anti-human and rabbit anti-murine collagen I primary antibodies (Abeam; #ab138492 and Abeam; #ab21286 respectively). After 3 steps of rinsing in PBS for 5 min at RT, blocking was realized with the blocking solution for 1 hour at RT. The secondary antibodies diluted in the blocking solution was then added for 2 hours at RT protected from the light. The secondary antibody Alexa Fluor® 488 Donkey anti-rabbit IgG H&L (ThermoFisher, #A21206) and Alexa Fluor® Cy3® Goat anti-mouse IgG H&L (Abeam; #ab97035) were used to visualize the murine collagen I in green and the human collagen I in red, respectively. The slides were then rinsed 3 times in PBS 1× for 5 min at RT and incubated with NucBlue® solution for 1 min at RT to stain the nucleus. Finally, the slides were briefly rinsed once in PBS then mounted in GlycerGel® reagent. As negative control of immunofluorescence, omission of primary antibody was performed on adjacent histological slide.

### Histological Staining

Osteoblastic and osteoclastic activities were assessed on calvaria sections respectively using ALP and TRAP enzymatic activity detection methods respectively. For ALP staining, 4 μm-thick calvaria coronal plastic sections were incubated for 1 hour with a solution of Fast Blue RR Salt (Sigma-Aldrich®) and Naphtol AS-MX phosphate alkaline (Sigma-Aldrich®). TRAP staining was performed on 8 μm-thick calvaria coronal plastic sections using the Acid Phosphatase, Leukocyte (TRAP) Kit, (Sigma-Aldrich®) according to manufacturer's instructions. To assess the status of mineralization of the neo-formed bone, Masson Trichrome Goldner staining was performed on the calvaria sections stained with ALP using a kit (Bio-Optica®) according to manufacturer's instructions. To evidence cartilage formation, safranin-orange staining was performed on 7 μm-thick calvaria sagittal paraffin sections. Briefly, after deparaffinization, histological sections were successively incubated in Weigert's Hematoxylin (Klinipath®) for 10 min, 0.1% Fast Green (Klinipath®) for 5 min, 1% acetic acid (VWR Chemicals) for 15 sec and 0.1% safranin-orange (Fluka® ref: 84120) for 5 min. After dehydration, slides were mounted with glass coverslips using Pertex® (HistoLab®). Digital images were taken with an optical microscope (Leica®) and the Leica® LAS EZ software.

### Immunoperoxidase

After deparaffinization, 7 μm-thick calvaria coronal or sagittal paraffin sections were successively incubated with 2.5% hyaluronidase (Sigma-Aldrich®) for 30 min at 37° C., in 3% H₂O₂ (Sigma-Aldrich®) for 30 min at room temperature, in PBS containing 0.3% Triton X-100 (Sigma-Aldrich®) for 30 min at room temperature, and in blocking solution (i.e., PBS/BSA/horse serum/Triton) for 1 hour at RT at room temperature. Sections were incubated overnight at 4° C. with mouse anti-human type I collagen primary antibodies (Abeam, ab90395), rabbit anti-murine type I collagen primary antibodies (Abeam, ab21286) or with rabbit anti-Ku80 primary antibodies (Abeam, ab80592). Staining was visualized using a Vectastain kit (Vector Laboratories, PK6200) and 3,3′ diaminobenzidine (Vector Laboratories), according to manufacturer's instructions. Sections were counterstained with Mayer's Hematoxylin (Klinipath®). Slides were mounted with glass coverslips using Pertex®.

### Quantification of Bone Formation by X-Ray Analysis (Bone-Forming Cells C)

At euthanasia, ex vivo X-ray imaging of the calvaria of each mouse placed side by side was performed using the Faxitron® MX-20 device. Digital images were taken at a 1.5× magnification in manual mode with voltage set at 35 kV, exposure time at 4.8 sec, brightness/contrast at 8300/6000. The X-ray images generated are grey level images with grey intensity values ranging from 0 (black region) to 255 (white region) and are directly proportional to radio-opacity and therefore to bone opacity or bone thickness. The grey level intensity value of the osteoinduction part of the bone formation (mineralized nodules discarded from the selection) on parietal bones (manual selection) was analysed using histogram tool of AdobePhotoshop® software.

X-ray imaging and AdobePhotoshop® software were also used to quantify the surface of mineralized nodules (manual selection).

### Histomorphometrical Analyses of Calvarias: Quantification of Bone Formation

Quantification of bone formation (i.e., absolute bone formation) was performed on plastic embedded tissues. Measures of the absolute neo-formed bone thickness (from basal mineralization front fluorescently labelled by alizarin red to bone neo-formation fluorescently labelled by calcein and tetracycline) with and without mineralized nodules were measured (in μm) on 4 μm-thick coronal section by ZEN® image analysis software (Zeiss). For each animal, 4 measurements of absolute thicknesses were performed on 5 independent levels, with a distance of 200 μm between each level. As the first step, mean of thickness (with or without nodules)±SD (i.e., mean of the 4 measures per level on the 5 levels) were calculated for each animal.

### Quantification of the Surface Area of Neo-Formed Bone on Histological Images (ImageJ® Software)

For the surface area analysis of osteoinduction and osteogenic nodules, digital images of 6 independent levels taken every 2 levels after the coronal suture were taken from plastic resin histological sections (4 μm) of calvaria, using a combination of multiple fluorescence and brightfield filters of the fluorescent microscope (Zeiss Axioscope A1, Zeiss, Germany). On each measured level, the selection of the osteoinducted bone neo-formation was manually defined in brightfield stiches images using ImageJ® software. The mineralized and total surface areas of this selection were measured (in mm²). The same procedure was performed for the mineralized and total surface areas of the osteogenic nodules.

For the osteoinduction and the osteogenic nodules, the mean of the total surface area and the mean of the mineralized surface area was then calculated per experimental animal and per group. The total surface area of the bone neo-formation was finally calculated as the sum of the osteoinduction and osteogenic nodules surface areas.

### Statistical Analyses

Results were expressed as means±standard deviation (SD). Statistical analyses were performed using JMP® (SAS Institute Inc.) or GaphPad Prism® software. Differences between groups were considered statistically significant when p<0.05.

### Results

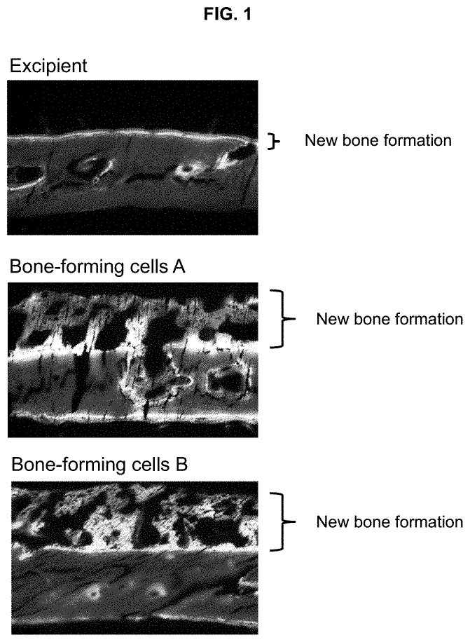

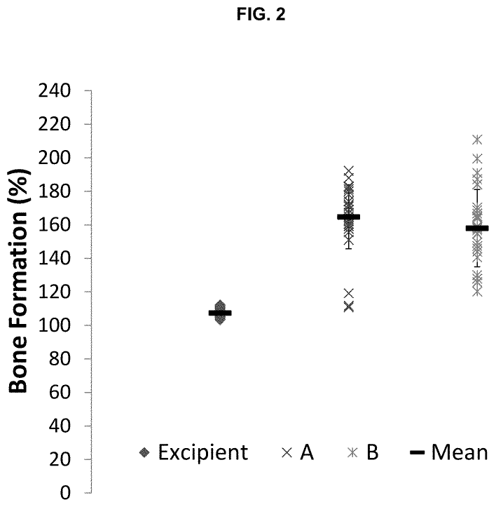

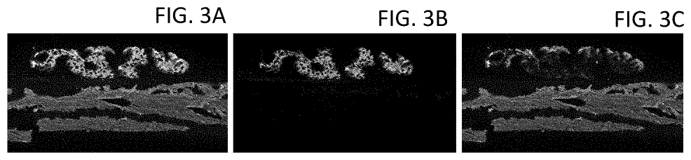

Both bone-forming cells A (generated with FGF-2 and TGFβ1) and bone-forming cells B (generated with FGF-2, TGFβ1 and heparin) showed significant higher bone formation than controls (excipient) 2 weeks after administration (FIGS. 1-2, Table 1). More particularly, FIG. 3 shows that bone-forming cells B displayed osteoinductive properties (homogenous bone formation from murine origin over the calvaria), and osteogenic properties (mineralized nodules from human and murine origins).

TABLE 1
Quantification of bone formation (%) on murine calvaria slices.
Murine calvaria have been treated with excipient (negative
control), bone-forming cells A or bone-forming cells B.

| | | | % of bone |
|---|---|---|---|
| | Nb. of | Nb. of | formation |
| | batches | animals | Mean ± SD |
| Excipient | — | 59 | 107 ± 2 |
| Bone-forming cells A | 10 | 39 | 165 ± 19 |
| Bone-forming cells B | 7 | 30 | 158 ± 23 |

Abbreviations: SD: standard deviation

Osteoinductive properties (i.e., quantity of murine bone newly formed post-implantation) were equivalent for bone-forming cells A and B (FIGS. 1-2).

Very interestingly, the bone-forming cells B of the present invention displayed potent osteogenic properties and osteoinductive properties as shown by the high quantity of human and murine bone newly formed post-implantation (human and murine ColI IF staining, FIG. 3).

The presence of nodules was observed in 7/8 donors and 80% of mice of bone-forming cells B and in 4/11 donors and 20% of mice for bone-forming cells A. No nodule was observed after MSC or excipient administration. In addition to osteoinduction activities, bone-forming cells B thus promote a high osteogenic activity highlighted by the presence of large mineralized nodules observed in 80% of treated mice while bone-forming cells A display weak osteogenic activity i.e., small nodules in only 20% of treated mice (Table 2).

TABLE 2
Quantification of the presence of mineralized nodules on murine
calvaria two weeks after administration over the calvaria of
excipient, MSC, bone-forming cells A or bone-forming cells B

| Osteogeny | | | | | | |
|---|---|---|---|---|---|---|
| occurrence | Donor | | Batch | | Animal | |
| Excipient | NA | | NA | | 0/32 | (0%) |
| MSC | 0/2 | | 0/2 | | 0/14 | (0%) |
| Bone-forming cells A | 4/10 | (40%) | 4/11 | (36%) | 9/45 | (20%) |
| Bone-forming cells B | 7/8 | (88%) | 7/8 | (88%) | 37/46 | (80%) |

Abbreviations: MSC: mesenchymal stem cells; NA: not applicable

The histology staining of murine bone calvaria coronal sections two weeks after administration (excipient only, MSC, bone-forming cells A (generated with FGF-2 and TGFβ1; b-f cells A) or bone-forming cells B (generated with FGF-2, TGFβ1 and heparin; b-f cells B)) revealed that all treated conditions (MSC, b-f cells A and b-f cells B) have a high osteoinduction potential with a medium remodeling activity (ALP and TRAP staining) in the bone formed by osteoinduction.

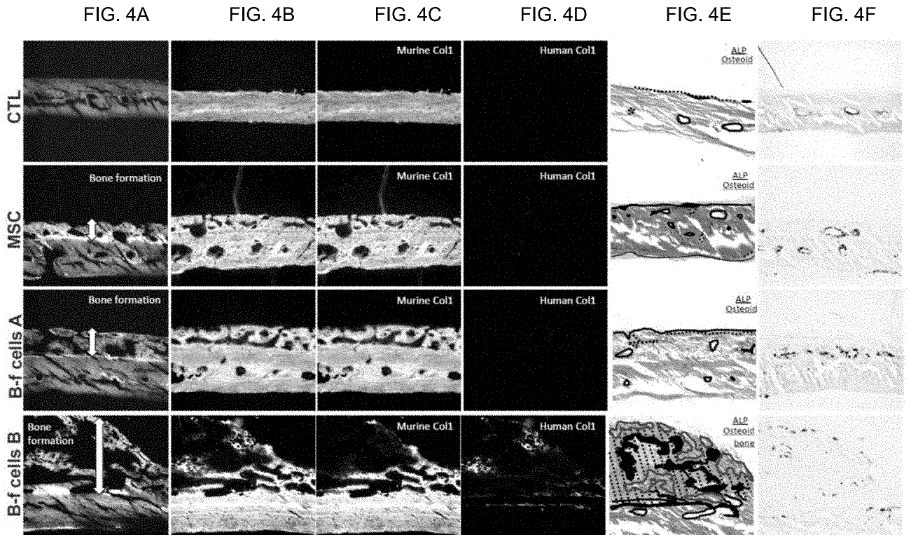

Interestingly, the mineralized nodules observed in mice treated with bone-forming cells B were constituted of both murine (host) and human (donor) bone tissues (evidenced by human and murine type I collagen staining) demonstrating that the nodules were formed by both bone formation processes: osteogeny (donor bone formation) and osteoinduction process (host bone formation). In addition to a high osteoblast and osteoclast activities (ALP+TRAP staining), the nodules exhibited osteoid tissue (non-mineralized tissue) suggesting that bone formation was still progressing two weeks after administration, while the osteoinduction process observed in all conditions was already completed (FIG. 4).

FIG. 4 shows that human bone formation (i.e., osteogeny) (observed with anti-human type I collagen staining), and high osteoblast and osteoclast activities (observed with ALP+Goldner staining and TRAP staining respectively) were detected mostly in nodules of mice administered with bone-forming cells B, thereby showing that the bone formation process in the nodules was ongoing and was not completed at 2 weeks, unlike the osteoinduction process of MSC and bone-forming cells A that seemed completed. All treated conditions (MSC, b-f cells A, b-f cells B) had a high osteoinduction potential with a moderate remodeling activity (ALP and TRAP staining) in the osteoinducted bone formation (FIG. 4).

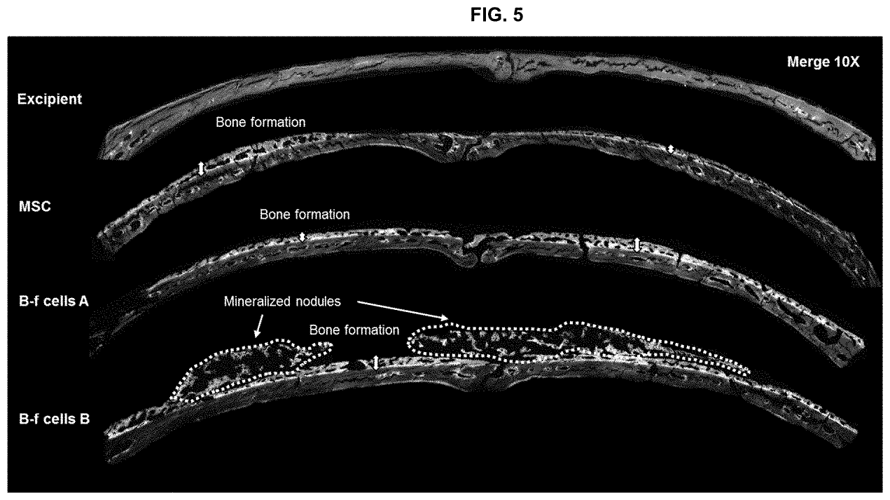

The bone neo-formation was assessed by fluorescence two weeks after treatment with excipient only, MSC, b-f cells A or b-f cells B (FIG. 5). To this end, at specific time points, bone calcium binding fluorescent dyes (i.e., alizarin red, calcein green and blue, tetracycline yellow) were administered to the mice to label the neo-formed bone. The last fluorochrome to be administrated was tetracycline, administrated 12 days after administration of the cells.

As shown in FIG. 5, the nodules of the mice administered with bone-forming cells B were mostly stained by tetracycline fluorochrome (yellow staining have been surrounded in dotted line in FIG. 5) confirming a later stage of formation compared to osteoinduction observed in the osteoinducted bone formation (alizarin red (red), calcein (green) and calcein blue (blue): these stainings appear in light grey and double arrows indicate the bone formation thickness).

The bone neo-formation of treated mice was assessed by quantification of the surface area of neo-formed bone on histological images (ImageJ® software). The total surface area of the neo-formed bone was determined by summing the osteo-induced and the bone nodule surface areas for each analyzed level and each mouse.

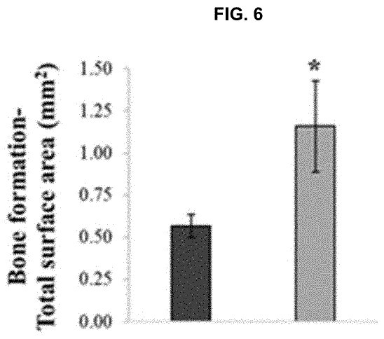

The results show that bone-forming cells B (n=7 mice, shown in FIG. 6 in light grey) significantly enhanced bone neo-formation 2 weeks after administration of the cells by about 2-fold compared to MSC (n=6 mice, shown in FIG. 6 in dark grey; Table 3). This difference was due to the high osteogeny property displayed by the bone-forming cells B and the absence of such property for MSC.

TABLE 3
Total bone neo-formation are measured on coronal sections
including the osteoinduction and the osteogenic formation

| | | | | | | Total | |
|---|---|---|---|---|---|---|---|
| | | Osteoinduction | | Osteogeny (nodules) | | (osteoinduction + osteogeny) | |
| Cell type | | Mineralized | Total area | Mineralized | Total area | Mineralized | Total area |
| (from the | Number of | area (mm²) | (mm²) | area (mm²) | (mm²) | area (mm²) | (mm²) |
| same donor) | animals | (mean ± SD) | (mean ± SD) | (mean ± SD) | (mean ± SD) | (mean ± SD) | (mean ± SD) |
| MSC | 6 | 0.42 ± 0.09 | 0.57 ± 0.17 | 0 | 0 | 0.42 ± 0.09 | 0.57 ± 0.17 |
| b-f cells B | 7 | 0.43 ± 0.16 | 0.59 ± 0.25 | 0.22 ± 0.19 | 0.57 ± 0.53 | 0.65 ± 0.30 | 1.16 ± 0.71 |

Abbreviations:
MSC: mesenchymal stem cells;
SD: standard deviation

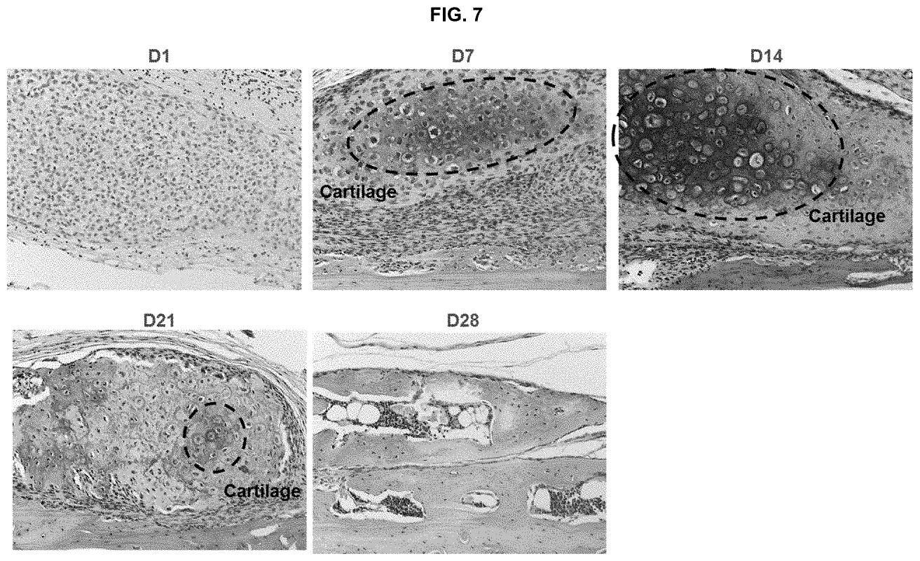

Furthermore, the evaluation of the bone neo-formation over time using histological staining revealed that nodules observed on the top the calvaria of mice administered with bone-forming cells B were ossifying via an endochondral ossification mechanism. In FIG. 7, the safranin-orange staining shows proteoglycan (specific to cartilage) matrix (area surrounded by dashed lines); nuclei; bone tissue; and cytoplasm. Contrary to the osteo-induced bone that was produced by intramembranous ossification, bone nodules were produced through endochondral ossification, with cartilage matrix occurring between 1 week and 3 weeks after administration (FIG. 7).

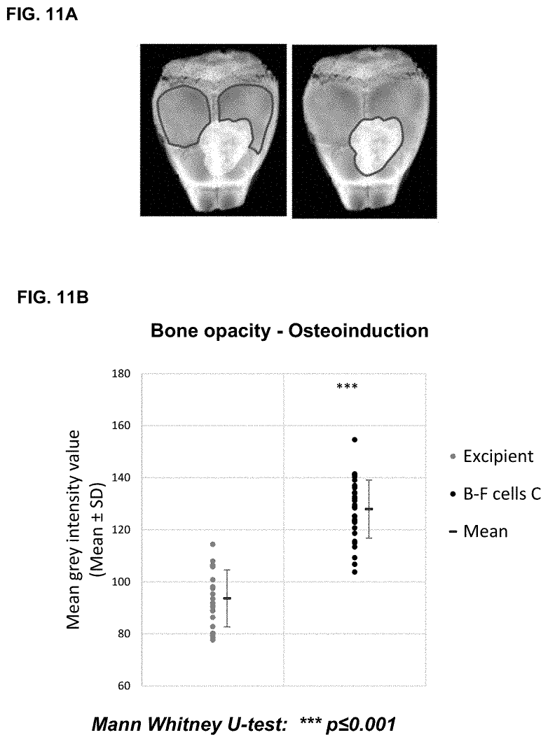

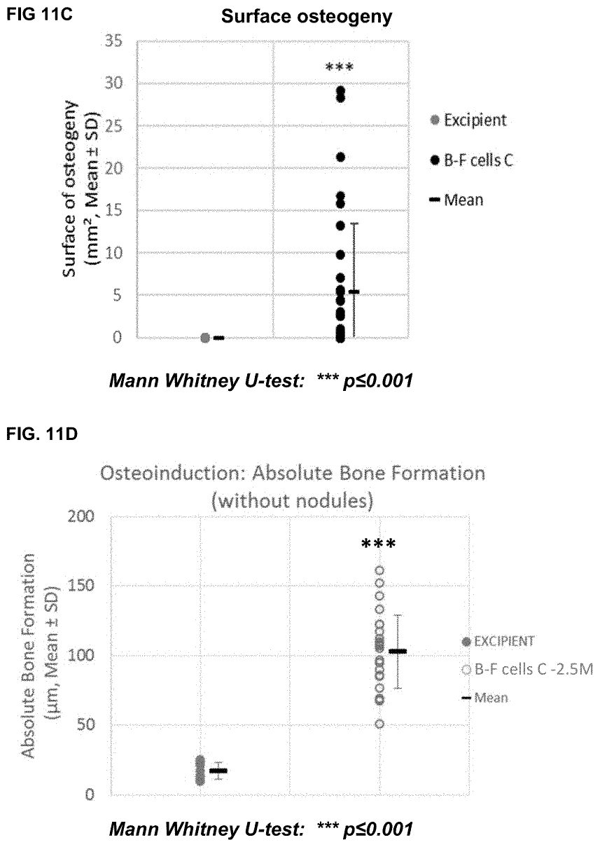

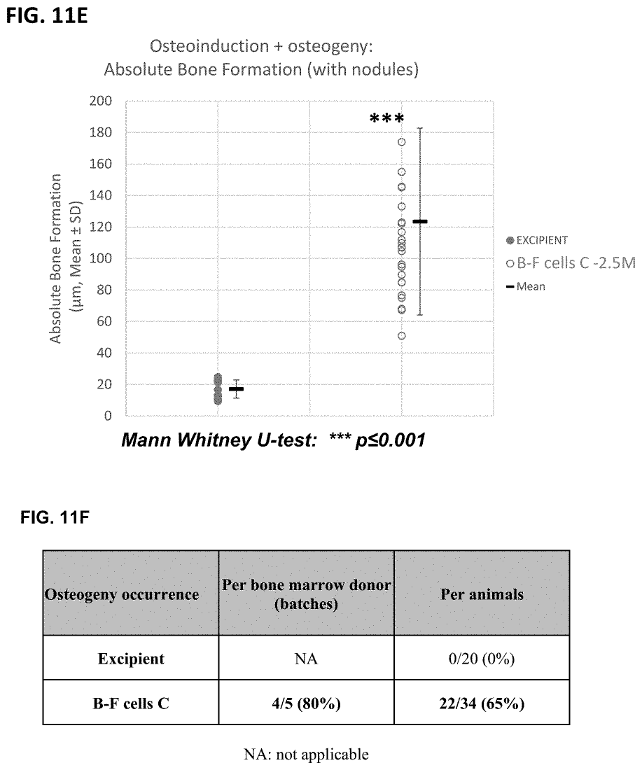

Immunohistochemistry stainings targeting human type I collagen, murine type I collagen and human nucleus (i.e. Ku80) performed 4 weeks after the administration of bone-forming cells B confirmed the presence of human bone in the nodules. Moreover, Ku80 staining revealed that bone-forming cells B were engrafted in the bone matrix (nodules) and became osteocytes after in vivo administration. Mice which have been administered cell product C cryo showed higher bone formation than controls 4 weeks after administration (FIG. 11A-FIG. 11C). The bone opacity was significantly higher for bone-forming cells C cryo compared to excipient (FIG. 11B). The surface of osteogeny was significantly higher compared to excipient in which no mineralized nodules were observed (FIG. 11C). Histomorphometric measures of the osteoinduction with or without osteogeny (represented by the absolute bone formation) was significantly higher for bone-forming cells C cryo compared to excipient (FIG. 11D and FIG. 11E). Also, in addition to osteoinduction activities, bone-forming cells C cryo promoted a high osteogenic activity highlighted by the presence of mineralized nodules. This osteogenic activity was observed in 4/5 bone marrow donors (or batch production) and 65% of mice (FIG. 11F). One donor/batch was considered to be osteogenic (positive) when at least one mineralized nodule was observed in one mouse per group. No nodule was observed after excipient administration.

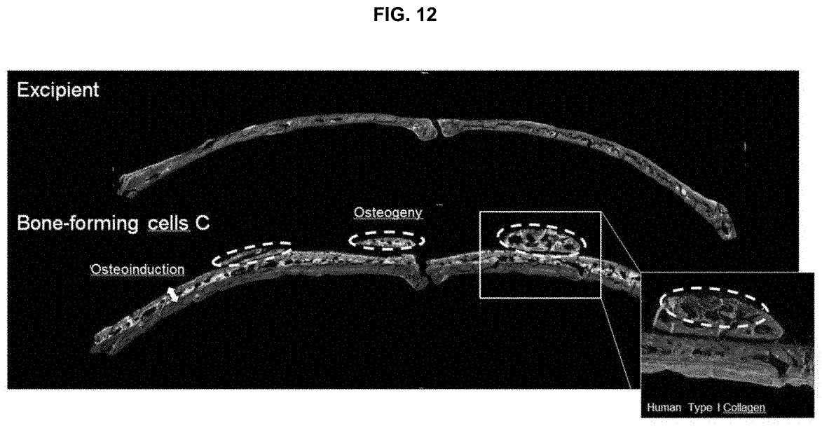

More particularly, cell products C cryo displayed both osteoinductive properties (homogenous bone formation from murine origin over the calvaria) and osteogenic properties (mineralized nodules from human and murine origin) (FIG. 12).

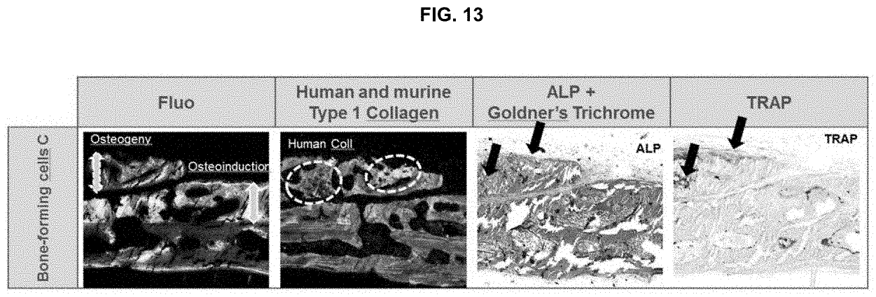

Intramembranous host ossification was induced along the calvarial surface (FIG. 12 and FIG. 13). More particularly, bone-forming cells C cryo displayed osteoinduction and osteogenic properties (FIG. 13, “fluo”). Mouse/human type I collagen double-immunolabeling (FIG. 13, “human type I collagen”) revealed the presence of bone of host and donor origins (osteogeny). Osteoblast (FIG. 13, “ALP+”) and osteoclast (FIG. 13, “TRAP”) activities were mostly detected in mineralized nodules showing that the bone remodeling process in the nodules was still ongoing 4 weeks post-administration. This observation was dependent on the size of the nodule: the larger the nodule, the more ALP and TRAP activities are still present at 4 weeks post-administration. Weak osteoid (FIG. 13, “Goldner's Masson trichrome staining”) was highlighted indicating that the bone formation process is completed.

Accordingly, bone-forming cells C cryopreserved increased bone neo-formation.

This demonstrates the usefulness of cell products and cell composition as described in the specification and the examples for treatment of bone defects in flat bones as well as long bones.

### Example 3: In Vivo Mouse Femoral Segmental Sub-Critical Size Defect (Sub-CSD) Repaired by Bone-Forming Cells A, Bone-Forming Cells B and Bone-Forming Cells C Cryopreserved

### Experimental Procedures

### Cell Culture

Bone-forming cells A, bone-forming cells B and bone-forming cells C cryopreserved were prepared as described in Example 1.

### Femoral Segmental Sub-CSD Model

The surgical procedure was performed under aseptic conditions according to literature (Manassero et al., 2013, Tissue Engineering, Part C Methods, 19(4):271-80; Manassero et al., 2016, Journal of Visualized Experiments; (116): 52940). Briefly, 13-week-old female NMRI-Nude (nu/nu) mice (n=73) were anaesthetized with an intraperitoneal injection of a mix of dexmedetomidine hydrochloride (Dexdomitor®, Orion Pharma, 1 mg/kg of body weight) and ketamine (Nimatek®, Euronet, 150 mg/kg of body weight) and were placed in a ventral position on a warming plate. After applying a 6-hole titanium micro-locking plate (RISystem AG®) fixed with 4 or 5 screws on the anterior side of the left femur, a 2-mm long mid-diaphyseal femoral osteotomy was performed using a Gigli saw and a jig (RISystem AG®). As preventive medication, antibiotics (Baytril®, 10 mg/kg of body weight) were administered the day before the surgery (in drinking water) and analgesic (buprenorphine hydrochloride, Temgesic®, Schering-Plough, 0.1 mg/kg of body weight) was administered the day before the surgery and every 12 hours for at least 3 days following the surgery. MSC-derived cells (2.25×10⁶ cells in a volume of 30 μl per mouse) or the excipient (control group) was administered on the day of the surgery (just after closing the wound with surgical sutures), locally at the site of the bone defect, by percutaneous injection using a 50 μl-Hamilton syringe. Mice were euthanized 6 or 10 weeks after cell or excipient administration by cervical dislocation. The left femur of each mouse was dissected, harvested and kept in 0.9% NaCl at room temperature until X-Ray imaging.

### Quantification of Bone Repair by X-Ray Analyses

In vivo X-ray imaging of the left femur of each mouse was performed, using the Faxitron® MX-20 device just after the surgery to control the plate fixation, the segmental femoral defect size and to get a baseline, and every two weeks. Digital images were taken in mediolateral and anteroposterior views at a 5× magnification in manual mode with voltage set at 35 kV, exposure time at 4.8 sec, brightness at 4,300 and contrast at 7,100. Ex vivo X-ray imaging was performed on left femurs harvested at euthanasia, 6 weeks after cell administration. For bone-forming cells A and bone-forming cells B experiment, the defect size was quantified for each mouse over time by measuring the distance (μm) between the two edges of the bone defect at three locations (right, middle and left of the defect) on mediolateral and anteroposterior X-ray images (total of 6 measures), using ImageJ® software. The mean of the six measurements was calculated for each mouse at each time point.

For bone-forming cells C cryopreservated experiment, the defect size was quantified for each mouse over time by measuring the distance (μm) between the two edges of the bone defect at two locations (both cortices) on mediolateral and anteroposterior X-ray images (total of 4 measures), using ImageJ® software. The mean of the four measurements was calculated for each mouse at each time point.

The RUS (radiographic union score) adapted for the SFCSD model is a semi-quantitative measurement based on the presence or absence of a bone neo-formation, a bridging and a fracture line (from anteroposterior and medio-lateral radiographic images). The scoring corresponds to the sum of 4 scores determined at 2 cortical defect sites on both views (total of 4 scores ranging from 1 to 4 each). The scoring ranges therefore from 4 (no sign of healing) to 16 (complete fusion).

Fusion score is a binary score which assess the fusion rate between the edges of the femoral defect. The radiological criteria utilized to define fusion is the visualization of bridging of the defect in at least 3 cortices (Cekiç E et al., Acta Orthop Traumatol Turc. 2014, 48(5), 533-40). The score is 0 (no fusion) or 1 (fusion). For this parameter, only the last time point was analysed (W10, here).

### Micro-Computed Tomography (Micro-CT) Analyses

After harvesting at euthanasia, the left femurs were fixed with 3.7% formaldehyde and transferred to the Center For Microscopy and Molecular Imaging (CMMI, ULB, Gosselies, Belgium) for micro-CT analyses. Samples were scanned using a multimodal microPET/CT nanoScan® PET/CT camera (Mediso) and the Nucline™ v2.01 software (Mediso). Scans were made using a semi-circular scan, the maximum zoom, a tube tension of 35 kVp, 720 projections per gantry rotation, an exposure time of 300 ms per projection, a detector pixel binning of 1 to 1. The scan lengths in the X and Y dimensions were adapted for each acquisition. The total duration of micro-CT scanning was 3 min 42 sec. Each micro-CT scan was post-reconstructed with a cubic voxel of 40 μm-side using a Shepp-Logan filter and a multi-sampling mode of 8 regular samples. The dimensions of the X and Y images were adapted for each reconstruction. The size of the Z-images corresponded to the scan length defined for the acquisition. A qualitative evaluation of bone repair was performed on the micro-CT images after reorienting the bone with the Z-axis (scanner axis) and cropping the image from one proximal to the other proximal screw in the femoral bone on the Z-axis, and as narrow as possible in the transverse (X-Y) plane. Then, a 3D Maximum Intensity projection (MIP) rendering was produced. To quantitatively assess bone repair, a virtual cylinder of 2 mm-diameter and 2 mm-axial length was placed in the defect space on the micro-CT scans and the mean bone volume was evaluated in this cylinder by thresholding voxels with radiological intensity equal or higher than 1500 HU.

### Results

### Bone-Forming Cells B and Bone-Forming Cells C Cryo Improved the Repair of Mice Femoral Sub-Critical Segmental Defect

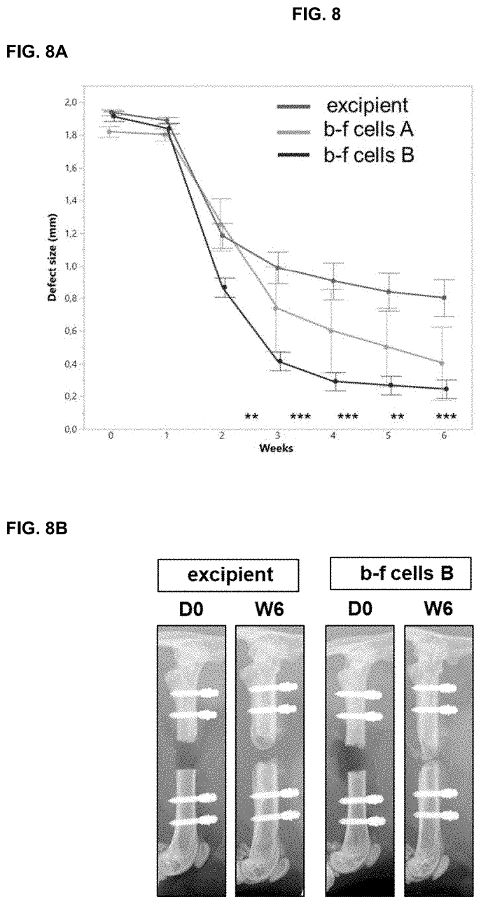

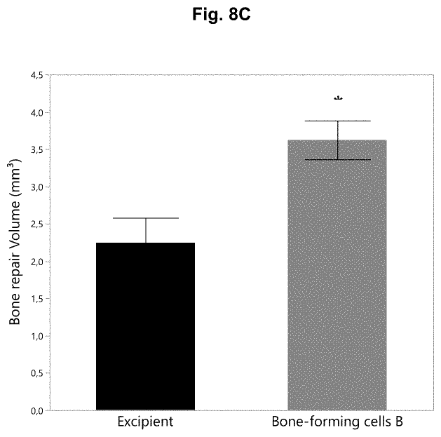

In the sub-critical size segmental defect (CSD) model in NMRI-Nude mice, bone-forming cells B (n=12 mice, 2 batches) improved fracture repair as shown by a significant reduction of the bone defect size compared to the excipient (n=11 mice), and to the bone-forming cells A (n=4 mice) from 2 to 6 weeks after administration (FIG. 8A).

X-ray images of segmental femoral defects at D0 and 6 W after administration of the excipient, bone-forming cells A (not shown) or bone-forming cells B showed a reduction of the bone defect size in mice administered with bone-forming cells B according to an embodiment of the invention compared to mice administered with the excipient (FIG. 8B) or bone-forming cells A (not shown).

The bone repair volumes of segmental femoral defect were quantified by micro-computed tomography (micro-CT) analyses at 6 W after administration of the excipient and bone-forming cells B. The results confirmed that bone-forming cells B induced higher bone repair compared to excipient (FIG. 8C).

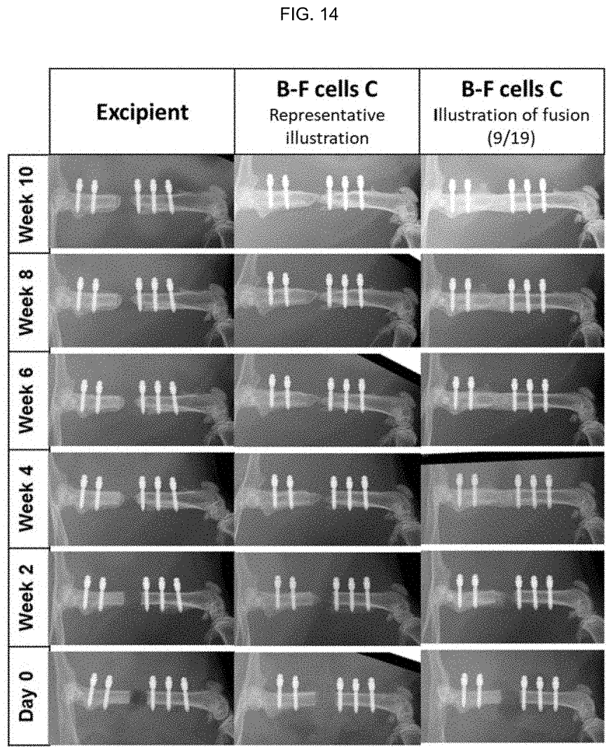

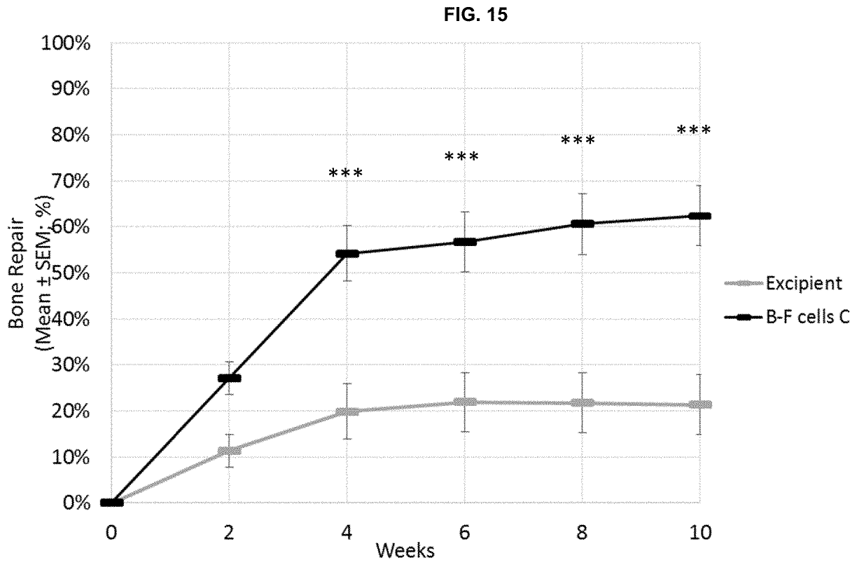

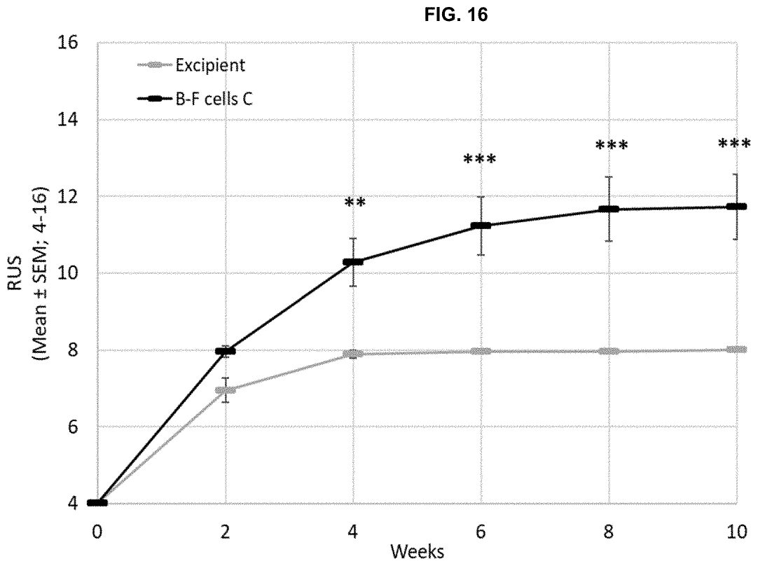

Bone-forming cells C cryopreserved significantly improved and accelerated the percentage of bone fracture repair in the segmental femoral sub-CSD model, (FIG. 14 and FIG. 15) compared to the excipient from 2 to 10 weeks after administration (n=38 mice (19 treated and 19 control with excipient, 2 batches; p<0.001). Moreover, the RUS score was significantly increased for bone-forming cells C cryopreserved group compared to excipient group (FIG. 16). And finally, the fusion rate was also improved with fusion for 9/19 (47%) of mice 10 weeks after administration of bone-forming cells C cryopreserved compared to no fusion after excipient administration.

### Example 4: In Vitro Cell Characterisation of MSC-Derived Cells Showing Osteogenic Potential in Examples 2 and 3

### Materials and Methods

### Cell Culture

MSC, cell product A and cell product B, cell product C fresh and cell products C cryo are obtained as described in Example 1.

### Flow Cytometry Analysis

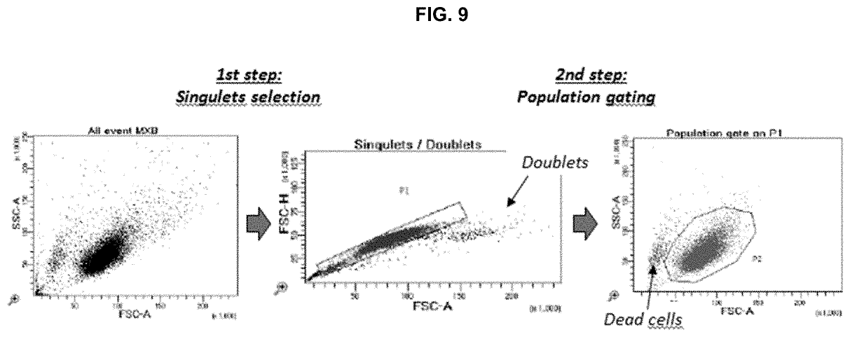

The characterization of cell surface markers was performed by flow cytometry. 50,000 cells at a concentration of 1×10⁶ cells/ml in PBS—1% BSA were incubated 10 min in the dark with 5 μl of antibodies. After this incubation time, cells were washed once with PBS. The different antibodies used for extracellular staining are the following: allophycocyanin (APC)-conjugated antibodies against CD105 (BD Biosciences®, Cat No: 562408), CD73 (BD Biosciences®, Cat No: 560847), Phycoerythrin (PE)-conjugated antibodies against CD10 (BD Biosciences®, Cat No: 555375), CD44 (BD Biosciences®, Cat No: 550989). Nonspecific staining was determined by incubating cells with immunoglobulin G (IgG) control conjugated with FITC, APC and PE (all BD Biosciences®, Cat No: 556649; 555751; 556650 respectively). Before analysis, gating of singulets and population of interest were performed as described in FIG. 9. The flow cytometry analysis was done on 10 000 events of the gated population using FACSCanto™II (BD Biosciences®) and FACSDiva™ 8.0 software (BD Biosciences®). Settings parameters used for the analysis were performed automatically with beads (BD CompBeads Plus®, Cat No 560497). For each conjugate, the positivity cut-off was fixed at 1% of positivity of the control isotype antibody and the positivity of each marker was determined. The median of fluorescence (MFI) of the whole analysed population was also determined and divided by the MFI of the corresponding isotype control antibody to obtain the normalized MFI (nMFI).

### Results

Flow cytometry analysis revealed that the general cell identity based on the cell surface marker expression profiles of cell product A, cell product B (generated with comparative methods according to the prior art), and cell products C with or without final cryopreservation (generated with a method illustrating the invention) were comparable.

All of them expressed the mesenchymal markers CD73, CD90 and CD105 and did not express the hematopoietic markers CD45, CD34 and CD3 (less than 5% of the cell population expressed these markers) (Table 4). Cell product A, cell product B and cell products C (with or without final cryopreservation) expressed low levels of MHC class II cell surface receptor such as the HLA-DR. Weak immunogenicity represented by weak expression of HLA-DR advantageously allows cell transplantation for instance to allogeneic subjects (Table 4). In addition, cell product A, cell product B, and cell products C (with or without final cryopreservation) highly expressed the enzyme ALP on their surface compared to undifferentiated MSCs (Tables 4 and 5). The high expression of ALP highlights the commitment of cell product A, cell product B, and cell products C (with or without final cryopreservation) toward the osteoblastic lineage. Cell product A, cell product B, and cell products C (with or without final cryopreservation) did also highly express the cell marker CD10 compared to undifferentiated MSCs (Table 4).

TABLE 4
Cell surface marker expression profile of comparative cell products (i.e. MSC, cell product
A, cell product B) and cell products illustrating the invention (i.e. cell products C)

| | | | | | | Cell product C cryopreserved | | | |
|---|---|---|---|---|---|---|---|---|---|
| Marker | | | Cell | Cell | Cell | | CS10 | | HTS |
| expression | | | product | product | product | | diluted | CS10 | 10% DMSO |
| (in %) | Statistics | MSCs | A | B | C fresh | CS10 | HSA 1:1 | 5% HSA | 10% HSA |
| CD73-APC | Mean | 100.0 | 100.0 | 100.0 | 100.0 | 100.0 | 100.0 | 100.0 | 100.0 |
| | Std Dev | 0.0 | 0.0 | 0.0 | 0.1 | 0.0 | 0.0 | 0.0 | 0.0 |
| | N | 6 | 11 | 22 | 15 | 10 | 2 | 3 | 3 |
| CD90-PE | Mean | 100.0 | 99.9 | 99.9 | 99.7 | 99.9 | 99.5 | 99.9 | 99.8 |
| | Std Dev | 0.1 | 0.2 | 0.2 | 0.6 | 0.2 | 0.7 | 0.2 | 0.4 |
| | N | 8 | 12 | 22 | 18 | 10 | 2 | 3 | 3 |
| CD105-APC | Mean | 100.0 | 99.8 | 100.0 | 99.9 | 100.0 | 100.0 | 100.0 | 100.0 |
| | Std Dev | 0.0 | 0.5 | 0.1 | 0.3 | 0.0 | 0.0 | 0.0 | 0.0 |
| | N | 8 | 12 | 20 | 18 | 10 | 2 | 3 | 3 |
| CD45-FITC | Mean | ND | ND | ND | 1.3 | 1.0 | 0.9 | 0.6 | 1.0 |
| | Std Dev | | | | 0.7 | 0.3 | 0.0 | 0.3 | 0.1 |
| | N | 0 | 0 | 0 | 16 | 10 | 2 | 3 | 3 |
| CD45-APC | Mean | 0.4 | 0.3 | 1.0 | ND | ND | ND | ND | ND |
| | Std Dev | 0.2 | 0.2 | 2.9 | | | | | |
| | N | 8 | 12 | 19 | 0 | 0 | 0 | 0 | 0 |
| CD34-APC | Mean | 0.6 | 1.0 | 1.6 | 2.1 | 1.6 | 2.8 | 3.3 | 1.5 |
| | Std Dev | 0.4 | 0.6 | 1.8 | 1.6 | 0.9 | 2.2 | 2.2 | 1.0 |
| | N | 8 | 12 | 22 | 16 | 10 | 2 | 3 | 3 |
| CD3-PE | Mean | 0.2 | 0.1 | 0.2 | 0.0 | 0.1 | 0.1 | 0.0 | 0.0 |
| | Std Dev | 0.1 | 0.1 | 0.1 | 0.1 | 0.1 | | 0.1 | 0.1 |
| | N | 6 | 10 | 17 | 16 | 10 | 1 | 3 | 3 |
| HLA-DR-PE | Mean | 0.7 | 1.0 | 1.8 | 1.6 | 0.9 | 1.4 | 1.4 | 1.4 |
| | Std Dev | 1.2 | 0.6 | 2.0 | 1.8 | 0.7 | 0.8 | 0.8 | 0.9 |
| | N | 8 | 12 | 22 | 16 | 10 | 2 | 3 | 3 |
| HLA-DR/DP/DQ-FITC | Mean | 1.0 | 1.6 | 1.6 | 2.0 | 1.5 | 1.8 | 1.8 | 1.6 |
| | Std Dev | 0.4 | 1.1 | 1.1 | 1.6 | 0.7 | 0.6 | 0.8 | 0.2 |
| | N | 8 | 12 | 22 | 16 | 10 | 2 | 3 | 3 |
| ALP-PE | Mean | 40.7 | 88.7 | 94.8 | 96.2 | 96.2 | 97.7 | 98.7 | 98.4 |
| | Std Dev | | 5.6 | 6.6 | 4.4 | 3.6 | 2.0 | 0.9 | 1.7 |
| | N | 1 | 5 | 10 | 18 | 10 | 3 | 2 | 3 |
| CD49e-PE | Mean | 92.7 | 99.6 | 99.8 | 99.9 | 100.0 | 99.9 | 99.8 | 99.9 |
| | Std Dev | 20.5 | 1.1 | 0.5 | 0.4 | 0.1 | 0.2 | 0.4 | 0.2 |
| | N | 8 | 12 | 19 | 18 | 10 | 3 | 2 | 3 |
| CD44-PE | Mean | 99.9 | 99.7 | 100.0 | 100.0 | 100.0 | 100.0 | 99.9 | 100.0 |
| | Std Dev | 0.2 | 0.5 | 0.0 | 0.1 | 0.0 | 0.1 | 0.2 | 0.1 |
| | N | 8 | 12 | 22 | 18 | 10 | 3 | 2 | 3 |
| CD10-PE | Mean | 19.6 | 99.6 | 98.8 | 99.3 | 99.5 | 99.3 | 99.1 | 98.9 |
| | Std Dev | 14 | 0.4 | 1.5 | 16 | 0.5 | 0.6 | 0.4 | 0.7 |
| | N | 10 | 12 | 25 | 16 | 10 | 2 | 3 | 3 |

Abbreviations:
ALP: alkaline phosphatase;
APC: allophycocyanin;
FITC: fluorescein isothiocyanate;
HLA-DR: human leukocyte antigen - DR isotype;
HLA-DR/DP/DQ: human leukocyte antigen -DR/DP/DQ isotypes;
MSC: mesenchymal stem cells;
ND: not determined;
PE: phycoerythrin;
SD: standard deviation

TABLE 5
ALP expression levels of comparative cell products (i.e. MSC, cell product A, cell
product B) and cell products illustrating the invention (i.e. cell products C)

| | | | | | | Cell product C cryopreservation | | | |
|---|---|---|---|---|---|---|---|---|---|
| | | | Cell | Cell | Cell | | CS10 | | HTS |
| | | | product | product | product | | diluted | CS10 | 10% DMSO |
| | Statistics | MSCs | A | B | C fresh | CS10 | HSA 1:1 | 5% HSA | 10% HSA |
| ALP-PE | Mean | 40.7 | 88.7 | 94.8 | 96.2 | 96.2 | 98.7 | 97.7 | 98.4 |
| population | Std Dev | | 5.6 | 6.6 | 4.4 | 3.6 | 0.9 | 2.0 | 1.7 |
| positivity (%) | N | 1 | 5 | 10 | 18 | 10 | 2 | 3 | 3 |
| ALP-PE cell | Mean | 2.4 | 19.8 | 56.1 | 60.3 | 38.0 | 57.7 | 42.7 | 41.2 |
| surface | Std Dev | | 10.8 | 27.4 | 38.9 | 24.2 | 43.1 | 37.2 | 31.3 |
| expression | N | 1 | 5 | 10 | 18 | 10 | 2 | 3 | 3 |
| level (nMFI) | | | | | | | | | |
| ALP enzymatic | Mean | 176.3 | 671.9 | 874.7 | 895.5 | 801.5 | ND | 719.9 | 633.6 |
| activity | Std Dev | 252.9 | 305.8 | 772.9 | 387.3 | 351.3 | | 277.0 | 263.9 |
| (mU/mg of | N | 3 | 9 | 26 | 12 | 5 | 0 | 2 | 2 |
| total protein) | | | | | | | | | |

Abbreviations:
ALP: alkaline phosphatase;
ND: not determined;
PE: phycoerythrin;

The cell surface marker expression profile was not only characterized by the presence of markers on cell surface (population positivity percentage) but also by analysing the quantity of markers expressed on cell surface (population normalized median of fluorescence) of different markers. These analyses highlighted some differences between the different MSC-derived cells.

Cell product B and cell products C (with or without final cryopreservation) cultured in presence of heparin expressed higher level of ALP than MSCs and cell product A cultured in absence of heparin (ALP-PE nMFI results) strengthening their commitment toward the osteoblastic lineage of bone-forming cells.

The expression of the mesenchymal markers CD73 and CD105 on cell surface were also dependent on the cell types. Cell products generated in presence of heparin (cell product B and cell products C with or without final cryopreservation) expressed higher level of CD73 and CD105 than cell product A. In addition, cell products C seemed to possess more CD73 and CD105 on their surface than cell product B especially when cell product C did not undergo a final cryopreservation (Table 6). Differentiated cell products A, B and C express higher amount of cell marker CD10 than undifferentiated MSC. In addition, cell products C possess more CD10 on their surface than cells products A and B (Table 6).

In view of the above, it appears that cell product B and cell products C (with or without final cryopreservation), of which product B and C has shown an osteoinductive and a high osteogenic potential in Examples 2 and 3, can be distinguished from cell product A, of which product A has shown an osteoinductive and a low osteogenic potential in Examples 2 and 3, based on the quantity expressing any one or more of CD73, CD105, CD10 or CD44; and/or the quantity of any one or more of CD73, CD105 or CD44 expressed on the cell surface.

More particularly, based on Tables 4 and 6 it appears that at least 90% of the cells of cell product B and cell products C express CD73, CD105, CD10 and CD44 on the cell surface (Table 4) and that the cells of cell product B and cell products C have a nMFI_{CD73} of at least 500, a nMFI_{CD44} of at least 100 (or at least 150), and a nMFI_{CD105} of at most 150 (Table 6). On the other hand, cells of cell product A have a nMFI_{CD73} of less than 500 and a nMFI_{CD44} of less than 100 (or less than 150); and MSCs have a nMFI_{CD73} of less than 500 and a nMFI_{CD105} of more than 150 (Table 6).

Accordingly, the quantity of in vitro differentiated cells expressing CD73, CD105, CD10 and CD44 in combination with the quantity of CD73, CD105 and CD44 expressed by in vitro differentiated cells is suitable and sufficient for distinguishing cells of cell product B and cell products C from MSCs or cells of cell product A. In line therewith, the quantity of in vitro differentiated cells expressing CD73, CD105, CD10 and CD44 in combination with the quantity of CD73, CD105 and CD44 expressed by in vitro differentiated cells is suitable and sufficient for determining whether in vitro differentiated cells have osteogenic potential, and especially for determining whether in vitro differentiated cells have a high osteogenic potential.

TABLE 6
Additional cell surface marker expression results of comparative cell
products (i.e. MSC, cell product A, cell product B) and cell products C

| | | | | | | Cell product C cryopreservation | | | |
|---|---|---|---|---|---|---|---|---|---|
| Marker | | | Cell | Cell | Cell | | CS10 | | HTS |
| expression | | | product | product | product | | diluted | CS10 | 10% DMSO |
| (in nMFI) | Statistics | MSCs | A | B | C fresh | CS10 | HSA 1:1 | 5% HSA | 10% HSA |
| ALP-PE | Mean | 2.4 | 19.8 | 56.1 | 60.3 | 38.0 | 57.7 | 42.7 | 41.2 |
| | Std Dev | / | 10.8 | 27.4 | 38.9 | 24.2 | 43.1 | 37.2 | 31.3 |
| | N | 1 | 5 | 10 | 18 | 10 | 2 | 3 | 3 |
| CD73-APC | Mean | 234.8 | 130.7 | 646.3 | 996.9 | 703.9 | 777.8 | 719.2 | 784.2 |
| | Std Dev | 84.3 | 80.1 | 138.8 | 181.1 | 150.0 | 73.3 | 90.6 | 47.3 |
| | N | 6 | 11 | 22 | 15 | 10 | 2 | 3 | 3 |
| | Mean | 207.7 | 26.6 | 59.1 | 99.7 | 70.2 | 74.6 | 72.6 | 67.0 |
| CD105-APC | Std Dev | 67.6 | 15.2 | 13.1 | 27.0 | 13.1 | 2.3 | 4.2 | 3.9 |
| | N | 8 | 12 | 20 | 18 | 10 | 2 | 3 | 3 |
| CD44-PE | Mean | 139.8 | 62.0 | 156.6 | 362.8 | 378.4 | 185.5 | 227.5 | 261.3 |
| | Std Dev | 57.5 | 19.1 | 40.7 | 250.4 | 205.3 | 19.6 | 80.1 | 147.7 |
| | N | 8 | 12 | 22 | 18 | 10 | 2 | 3 | 3 |
| CD49e-PE | Mean | 81.0 | 22.5 | 33.5 | 44.3 | 39.6 | 35.7 | 35.2 | 36.87 |
| | Std Dev | 51.4 | 9.9 | 11.0 | 8.0 | 7.4 | 3.3 | 3.5 | 10.0 |
| | N | 8 | 12 | 19 | 18 | 10 | 2 | 3 | 3 |
| HLA-ABC-FITC | Mean | 26.1 | 21.6 | 80.2 | 115.7 | 104.7 | 71.5 | 79.5 | 70.1 |
| | Std Dev | / | 5.2 | 17.4 | 22.0 | 24.9 | 27.2 | 22.1 | 18.0 |
| | N | 1 | 4 | 8 | 16 | 10 | 2 | 3 | 3 |
| CD10-PE | Mean | 0.8 | 36.2 | 32.2 | 64.0 | 59.3 | 63.8 | 64.1 | 57.9 |
| | Std Dev | 1.1 | 16.4 | 16.8 | 38.5 | 30.5 | 45.5 | 35.4 | 32.9 |
| | N | 8 | 12 | 22 | 18 | 10 | 2 | 3 | 3 |

Abbreviations:
ALP: alkaline phosphatase;
APC: Allophycocyanin;
FITC: Fluorescein isothiocyanate;
HLA-ABC: Human Leukocyte Antigen ABC;
HLA-DR: Human Leukocyte Antigen - DR isotype;
MSC: mesenchymal stem cells;
NA: not available;
ND: not determined;
PE: phycoerythrin;
SD: standard deviation

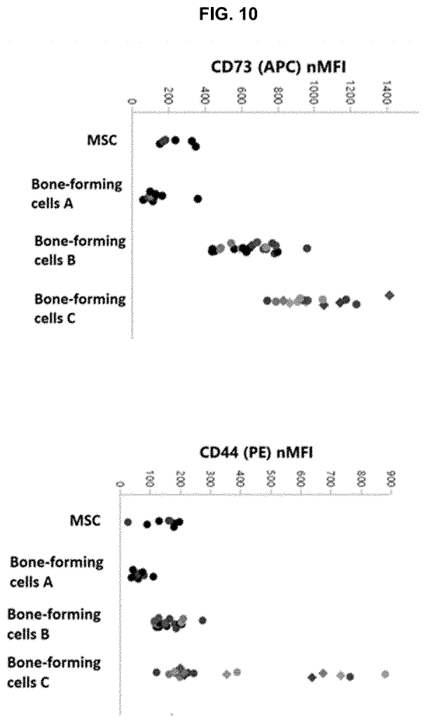

Furthermore, the nMFI flow cytometry analysis revealed that CD73 and CD44 protein expressions were more elevated in bone-forming cells C than in the other cell types (FIG. 10), including bone-forming cells B.

1. A method of detecting in vitro differentiated cells having osteogenic potential, the method comprising:
detecting a normalized Median of Fluorescence Intensity (nMFI) for CD10 of at least 40 on the cell surface of the in vitro differentiated cells.

2. The method according to claim 1, wherein the method comprises detecting a nMFI for CD10 of at least 50.

3. The method according to claim 1, the method further comprising:
and
detecting that at least 90% of the in vitro differentiated cells express, on the cell surface, one or more of CD73, CD105, CD10 or CD44.

4. The method according to claim 1, the method comprising:
detecting that at least 90% of the in vitro differentiated cells express, on the cell surface, CD73, CD105, CD10 and CD44.

5. The method according to claim 1, wherein the method further comprises measuring the quantity of any one or more of CD73, CD105 or CD44 expressed by the in vitro differentiated cells.

6. The method according to claim 5, wherein the method comprises detecting that the in vitro differentiated cells have one or more of
a nMFI for CD73 of at least 500,
a nMFI for CD44 of at least 100, or
a nMFI for CD105 of at most 150.

7. The method according to claim 1, wherein the method further comprises measuring the quantity of CD73, CD105 and CD44 expressed by the in vitro differentiated cells.

8. The method according to claim 6, wherein the method comprises determining that the in vitro differentiated cells have osteogenic potential if detecting that the in vitro differentiated cells have
a nMFI for CD73 of at least 500,
a nMFI for CD44 of at least 100, and
a nMFI for CD105 of at most 150.

9. (canceled)

10. (canceled)

11. The method according to claim 1, wherein the in vitro differentiated cells are differentiated from mesenchymal stem cells (MSC).

12. The method according to claim 1, wherein the in vitro differentiated cells are human cells.

13. A method of preparing in vitro differentiated cells of chrondro-osteoblastic lineage, the method comprising:
recovering mesenchymal stem cells (MSC) from a biological sample of a subject;
obtaining in vitro differentiated cells from the MSC;
determining that the in vitro differentiated cells have osteogenic potential by the method according to claim 1; and
preparing, from cells isolated from the subject, cells of the chondro-osteoblastic lineage.

14. The method according to claim 13, wherein the subject is a human subject.

15. The method according to claim 1, wherein the nMFI_{CD10} is measured with an excitation wavelength of 488 nm and an emission wavelength of 580 nm for phycoerythrin.

16. The method according to claim 2, wherein the nMFI_{CD10} is measured with an excitation wavelength of 488 nm and an emission wavelength of 580 nm for phycoerythrin.

17. The method according to claim 3, wherein the nMFI_{CD10} is measured with an excitation wavelength of 488 nm and an emission wavelength of 580 nm for phycoerythrin.

18. The method according to claim 4, wherein the nMFI_{CD10} is measured with an excitation wavelength of 488 nm and an emission wavelength of 580 nm for phycoerythrin.

19. The method according to claim 6,
wherein the nMFI_{CD10} is measured with an excitation wavelength of 488 nm and an emission wavelength of 580 nm for phycoerythrin, the nMFI_{CD73} is measured with an excitation wavelength of 633 nm and an emission wavelength of 660 nm for allophycocyanin, the nMFI_{CD44} is measured with an excitation wavelength of 488 nm and an emission wavelength of 580 nm for phycoerythrin, the nMFI₁₀₅ is measured with an excitation wavelength of 633 nm and an emission wavelength of 660 nm for allophycocyanin.

20. The method according to claim 8,
wherein the nMFI_{CD10} is measured with an excitation wavelength of 488 nm and an emission wavelength of 580 nm for phycoerythrin, the nMFI_{CD73} is measured with an excitation wavelength of 633 nm and an emission wavelength of 660 nm for allophycocyanin, the nMFI_{CD44} is measured with an excitation wavelength of 488 nm and an emission wavelength of 580 nm for phycoerythrin, the nMFI₁₀₅ is measured with an excitation wavelength of 633 nm and an emission wavelength of 660 nm for allophycocyanin.
